(19) United States
(12) Patent Application Publication
(10) Pub. No.: US 2003/0050268 A1
(43) Pub. Date: Mar. 13, 2003

(54) Immunostimulatory nucleic acid for treatment of non-allergic inflammatory diseases

(76) Inventors: Arthur M. Krieg, Wellesley, MA (US); Daniel J. Berg, Iowa City, IA (US)

Correspondence Address: WOLF GREENFIELD & SACKS, PC, FEDERAL RESERVE PLAZA, 600 ATLANTIC AVENUE, BOSTON, MA, 02210-2211, US

(21) Appl. No.: 10/112,653

(22) Filed: Mar. 29, 2002

Related U.S. Application Data
(60) Provisional application No. 60/279,642, filed on Mar. 29, 2001.

(51) Int. Cl. A61K 48/00

(52) U.S. Cl. 514/44

(57) ABSTRACT

The invention provides methods and compositions for using immunostimulatory nucleic acids to treat non-allergic inflammatory diseases. Non-allergic inflammatory diseases that may be treated according to the methods and products of the invention include psoriasis and inflammatory bowel disease. The invention further provides methods for augmenting a Th1 response to immunostimulatory nucleic acid involving inhibition of prostaglandin-mediated counter-regulatory response.

### RELATED APPLICATION

This invention claims benefit of United States Provisional Patent Application Serial No. 60/279,642, filed Mar. 29, 2001, the entire contents of which are incorporated herein by reference.

### FIELD OF THE INVENTION

The present invention relates to immunostimulatory nucleic acids and methods of using the immunostimulatory nucleic acids in the treatment of non-allergic inflammation.

### BACKGROUND OF THE INVENTION

Inflammation is an infiltrative type of immune cell-mediated host defense mechanism that, unlike acquired immunity, lacks both antigen specificity and antigen memory. In many respects inflammation is a type of natural or innate immunity, mediated by a combination of certain types of immune cells and secreted products of immune cells. The immune cells principally involved in inflammation include granulocytes (neutrophils, eosinosphils, and basophils), phagocytic cells (monocytes and macrophages), natural killer (NK) cells, and T lymphocytes (T cells). Monocytes and macrophages phagocytose materials foreign to the host and degrade them within lysosomes. These cells also secrete enzymes, reactive oxygen species, and lipid mediators including leukotrienes and prostaglandins, all of which can not only serve to protect the host but also can cause unwanted damage to uninvolved bystander cells. The inflammatory response further includes the recruitment and localization of neutrophils and other inflammatory cells, under the direction of cytokines and chemokines secreted by the monocytes and macrophages. Among the cytokines involved in promoting inflammation are interferon (IFN)-α, IFN-β, IFN-γ, tumor necrosis factor (TNF)-α, TNF-β, interleukin (IL)-1β, IL-6, IL-8, and IL-12. Conversely, anti-inflammatory cytokines are believed to include IL-10. Additional soluble factors released as part of the inflammatory response include certain plasma proteases, including complement; vasoactive kinins, including bradykinin; and clotting and fibrinolytic factors (factor XII and plasmin).

Allergy represents a special subtype of inflammation, usually characterized by a central role of immunoglobulin E (IgE). The immune cells principally involved in allergy include mast cells, basophils, eosinophils, and monocytes. Allergic responses are sometimes viewed as having separate phases, including acute allergic reaction, late-phase allergic reaction, and chronic allergic inflammation. Chronic allergic inflammation is more like non-allergic inflammation than the acute and late-phase allergic response. Examples of IgE-associated allergic diseases in humans include anaphylaxis, allergic rhinitis (hayfever), allergic asthma, and atopic dermatitis.

Thus inflammation may be divided broadly into allergic and non-allergic inflammation, whereby a central role of IgE is implicated in at least the acute phase of allergic inflammation. Examples of non-allergic inflammation include psoriasis, inflammatory bowel disease (IBD, including Crohn's disease and ulcerative colitis), and many types of autoimmune disease.

Two T helper (Th) lymphocyte subsets, Th1 and Th2, are defined by the cytokines they elaborate upon stimulation. Th1 and Th2 cytokines are generally viewed as reciprocally, counter-regulatory cytokines, produced by lymphocytes as well as other immune and non-immune cells, which skew a developing immune response toward either a cell-mediated Th1-type response or a humoral Th2-type response. Th1 cytokines include IFN-γ, IL-2, IL-12, IL-18, and TNF; Th2 cytokines include IL-4, IL-5, IL-10, and IL-13. Thus while many cytokines are classified neither as Th1 nor Th2, T lymphocytes can participate in the initiation and regulation of inflammation through their elaboration of cytokines, including IL-2, IL-4, IL-6, IFN-γ, TNF-α, and transforming growth factor (TGF)-β.

In addition to its role in promoting inflammation, IL-12 is a key Th1 cytokine secreted by macrophages and antigen-presenting cells (APCs). IL-12 enhances secretion of IFN-γ and proliferation of NK cells, Th1 cells, and cytotoxic T lymphocytes (CTLs). Trinchieri G (1998) Int Rev Immunol 16:365-96. IL-12 signaling occurs through a β₁/β₂ heterodimeric IL-12 receptor (IL-12R) which, when cross-linked, leads to activation of the kinases Jak2 and Tyk2, as well as to activation and nuclear translocation of signal transduction and activator of transcription (Stat)3 and Stat4, resulting in induction of IFN-γ and TNF.

In contrast to the proinflammatory Th1 cytokines, the Th2 cytokines IL-10 and IL-4 are believed to be anti-inflammatory cytokines inasmuch as they downregulate macrophage activation and inhibit IL-6 production.

Diseases involving non-allergic inflammation such as psoriasis, Crohn's disease, and ulcerative colitis are also believed to be caused in part by Th I-mediated immune responses. Consistent with the observation that Th1 and Th2 cytokines are reciprocally counter-regulatory, Th1 cytokines or agents that induce Th1-like immune activation are effective in treating or preventing Th2-mediated allergic inflammatory diseases such as asthma. It would be surprising, by the same reasoning, that Th1 cytokines or agents that induce Th1-like immune activation should be effective in treating or preventing Th1-mediated non-allergic inflammatory diseases.

### SUMMARY OF THE INVENTION

In one aspect the invention provides a method for treating a non-allergic inflammatory disease. The method involves administering to a subject having or at risk of developing a non-allergic inflammatory disease a therapeutically effective amount of an immunostimulatory nucleic acid to treat or prevent the non-allergic inflammatory disease. In a preferred embodiment the therapeutically effective amount of an immunostimulatory nucleic acid reduces or prevents non-allergic inflammation in a tissue of the subject. The non-allergic inflammation is preferably independent of IgE crosslinking, as discussed further below.

Certain types of tissues may be involved in non-allergic inflammatory disease. According to this and other aspects of the invention, in some embodiments the non-allergic inflammatory disease involves an epithelium. In some embodiments the epithelium involved in the non-allergic inflammatory disease is a mucosal epithelium.

Certain specific diseases can be treated according to this and other aspects of the invention. In certain preferred embodiments of this and other aspects of the invention, the non-allergic inflammatory disease can include psoriasis, eczema, allergic contact dermatitis, latex dermatitis, and inflammatory bowel disease. As explained further below, allergic contact dermatitis, because it is characterized by an inflammatory response essentially independent of an IgE response, is a non-allergic inflammatory disease. In a preferred embodiment the non-allergic inflammatory disease is psoriasis. In a preferred embodiment the non-allergic inflammatory disease is allergic contact dermatitis. In a preferred embodiment the non-allergic inflammatory disease is latex dermatitis.

Certain types of immunostimulatory nucleic acid are useful according to this and other aspects of the invention. For example, in certain embodiments the immunostimulatory nucleic acid is a CpG nucleic acid, as will be defined further below. In certain embodiments the immunostimulatory nucleic acid is a methylated CpG nucleic acid. In certain embodiments the immunostimulatory nucleic acid is a T-rich nucleic acid. In certain embodiments the immunostimulatory nucleic acid is a poly-G nucleic acid. In certain embodiments in which the immunostimulatory nucleic acid is a poly-G nucleic acid, the poly-G nucleic acid includes the formula 5′-X₁X₂GGGX₃X₄-3′ wherein each of X₁, X₂, X₃, and X₄ is any nucleotide other than G. In certain embodiments such poly-G nucleic acids preferably do not include any of the formulas 5′-GXGGG-3′,5′-XGGGG-3′, or 5′-GGGXG-3′, wherein X is any nucleotide. These types of immunostimulatory nucleic acids are also defined further below.

In preferred embodiments the immunostimulatory nucleic acid of this and other aspects of the invention is a synthetic nucleic acid.

Also according to preferred embodiments of this and other aspects of the invention, the immunostimulatory nucleic acid comprises at least one stabilized internucleotide linkage. Thus in certain preferred embodiments the at least one stabilized internucleotide linkage is a phosphorothioate linkage. In certain preferred embodiments the at least one stabilized internucleotide linkage is a phosphorodithioate linkage. In certain preferred embodiments the at least one stabilized internucleotide linkage is a p-ethoxy linkage. The immunostimulatory nucleic acid can in some embodiments have a backbone completely made up of stabilized internucleotide linkages, e.g., a completely phosphorothioate backbone.

Preferably, the T-rich and poly-G nucleic acids are also CpG nucleic acids. In certain embodiments the immunostimulatory nucleic acid comprises a poly-G motif (e.g., 5′-GGGG-3′) and a palindrome. Preferably, the immunostimulatory nucleic acid comprises two poly-G motifs, one 5′ and one 3′ to a centrally located palindrome sequence, with a chimeric backbone (i.e., a backbone that is partially, but not completely, composed of phosphorothioate linkages). In some embodiments, a plurality of immunostimulatory nucleic acids is administered, wherein the plurality comprises a CpG nucleic acid and a T-rich nucleic acid, or a CpG nucleic acid and a poly-G nucleic acid, a T-rich nucleic acid and a poly-G nucleic acid, a combination of different CpG nucleic acids, a combination of different poly-G nucleic acids, or a combination of different T-rich nucleic acids.

The immunostimulatory nucleic acid may be between 6 and 100 nucleotides long. In certain preferred embodiments, the immunostimulatory nucleic acid comprises between 8 and 40 nucleotides.

According to this and other aspects of the invention, in some embodiments the immunostimulatory nucleic acid induces IL-12.

According to this and other aspects of the invention, in some embodiments the immunostimulatory nucleic acid induces IFN-α.

According to this and other aspects of the invention, in some embodiments the immunostimulatory nucleic acid induces IFN-γ.

According to this and other aspects of the invention, in some embodiments the immunostimulatory nucleic acid induces IL-10.

In certain embodiments the method can employ a plurality of immunostimulatory nucleic acids, including a plurality of nucleic acids of a single type, e.g., CpG immunostimulatory nucleic acids, and a plurality of nucleic acids of different types, e.g., CpG and T-rich immunostimulatory nucleic acids. In certain embodiments, a plurality of different types of immunostimulatory nucleic acid motifs can be present either in a single oligonucleotide or in different oligonucleotides.

Different modes and routes of administration are contemplated. In some embodiments the immunostimulatory nucleic acid is administered locally to damaged epithelium. Alternatively in some embodiments the immunostimulatory nucleic acid is administered locally to intact epithelium. In certain embodiments the immunostimulatory nucleic acid is administered systemically. Preferred routes of administration include oral, parenteral, topical, subcutaneous, and transdermal administration.

The immunostimulatory nucleic acid may be administered to the subject in conjunction with administering to the subject an anti-inflammatory agent selected from the group consisting of: anti-inflammatory corticosteroids, nonsteroidal anti-inflammatory drugs (NSAIDs), salicylates, cyclooxygenase inhibitors (coxibs), vitamin A analogs, vitamin D analogs, retinoids, cytokines, agonists of cytokines, antagonists of cytokines, agonists of cytokine receptors, antagonists of cytokine receptors, cytokine receptor analogs, antibodies specific for cytokines, antibodies specific for cytokine receptors, and immunosuppressive agents, such as cyclosporine A, FK506, and methotrexate.

In another aspect the invention provides a method for treating inflammatory bowel disease. The method according to this and other aspects involves administering to a subject having or at risk of developing an inflammatory bowel disease a therapeutically effective amount of an immunostimulatory nucleic acid to treat or prevent the inflammatory bowel disease. In one embodiment of this and other aspects of the invention, the inflammatory bowel disease is ulcerative colitis. In one embodiment the inflammatory bowel disease is Crohn's disease.

In some embodiments the immunostimulatory nucleic acid is administered locally to damaged mucosal epithelium. Alternatively in some embodiments the immunostimulatory nucleic acid is administered locally to intact mucosal epithelium. In certain embodiments the immunostimulatory nucleic acid is administered systemically. Preferred routes of administration include oral, rectal, topical, and parenteral administration.

In preferred embodiments the immunostimulatory nucleic acid can be administered to the subject in conjunction with administering to the subject an anti-inflammatory agent selected from the group consisting of 5-aminosalicylate, agents containing 5-aminosalicylate, anti-inflammatory corticosteroids, NSAIDs, coxibs, cytokines, agonists of cytokines, antagonists of cytokines, agonists of cytokine receptors, antagonists of cytokine receptors, cytokine receptor analogs, antibodies specific for cytokines, antibodies specific for cytokine receptors, and immunosuppressive agents, such as cyclosporine A, FK506, and methotrexate.

A pharmaceutical composition is provided according to another aspect of the invention. The pharmaceutical composition includes an immunostimulatory nucleic acid in an effective amount for preventing or treating an immune or inflammatory response associated with a non-allergic inflammatory disease, a non-allergic inflammatory disease medicament, and a pharmaceutically acceptable carrier.

In certain embodiments the non-allergic inflammatory disease is selected from the group consisting of: psoriasis, eczema, allergic contact dermatitis, and inflammatory bowel disease. In a preferred embodiment the non-allergic inflammatory disease is psoriasis. In a preferred embodiment the non-allergic inflammatory disease is allergic contact dermatitis. In certain embodiments the non-allergic inflammatory disease is latex dermatitis.

The non-allergic inflammatory disease medicament may be any of the following: 5-aminosalicylate, agents containing 5-aminosalicylate, anti-inflammatory corticosteroids, NSAIDs, coxibs, vitamin A analogs, vitamin D analogs, retinoids, cytokines, agonists of cytokines, antagonists of cytokines, agonists of cytokine receptors, antagonists of cytokine receptors, cytokine receptor analogs, antibodies specific for cytokines, antibodies specific for cytokine receptors, and immunosuppressive agents, such as cyclosporine A, FK506, and methotrexate.

In another aspect a pharmaceutical composition suitable for topical administration is provided. The pharmaceutical composition according to this and other aspects of the invention includes an immunostimulatory nucleic acid in an effective amount for preventing or treating an immune response associated with a non-allergic inflammatory disease and a pharmaceutically acceptable carrier, formulated as a lotion, cream, ointment, gel, or transdermal patch.

In certain embodiments the non-allergic inflammatory disease is selected from the group consisting of: psoriasis, eczema, allergic contact dermatitis, and latex dermatitis. In a preferred embodiment the non-allergic inflammatory disease is psoriasis. In a preferred embodiment the non-allergic inflammatory disease is allergic contact dermatitis. In a preferred embodiment the non-allergic inflammatory disease is latex dermatitis.

In another aspect the invention provides a method of augmenting Th1-like immune activation induced by an immunostimulatory nucleic acid. The method according to this and other aspects involves contacting an immune cell with an effective amount of an immunostimulatory nucleic acid to induce Th1-like immune activation, and contacting the immune cell with an inhibitor of cyclooxygenase-2 (COX-2) expression, in an amount effective to augment Th1-like immune activation induced by the immunostimulatory nucleic acid.

In one embodiment the immunostimulatory nucleic acid is administered to a subject in need of Th1-like immune activation in an effective amount to induce Th1-like immune activation, and the inhibitor of COX-2 expression is administered to the subject in an effective amount to augment Th1-like immune activation induced by the immunostimulatory nucleic acid.

According to yet another aspect of the invention, a method of augmenting Th1-like immune activation induced by an immunostimulatory nucleic acid is provided. The method according to this and other aspects involves contacting an immune cell with an effective amount of an immunostimulatory nucleic acid to induce Th1-like immune activation, and contacting the immune cell with an agent that inhibits prostaglandin E2 (PGE₂) signaling through its receptor, in an amount effective to augment the Th1-like immune activation induced by the immunostimulatory nucleic acid.

In one embodiment the agent that inhibits PGE₂ signaling through its receptor is an antibody that binds specifically to PGE₂.

In one embodiment the immunostimulatory nucleic acid is administered to a subject in need of Th1-like immune activation in an effective amount to induce Th1-like immune activation, and the agent that inhibits PGE₂ signaling through its receptor is administered to the subject in an effective amount to augment the Th1-like immune activation.

In yet another aspect, the invention provides a method of augmenting Th1-like immune activation in a subject. The method according to this and other aspects entails administering to a subject in need of Th1-like immune activation an effective amount of an immunostimulatory nucleic acid to induce Th1-like immune activation, and administering to the subject an effective amount of a cyclooxygenase inhibitor to inhibit prostaglandin expression, wherein the subject is free of symptoms of asthma or allergy otherwise calling for treatment with immunostimulatory nucleic acid, and wherein Th1-like immune activation induced by administering the immunostimulatory nucleic acid and the cyclooxygenase inhibitor is greater than Th1 -like immune activation induced by administering the immunostimulatory nucleic acid alone.

In a preferred embodiment the prostaglandin is PGE₂.

In some embodiments the cyclooxygenase inhibitor is a nonsteroidal anti-inflammatory drug. In some embodiments the cyclooxygenase inhibitor is a selective inhibitor of COX-2 catalytic activity.

### BRIEF DESCRIPTION OF THE FIGURES

FIG. 1 is a pair of bar graphs depicting PGE₂ production from oligodeoxynucleotide (ODN)-stimulated spleen cells (A) and RAW 264.7 macrophages (B), in the presence (+NS-398) or absence of cyclooxygenase 2 (COX-2) inhibitor. Control is media unsupplemented with ODN; ODN 1982 is a control ODN (SEQ ID NO:217); ODN 1826 is a CpG ODN (SEQ ID NO:63).

FIG. 2 is a trio of Western immunoblot gel images depicting the effect of ODN on COX-2 protein expression in spleen cells (A) and RAW 264.7 macrophage cells (B and C).

FIG. 3 is a graph depicting the effect of selective COX-2 inhibitor SC-58236 on CpG DNA-induced IFN-γ secretion in vivo.

### DETAILED DESCRIPTION OF THE INVENTION

The present invention provides methods useful for the treatment and prevention of non-allergic inflammatory diseases, including psoriasis, eczema, allergic contact dermatitis, latex dermatitis, and inflammatory bowel disease. These diseases are believed to be caused in part by Th1-mediated immune responses. The methods involve the administration of certain immunostimulatory nucleic acids to subjects that have or are at risk of developing a non-allergic inflammatory disease. Surprisingly, the isolated immunostimulatory nucleic acids useful in the methods for treating and preventing non-allergic inflammatory diseases are known to induce or redirect an immune response toward a Th1-like type of immune response. Since CpG DNA and T-rich oligodeoxynucleotide (ODN) cause Th1-mediated immune activation, it was unexpected that these should be effective in treating these diseases. However, in addition to the Th1 effects, CpG DNA also induces counterregulatory mechanisms, including the generation of IL-10. Redford T W et al. (1998) J Immunol 161:3930-5. Also surprisingly, the isolated immunostimulatory nucleic acids useful in the methods for treating and preventing non-allergic inflammatory diseases are useful when administered systemically to a subject in need of such treatment.

The present invention also provides methods useful for enhancing a Th1-like immune response to immunostimulatory nucleic acids. It has surprisingly been discovered according to aspects of the present invention that immunostimulatory nucleic acids that induce a Th1-like immune response also induce counter-regulatory molecules, prostaglandins, that may dampen the net Th1 response. The prostaglandins are induced by immunostimulatory nucleic acid-induced up-regulation of expression of prostaglandin synthase, a key enzyme in the synthetic pathway from arachidonic acid to prostaglandin. Thus, inhibition of this pathway and of prostaglandin signaling in the context of immunostimulatory nucleic acids removes the counter-regulatory effect of prostaglandins on Th1-like immune activation.

Allergy involves the clinically adverse reaction to allergens which reflects the expression of acquired immunologic responsiveness involving allergen-specific antibodies and/or T lymphocytes. Classically, allergy was divided into four types, including type I (immediate hypersensitivity) involving IgE antibodies, and type IV (delayed-type hypersensitivity, DTH) involving sensitized T lymphocytes without any essential role for antibodies. Coombs RRA and Gell PGH (1963) The classification of allergic reactions underlying disease. In: Gell PGH and Coombs RRA, eds. Clinical Aspects of Immunology. Oxford: Blackwell Scientific Publications, pp. 317-37. As used herein, an allergic inflammatory response will refer to IgE-associated immune response that includes the development of a prominent IgE antibody response to an initiating allergen, be the allergen known or unknown. Thus as used herein an allergic inflammatory response corresponds most closely to the classical type I allergic response and does not include the classical type W allergic response (DTH).

The term “non-allergic inflammatory disease,” as used herein, refers to a disease or disorder of a subject, wherein the disease or disorder is characterized by an inflammatory response essentially independent of an IgE response. By essentially independent of an IgE response is meant that local or systemic levels of IgE, if measured, would not be significantly different from corresponding local or systemic levels in subjects without an inflammatory response. Such non-allergic inflammatory diseases or disorders typically include an inflammatory response to an antigen, which may be known or unknown, that is characterized by infiltration and activation of certain immune cells, including neutrophils, monocytes, macrophages, NK cells, and T cells and by secreted products of those cells, including but not limited to IFN, TNF, IL-1, IL-6, IL-8, and IL-12. The inflammatory response of the non-allergic inflammatory disease may be acute or chronic and it may be intermittent or recurrent. Examples of non-allergic inflammatory diseases include, without limitation, psoriasis, inflammatory bowel disease, eczema, allergic contact dermatitis, latex dermatitis, and autoimmune disorders. In some instances the non-allergic inflammatory disease may encompass chronic allergic inflammation, insofar as this entity is essentially independent of an IgE response.

The non-allergic inflammatory disease in certain preferred embodiments involves an epithelium. An epithelium is a tissue composed of closely aggregated cells that are in apposition over a large part of their surface and so form a continuous layer of cells covering and defining an external or internal surface. An epithelium may include more than one layer and more than one kind of cell. Certain tissues and organs have specialized forms of epithelium, e.g., simple, stratified and pseudostratified; squamous, cuboidal, columnar, and transitional. Epithelia may also have associated specialized structures and functions, such as microvilli, cilia, and glands. The surface of the skin, or epidermis, is a stratified squamous epithelium. In addition, the epithelium lining the gastrointestinal tract is termed a mucosal epithelium, consisting of an epithelial lining (associated and in communication with mucosal and submucosal glands) overlying a loose connective tissue layer (lamina propria) and a thin layer of smooth muscle (muscularis mucosa). Thus in certain preferred embodiments the non-allergic inflammatory disease involves the skin, and in certain preferred embodiments the non-allergic inflammatory disease involves the mucosa lining the gastrointestinal tract.

Psoriasis is a chronic inflammatory disease of the skin that affects 1-3% of the Caucasian population worldwide. Barker J N W N (1994) Bailliere's Clin Rheumatol 8:429-37. This complex disease is characterized by alterations in a variety of different cells of the skin. These include epidermal keratinocyte hyperproliferation and altered differentiation indicated by focal parakeratosis (cell nuclei in stratum corneum), aberrant expression of the hyperproliferation-associated keratin pair 6/16 (Stoler A et al. (1988) J Cell Biol 107:427-46; Weiss R A et al. (1984) J Cell Biol 98:1397-1406), involucrin and filaggrin (Bernard B A et al. (1986) Br J Dermatol 114:279-83; Dover R et al. (1987) J Invest Dermatol 89:349-52; Ishida-Yamamoto A et al. (1995) J Invest Dermatol 104:391-5), and integrin adhesion molecules (VLA-3, -5 and -6, α₆β₄) (Hertle M D et al. (1992) J Clin Invest 89:1892-1901; Kellner I et al. (1991) Br J Dermatol 125:211-6). In addition, de novo expression of major histocompatibility complex (MHC) class II and intercellular adhesion molecule-1 (ICAM-1, CD54) by keratinocytes is observed (Barker J N W N et al. (1990) J Clin Invest 85:605-8; Gottlieb A B et al. (1986) J Exp Med 164:1013-28; Griffiths C E M et al. (1989) J Am Acad Dermatol 20:617-29; Nickoloff B J et al. (1990) J Invest Dermatol 94:151S-157S; Veale D et al. (1995) Br J Dermatol 132:32-8). Endothelial cells also are hyperproliferative, resulting in angiogenesis and dilation (Detmar M et al. (1994) J Exp Med 180:1141-6; Goodfield M et al. (1994) Br J Dermatol 131:808-13; Malhotra R et al. (1989) Lab Invest 61:162-8; Mordovtsev V N et al. (1989) Am J Dermatopathol 11:33-42) and express increased levels of ICAM-1, E-selectin (CD62E) and vascular cell adhesion molecule-1 (VCAM-1, CD106) (Das P K et al. (1994) Acta Derm Venereol Suppl 186:21-2) as well as MHC class II (Bjerke J R et al. (1988) Acta Derm Venereol 68:306-11). Finally, a mixed leukocytic infiltrate is seen, composed of activated T lymphocytes which produce inflammatory cytokines (Ramirez-Bosca A et al. (1988) Br J Dermatol 119:587-95; Schlaak J F et al. (1994) J Invest Dermatol 102:145-9), neutrophils within the dermis and forming Munro's microabscesses in the epidermis (Christophers E and Sterry W (1993) Psoriasis. In: Dermatology in General Medicine, T B Fitzpatrick, A Z Eisen, K Wolff, I M Freedberg and K F Austen, eds. (New York: McGraw-Hill, Inc.), pp. 489-514), and an increased number of dermal mast cells (Brody I (1986) Ups J Med Sci 91:1-16; Brody I (1984) J Invest Dermatol 82:460-4; Rothe M J et al. (1990) J Am Acad Dermatol 23:615-24; Schubert C et al. (1985) Arch Dermatol Res 277:352-8; Toruniowa B et al. (1988) Arch Dermatol Res 280:189-93; van de Kerkhof P C et al. (1995) Skin Pharmacol 8:25-9).

There is strong evidence that psoriasis is characterized by Th1 and proinflammatory cytokines. IL-2 and IFN-γ are predominant within skin lesions, while IL-4 and IL-10 are scant or absent. Uyemura K et al. (1993) J Invest Dermatol 101:701-5; Schlaak J F et al. (1994) J Invest Dermatol 102:145-9. The proinflammatory cytokines IL-1, IL-6, IL-8, and TNF-α are also present in psoriatic lesions. Ohta Y et al. (1991) Arch Dermatol Res 283:351-6; Lemster B H et al. (1995) Clin Exp Immunol 99:148-54; Nickoloff, B J et al. (1991) Am J Pathol 138:129-40; Ettehadi P et al. (1994) Clin Exp Immunol 96:146-51. Intracutaneous secretion of cytokines is thought to mediate some or all of the tissue alterations seen in psoriasis. These cytokines include TNF-α and IL-1 (Kupper T S (1990) J Clin Invest 86:1783-6); IFN-γ (Barker J N W N et al. (1991) J Dermatol Sci 2:106-11; Gottlieb A B et al. (1988) J Exp Med 167:670-5; Livden J K et al. (1989) Arch Dermatol Res 281:392-7); IL-6 (Castells-Rodellas A et al. (1992) Acta Derm Venereol 72:165-8; Grossman R M et al. (1989) Proc Natl Acad Sci USA 86:6367-71; Neuner P et al. (1991) J Invest Dermatol 97:27-33); IL-8 (Barker J N et al. (1991) Am J Pathol 139:869-76), vascular endothelial growth factor/vascular permeability factor (VEGF/VPF) (Detmar M et al. (1994) J Exp Med 180:1141-6), and TGF-α (Elder J T et al. (1989) Science 243:811-4; Gottlieb A B et al. (1988) J Exp Med 167:670-5; Prinz J C et al. (1994) Eur J Immunol 24:593-8).

Over the past decade, research into the pathophysiology of psoriasis has focused primarily on immunologic mechanisms. Evidence is accumulating that this disease has an immunological basis. However, it has not been convincingly determined if the primary defect that results in psoriasis is an immunologic disorder or resides within the epithelium (Barker J N W N (1994) Bailliere's Clin Rheumatol 8:429-38; Christophers E and Sterry W (1993) Psoriasis. In: Dermatology in General Medicine, T B Fitzpatrick, A Z Eisen, K Wolff, I M Freedberg and K F Austen, eds. (New York: McGraw-Hill, Inc.), pp. 489-514). Abnormal immune regulation is suggested by the frequent association of psoriasis with the expression of certain MHC alleles including HLA B13, B17, Bw57 and Cw6 (Russell T J et al. (1972) N Engl J Med 287:738-40; Tiilikainen A et al. (1980) Br J Dermatol 102:179-84; Watson W et al. (1972) Arch Dermatol 105:197-207; White S H et al. (1972) N Engl J Med 287:740-3), the improvement of psoriatic lesions by treatment with immunosuppressive agents such as cyclosporine A (Ellis C N et al. (1986) JAMA 256:3110-6; Mueller W et al. (1979) N Engl J Med 301:555) and the lymphocyte-specific fusion toxin DAB₃₈₉IL-2 (Gottlieb J L et al. (1995) Nat Med 1:442-7), the possible linkage of a psoriasis susceptibility gene with a gene involved in IL-2 regulation (Tomfohrde J et al. (1994) Science 264:1141-5), and the failure of psoriasis to recur after bone marrow transplantation (Eedy D J et al. (1990) Br Med J 300:908; Jowitt S N et al. (1990) Br Med J 300:1398-9). However, underlying epidermal and/or dermal defects are suggested by altered keratinocyte cell cycle and differentiation (Gelfant S (1982) Cell Tissue Kinet 15:393-7; Weinstein G D et al. (1985) J Invest Dermatol 84:579-83), by aberrant expression of adhesion molecules by keratinocytes and endothelial cells (Das P K et al. (1994) Acta Derm Venereol Suppl 186:21-2; Nickoloff B J et al. (1990) J Invest Dermatol 94:151S-157S; Petzelbauer P et al. (1994) J Invest Dermatol 103:300-5; Veale D et al. (1995) Br J Dermatol 132:32-8; Wakita H et al. (1994) Arch Dermatol 130:457-63), and by the abnormal expression of protooncogenes within keratinocytes (Elder J T et al. (1990) J Invest Dermatol 94:19-25).

Conventional treatments for psoriasis include topical corticosteroids (hydrocortisone, betamethasone, triamcinolone, fluocinolone acetonide, fluocinonide), tar preparations, ultraviolet light (UVB, 295-310 nm, with or without tar; or UVA, 320-400 nm, with or without psoralen), vitamin A analogs (etretinate, acitretin), retinoids (tazarotene), vitamin D analogs (calcipotriol), and immunosuppressive regimens employing potentially toxic agents cyclosporine or methotrexate. Immunosuppressive agents which dampen cell-mediated immunity, including cyclosporine, methotrexate, and lymphocyte-selective toxins, are effective but often are expensive and/or associated with significant risks related to potential side effects.

At least with respect to psoriasis, the foregoing agents, both conventional and experimental, are included among what are herein termed non-allergic inflammatory disease medicaments.

Despite its name, “allergic contact dermatitis” as used herein is a non-allergic inflammatory disease because it involves a cell-mediated, delayed-type hypersensitivity (DTH) reaction rather than an IgE-associated reaction. It is to be distinguished from irritant contact dermatitis which is caused by exposure to substances that directly cause physical, mechanical, or chemical irritation of the skin. Antigens commonly involved in allergic contact dermatitis include plant oleoresins found in poison ivy, poison oak, and poison sumac; certain topical medications, including topical hydrocortisone, topical antibiotics (e.g., neomycin and bacitracin), and benzocaine; nickel in jewelry; fragrances in perfumes, cosmetics, and washing agents; wool alcohols (lanolin); components of rubber (including latex), for example associated with rubber and latex glove use; preservatives (e.g., thimerosal, formaldehyde, quaternium-15); and nail polish. Latex dermatitis, as used herein, refers to delayed-type hypersensitivity involving contact of skin or mucosa with latex. Latex dermatitis occurs commonly but not exclusively in the context of use of latex gloves, for example by health care providers and food service workers. Allergic contact dermatitis in its afferent phase involves presentation of processed antigen by Langerhans cells, professional antigen-presenting cells resident within the dermis, to CD4+T cells. The afferent phase results in the expansion of an antigen-specific T-cell clone that is primed to interact with the triggering antigen upon re-exposure. Upon their encounter with the triggering antigen, sensitized T cells that have migrated to the skin release a cascade of cytokines that leads to inflammation characteristic of allergic contact dermatitis. Conventional medicaments for allergic contact dermatitis include topical corticosteroids, topical antihistamines, and, for severe cases, systemic corticosteroids.

Inflammatory bowel disease, as used herein, includes Crohn's disease and ulcerative colitis. While the precise causes of Crohn's disease and ulcerative colitis remain uncertain, these are well described diseases with partially overlapping but nonetheless distinct clinical and pathologic features. For a review, see Glickman R M, Inflammatory bowel disease: ulcerative colitis and Crohn's disease, in Harrison's Principles of Internal Medicine, 14th Edition, A S Fauci et al. (eds.), New York, McGraw-Hill, 1998. The estimated prevalence of these diseases in the United States is about 70-150 per 100,000 for ulcerative colitis and 20-40 per 100,000 for Crohn's disease.

Ulcerative colitis, which involves primarily the colonic mucosa, with rectal involvement in nearly all cases, is a chronic and recurrent disease clinically characterized by bloody diarrhea and abdominal pain. It may be complicated by anemia, dehydration and electrolyte abnormalities, weight loss, extraintestinal manifestations including arthritis, as well as life-threatening dilation and perforation of the colon. Approximately 25 percent of patients require colectomy at some point in their disease. Patients with longstanding ulcerative colitis are also at increased risk of having or developing cancer of the colon. Characteristically, ulcerative colitis most commonly has uniform, continuous, nontransmural involvement of the colon with loss of surface epithelial cells in involved areas.

Crohn's disease may involve any portion of the gastrointestinal tract, but most commonly it involves the distal small bowel and/or the colon. It too is a chronic disease and, while its symptoms are more variable due to the potential to involve any portion of the gastrointestinal tract, Crohn's disease is more likely than ulcerative colitis to have complications and to require hospitalization. Over two-thirds of patients require surgery at some point in their disease. The bowel inflammation in Crohn's disease characteristically involves the full thickness of the bowel wall. The inflammation extends to involve the mesentery and regional lymph nodes, and this intense inflammatory process results in bowel obstruction in 20 to 30 percent of patients at some point in their disease, as well as fistula and abscess formation in many patients. Also unlike ulcerative colitis, the lesions of Crohn's disease characteristically are discontinuous along the length of the bowel.

Conventional medical approaches to treatment of inflammatory bowel disease include therapy based on sulfasalazine (AZULFIDINE®, Pharmacia & Upjohn), of which 5-aminosalicylic acid is believed to be the active component, and corticosteroids. Related 5-aminosalicylic acid products used in the treatment of ulcerative colitis include olsalazine (DIPENTUM®, Pharmacia & Upjohn) and mesalamine (ROWASA®, Solvay). Corticosteroids include prednisone and prednisolone. Other agents sometimes used in the treatment of ulcerative colitis include immunosuppressive agents cyclosporine A (SANDIMMUNE® and NEORAL®, Novartis), tacrolimus (FK506, PROGRAF®, Fujisawa), and azathioprine (IMURAN®, Faro). At least with respect to inflammatory bowel disease, these agents, as well as experimental agents mentioned elsewhere herein (other than the immunostimulatory nucleic acids of the invention), are included among what are herein termed non-allergic inflammatory disease medicaments.

Th1-like immune activation refers to induction of immune response with a preponderance of Th1 character. For example, Th1-like activation may involve induction of lymphocytes to secrete Th1-like cytokines (e.g., IFN-γ, IL-2, IL-12, IL-18, and TNF) and antibodies (e.g., IgG2a in mice). Th1-like immune activation may also involve activation and/or proliferation of NK cells, CTLs, and macrophages.

A subject in need of Th1-like immune activation is a subject that has or is at risk of developing a disease, disorder, or condition that would benefit from an immune response skewed toward Th1. Such a subject may have or be at risk of having a Th2-mediated disorder that is susceptible to Th1-mediated cross-regulation or suppression. Such disorders include, for example, certain organ-specific autoimmune diseases. Alternatively, such a subject may have or be at risk of having a Th1-deficient state. Such disorders include, for example, tumors, infections with intracellular pathogens, and AIDS. In addition, according to the present invention, such disorders also include non-allergic inflammatory disorders in which further Th1 activation is beneficial in controlling or treating the non-allergic inflammatory disorders.

An immune cell as used herein refers to a cell belonging to the immune system. Such cells include, but are not restricted to, T- and B-lymphocytes, macrophages, monocytes, neutrophils, NK cells, professional antigen-presenting cells, dendritic cells, and their precursors.

The combination of immunostimulatory nucleic acids together with NSAIDs gives a surprising degree of synergy in terms of inducing stronger Th1 responses than would have been expected from additive effects. It has been discovered according to the invention that immunostimulatory nucleic acids such as CpG ODN may cause immune cells to produce Th1 counter-regulatory molecules such as PGE₂ through activating the synthesis of COX-2. NSAIDs inhibit COX-2 activity, removing the negative effects of PGE₂ on CpG-induced immunity. As a result, CpG DNA induces a much stronger Th1-like response in the presence of NSAIDs than in their absence. The compounds do not have to be administered at the same time, and often pre-treatment with NSAIDs may be desirable prior to injection with CpG ODN. NSAID therapy may be continued after CpG therapy for several weeks to several months, depending upon the desired duration of Th1 enhancement. The combination of NSAIDs with CpG therapy is compatible with CpG monotherapy, but also immunization or combinations with monoclonal antibodies, chemotherapy, radiation therapy, and other treatments.

CpG DNA may induce the secretion of Th1 cytokines such as IL-12 and IFN-γ, as well as proinflammatory cytokines including tumor necrosis factor TNF-α, IL-6 and type I IFN. Klinman D M et al. (1996) Proc Natl Acad Sci USA 93:2879-83. In addition, CpG DNA activates NK cells to secrete IFN-γ and enhances their lytic activity. Ballas Z K et al. (1996) J Immunol 157:1840-45; Cowdery J S et al. (1996) J Immunol 156:4570-75; Chace J H et al. (1997) Clin Immunol Immunopathol 84:185-93. These studies demonstrate that CpG DNA is able to induce multiple protein mediators of the immune and inflammatory response.

Prostaglandins (PGs) are lipid mediators that are also key effectors of acute and chronic inflammation. Needleman P et al. (1997) J Rheumatol 24:6-8; Portanova J P et al. (1996) J Exp Med 184:883-91; Anderson G D et al. (1996) J Clin Invest 97:2672-79; Amin A R et al. (1999) Curr Opin Rheumatol 11:202-209; MacDermott R P (1994) Med Clin North Am 78:1207-31. Moreover, PGs are important regulators of cell-mediated immune responses. For example, PGE₂ is a potent inhibitor of Th1-type T cell responses (Betz M et al. (1991) J Immunol 146:108-13), inhibiting IFN-γ production as well as IL-12 and IL-12 receptor expression (Betz M et al. (1991) J Immunol 146:108-13; Wu C Y et al. (1998) J Immunol 161:2723-30). Exogenous PGE₂ is also a potent inhibitor of macrophage-derived inflammatory mediators, including TNF-α production (Kunkel S L et al. (1988) J Biol Chem 263:5380-84) and nitric oxide production (Corraliza I M et al. (1995) Biochem Biophys Res Commun 206:667-73).

Prostaglandins are synthesized from arachidonic acid by prostaglandin G/H synthase, also known as PG endoperoxide synthase and cyclooxygenase (COX). Prostaglandin G/H synthase exists in two isoforms, now termed cyclooxygenase-1 (COX-1) and cyclooxygenase-2 (COX-2). COX-1 is constitutively expressed in cultured endothelial cells and vascular smooth muscle cells. COX-2 is inducibly expressed in response to cytokines, growth factors, phorbol esters, lipopolysaccharide, injury and stress. The generation of other products of the arachidonic acid cascade (besides cyclooxygenase-produced metabolites) is inhibited neither by non-selective nor by COX-2 selective NSAIDs.

There are three broad classes of cyclooxygenase inhibitors: salicylates like aspirin (salicylic acid); nonselective COX inhibitors like indomethacin and other NSAIDs, and selective inhibitors of COX-2 catalytic activity (coxibs).

NSAIDs include, without limitation, diclofenac, diflunisal, fenoprofen, flurbiprofen, ibuprofen, indomethacin, ketoprofen, ketorolac, naproxen, olsalazine, oxaprozin, piroxicam, sulfasalazine, sulindac, tolmetin, salicylates, coxibs, and nitric oxide (NO)-releasing NSAIDs.

Coxibs include, but are not limited to, celecoxib (SC-58635); rofecoxib; valdecoxib; etoricoxib; nimesulide; meloxicam; NS-398; L-745,337; SC236; SC-58125; SC-58236; C-phycocyanin; BMS-279652, BMS-279654, and BMS-279655 (Zhu H et al. (2002) Proc Natl Acad Sci USA 99:3932-37), wogonin (5,7-dihydroxy-8-methoxyflavone; Wakabayashi I et al. (2000) Eur J Pharmacol 406:477-81); and nabumetone.

Inducers of COX-2 expression include TGF-β1 (Sheng H et al. (2000) J Biol Chem 275:6628-35); Ras (Sheng H et al. (1998) J Biol Chem 273:22120-27); IL-1α (Ristimaki A et al. (1994) J Biol Chem 269:11769-75); IL-1β (O'Banion M K et al. (1992) Proc Natl Acad Sci USA 89:4888-92); src oncogene product (Xie W et al. (1991) Proc Natl Acad Sci USA 88:2692-96); peroxynitrite (ONOO⁻; Migita K et al. (2002) Clin Exp Rheumatol 20:59-62); lipopolysaccharide (Lee S H et al. (1992) J Biol Chem 267:25934-38); epidermal growth factor (EGF, Saha D et al. (1999) Neoplasia 1:508-17); and TNF-α (Diaz A et al. (1998) Exp Cell Res 241:222-29).

COX-2 expression inhibitors include wogonin (Wakabayashi I et al. (2000) Eur J Pharmacol 406:477-81); anti-TGF-β1 antibody (Sheng H et al. (2000) J Biol Chem 275:6628-35); dexamethasone (Ristimaki A et al. (1994) J Biol Chem 269:11769-75; Ristimaki A et al. (1996) Biochem J 318:325-31); IL-4, IL-10, and IL-13 (Endo T et al. (1996) J Immunol 156:2240-46); and natriuretic peptides (Kiemer A K et al. (2002) Endocrinology 143:846-52).

At least four subtypes of PGE₂ (E-prostanoid) receptors have been reported. These include EP1, EP2, EP3, and EP4. Stimulation of the EP1 receptor results in activation of phosphatidylinositol hydrolysis and in elevation of intracellular calcium concentration. Funk C D et al. (1993) J Biol Chem 268:26767-72. EP2 and EP4 receptors increase intracellular cAMP concentration through activation of adenylate cyclase. Regan J W et al. (1994) Mol Pharmacol 46:213-20; Bastien L et al. (1994) J Biol Chem 269:11873-77. The EP3 receptor inhibits adenylate cyclase leading to a decrease of cAMP concentration. Furthermore, the EP3 receptor exists as multiple isoforms. Kotani M et al. (1995) Mol Pharmacol 48:869-79; An S et al. (1994) Biochemistry 33:14496-502; Schmid A et al. (1995) J Biochem 228:23-30.

Agents that inhibit PGE₂ signaling through its receptor include, but are not limited to, antibodies specific for PGE₂, antibodies specific for the receptors, antisense nucleic acids specific for the receptors, and small molecule receptor antagonists. As used herein, “agents that inhibit PGE₂ signaling through its receptor” does not include cyclooxygenase inhibitors (described above). Polyclonal and monoclonal antibodies have been raised against PGE₂. Mnich S J et al. (1995) J Immunol 155:4437-44. Antibodies have also been raised against each of the four subtypes of PGE₂ receptors. Morath R et al. 1999) J AM Soc Nephrol 10:1851-60. In addition, a number of EP antagonists have been described, including SC-19220 (EP1), ZM325802 (EP1), AH6809 (EP1 and EP2), and AH23848B (EP4). Sylvia V L et al. (2001) J Steroid Biochem Mol Biol 78:261-74; Santangelo S et al. (2000) J Trauma 48:826-30. AH6809 is 6-isopropoxy-9-oxaxanthene-2-carboxylic acid. AH23848B is [1 alpha(z), 2beta5alpha]-(+/−)-7-[5-[[(1,1′-biphenyl)-4-yl]methoxy]-2-(4-morpholinyl)-3-oxo-cyclopentyl]-4-heptenoic acid.

An “immunostimulatory nucleic acid” as used herein is any nucleic acid containing an immunostimulatory motif or backbone that induces a Th1 immune response and/or suppresses a Th2 immune response. Immunostimulatory motifs include, but are not limited to, CpG motifs, poly-G motifs, and T-rich motifs. In one embodiment immunostimulatory motifs include CpG motifs and T-rich motifs. Immunostimulatory backbones include, but are not limited to, phosphate modified backbones, such as phosphorothioate backbones. Immunostimulatory nucleic acids have been described extensively in the prior art and a brief summary of these nucleic acids is presented below.

The terms “nucleic acid” and “oligonucleotide” are used interchangeably to mean multiple nucleotides (i.e., molecules comprising a sugar (e.g., ribose or deoxyribose) linked to a phosphate group and to an exchangeable organic base, which is either a substituted pyrimidine (e.g., cytosine (C), thymine (T) or uracil (U)) or a substituted purine (e.g., adenine (A) or guanine (G)). As used herein, the terms refer to oligoribonucleotides as well as oligodeoxyribonucleotides. The terms shall also include polynucleosides (i.e., a polynucleotide minus the phosphate) and any other organic base-containing polymer. Nucleic acids include vectors, e.g., plasmids as well as oligonucleotides. Nucleic acid molecules may be obtained from existing nucleic acid sources (e.g., genomic or cDNA), but are preferably synthetic (e.g., produced by oligonucleotide synthesis).

The terms nucleic acid and oligonucleotide also encompass nucleic acids or oligonucleotides with substitutions or modifications, such as in the bases and/or sugars. For example, they include nucleic acids having backbone sugars which are covalently attached to low molecular weight organic groups other than a hydroxyl group at the 3′ position and other than a phosphate group at the 5′ position. Thus modified nucleic acids may include a 2′-O-alkylated ribose group. In addition, modified nucleic acids may include sugars such as arabinose instead of ribose. Thus the nucleic acids may be heterogeneous in backbone composition thereby containing any possible combination of polymer units linked together such as peptide-nucleic acids (which have amino acid backbone with nucleic acid bases). In some embodiments, the nucleic acids are homogeneous in backbone composition. Nucleic acids also include substituted purines and pyrimidines such as C-5 propyne-modified bases. Wagner R W et al. (1996) Nat Biotechnol 14:840-4. Purines and pyrimidines include but are not limited to adenine, cytosine, guanine, thymine, 5-methylcytosine, 2-aminopurine, 2-amino-6-chloropurine, 2,6-diaminopurine, hypoxanthine, and other naturally and non-naturally occurring nucleobases, substituted and unsubstituted aromatic moieties. Other such modifications are well known to those of skill in the art.

Exemplary immunostimulatory nucleic acids as those described herein as well as various control nucleic acids include but are not limited to those presented in Table 1.

TABLE 1
Oligodeoxynucleotide sequences

| SEQ ID NO: | OLIGODEOXYNUCLEOTIDE SEQUENCE | BACKBONE |
|---|---|---|
| 1 | tctcccagcgtgcgccat | s |
| 2 | ataatccagcttgaaccaag | s |
| 3 | ataatcgacgttcaagcaag | s |
| 4 | taccgcgtgcgaccctct | s |
| 5 | ggggagggt | s |
| 6 | ggggagggg | s |
| 7 | ggtgaggtg | s |
| 8 | tccatgtzgttcctgatgct | o |
| 9 | gctaccttagzgtga | o |
| 10 | tccatgazgttcctgatgct | o |
| 11 | tccatgacgttcztgatgct | o |
| 12 | gctagazgttagtgt | o |
| 13 | agctccatggtgctcactg | s |
| 14 | ccacgtcgaccctcaggcga | s |
| 15 | gcacatcgtcccgcagccga | s |
| 16 | gtcactcgtggtacctcga | s |
| 17 | gttggatacaggccagactttgttg | o |
| 18 | gattcaacttgcgctcatcttaggc | o |
| 19 | accatggacgaactgtttcccctc | s |
| 20 | accatggacgagctgtttcccctc | s |
| 21 | accatggacgacctgtttcccctc | s |
| 22 | accatggacgtactgtttcccctc | s |
| 23 | accatggacggtctgtttcccctc | s |
| 24 | accatggacgttctgtttcccctc | s |
| 25 | ccactcacatctgctgctccacaag | o |
| 26 | acttctcatagtccctttggtccag | o |
| 27 | tccatgagcttcctgagtct | o |
| 28 | gaggaaggigiggaigacgt | o |
| 29 | gtgaaticgttcicgggict | o |
| 30 | aaaaaa | s |
| 31 | cccccc | s |
| 32 | ctgtca | s |
| 33 | tcgtag | s |
| 34 | tcgtgg | s |
| 35 | cgtcgt | s |
| 36 | tccatgtcggtcctgagtct | sos |
| 37 | tccatgccggtcctgagtct | sos |
| 38 | tccatgacggtcctgagtct | sos |
| 39 | tccatgtcgatcctgagtct | sos |
| 40 | tccatgtcgctcctgagtct | sos |
| 41 | tccatgtcgttcctgagtct | sos |
| 42 | tccatgacgttcctgagtct | sos |
| 43 | tccataacgttcctgagtct | sos |
| 44 | tccatgacgtccctgagtct | sos |
| 45 | tccatcacgtgcctgagtct | sos |
| 46 | tccatgctggtcctgagtct | sos |
| 47 | tccatgtzggtcctgagtct | sos |
| 48 | ccgcttcctccagatgagctcatgggtttctccaccaag | o |
| 49 | cttggtggagaaacccatgagctcatctggaggaagcgg | o |
| 50 | ccccaaagggatgagaagtt | o |
| 51 | agatagcaaatcggctgacg | o |
| 52 | ggttcacgtgctcatggctg | o |
| 53 | tccatgattttcctgatttt | o |
| 54 | ttgtttttttgtttttttgttttt | s |
| 55 | ttttttttgtttttttgttttt | o |
| 56 | tgctgcttttgtgcttttgtgctt | s |
| 57 | tgctgcttgtgcttttgtgctt | o |
| 58 | gcattcatcaggcgggcaagaat | o |
| 59 | taccgagcttcgacgagatttca | o |
| 60 | gcatgacgttgagct | s |
| 61 | cacgttgaggggcat | s |
| 62 | ctgctgagactggag | s |
| 63 | tccatgacgttcctgacgtt | s |
| 64 | gcatgagcttgagctga | o |
| 65 | tcagcgtgcgcc | s |
| 66 | atgacgttcctgacgtt | s |
| 67 | ttttggggttttggggtttt | s |
| 68 | tctaggctttttaggcttcc | s |
| 69 | tgcattttttaggccaccat | s |
| 70 | tctcccagcgtgcgtgcgccat | s |
| 71 | tctcccagcgggcgcat | s |
| 72 | tctcccagcgagcgccat | s |
| 73 | tctcccagcgcgcgccat | s |
| 74 | ggggtgacgttcagggggg | sos |
| 75 | ggggtccagcgtgcgccatggggg | sos |
| 76 | ggggtgtcgttcagggggg | sos |
| 77 | tccatgtcgttcctgtcgtt | s |
| 78 | tccatagcgttcctagcgtt | s |
| 79 | tcgtcgctgtctccgcttctt | s |
| 80 | gcatgacgttgagct | sos |
| 81 | tctcccagcgtgcgccatat | sos |
| 82 | tccatgazgttcctgazgtt | s |
| 83 | gcatgazgttgagct | o |
| 84 | tccagcgtgcgccata | sos |
| 85 | tctcccagcgtgcgccat | o |
| 86 | gcatgtcgttgagct | sos |
| 87 | tcctgacgttcctgacgtt | s |
| 88 | gcatgatgttgagct | o |
| 89 | gcatttcgaggagct | o |
| 90 | gcatgtagctgagct | o |
| 91 | tccaggacgttcctagttct | o |
| 92 | tccaggagcttcctagttct | o |
| 93 | tccaggatgttcctagttct | o |
| 94 | tccagtctaggcctagttct | o |
| 95 | tccagttcgagcctagttct | o |
| 96 | gcatggcgttgagct | sos |
| 97 | gcatagcgttgagct | sos |
| 98 | gcattgcgttgagct | sos |
| 99 | gcttgcgttgcgttt | sos |
| 100 | tctcccagcgttgcgccatat | sos |
| 101 | tctcccagcgtgcgttatat | sos |
| 102 | tctccctgcgtgcgccatat | sos |
| 103 | tctgcgtgcgtgcgccatat | sos |
| 104 | tctcctagcgtgcgccatat | sos |
| 105 | tctcccagcgtgcgcctttt | sos |
| 106 | gctandcghhagc | o |
| 107 | tcctgacgttccc | o |
| 108 | ggaagacgttaga | o |
| 109 | tcctgacgttaga | o |
| 110 | tcagaccagctggtcgggtgttcctga | o |
| 111 | tcaggaacacccgaccagctggtctga | o |
| 112 | gctagtcgatagc | o |
| 113 | gctagtcgctagc | o |
| 114 | gcttgacgtctagc | o |
| 115 | gcttgacgtttagc | o |
| 116 | gcttgacgtcaagc | o |
| 117 | gctagacgtttagc | o |
| 118 | tccatgacattcctgatgct | o |
| 119 | gctagacgtctagc | o |
| 120 | ggctatgtcgttcctagcc | o |
| 121 | ggctatgtcgatcctagcc | o |
| 122 | ctcatgggtttctccaccaag | o |
| 123 | cttggtggagaaacccatgag | o |
| 124 | tccatgacgttcctagttct | o |
| 125 | ccgcttcctccagatgagctcatg | o |
| 126 | catgagctcatctggaggaagcgg | o |
| 127 | ccagatgagctcatgggtttctcc | o |
| 128 | ggagaaacccatgagctcatctgg | o |
| 129 | agcatcaggaacgacatgga | o |
| 130 | tccatgacgttcctgacgtt | 2ome |
| 131 | gcgcgcgcgcgcgcgcgcg | o |
| 132 | ccggccggccggccggccgg | o |
| 133 | ttccaatcagccccacccgctctggccccaccctcaccctcca | o |
| 134 | tggagggtgagggtggggccagagcgggtggggctgattggaa | o |
| 135 | tcaaatgtgggattttcccatgagtct | o |
| 136 | agactcatgggaaaatcccacatttga | o |
| 137 | tgccaagtgctgagtcactaataaaga | o |
| 138 | tctttattagtgactcagcacttggca | o |
| 139 | tgcaggaagtccgggttttccccaacccccc | o |
| 140 | ggggggttggggaaaacccggacttcctgca | o |
| 141 | ggggactttccgctggggactttccagggggactttcc | sos |
| 142 | tccatgacgttcctctccatgacgttcctctccatgacgttcctc | o |
| 143 | gaggaacgtcatggagaggaacgtcatggagaggaacgtcatgga | o |
| 144 | ataatagagcttcaagcaag | s |
| 145 | tccatgacgttcctgacgtt | s |
| 146 | tccatgacgttcctgacgtt | sos |
| 147 | tccaggactttcctcaggtt | s |
| 148 | tcttgcgatgctaaaggacgtcacattgcacaatcttaataaggt | o |
| 149 | accttattaagattgtgcaatgtgacgtcctttagcatcgcaaga | o |
| 150 | tcctgacgttcctggcggtcctgtcgct | o |
| 151 | tcctgtcgctcctgtcgct | o |
| 152 | tcctgacgttgaagt | o |
| 153 | tcctgtcgttgaagt | o |
| 154 | tcctggcgttgaagt | o |
| 155 | tcctgccgttgaagt | o |
| 156 | tccttacgttgaagt | o |
| 157 | tcctaacgttgaagt | o |
| 158 | tcctcacgttgaagt | o |
| 159 | tcctgacgatgaagt | o |
| 160 | tcctgacgctgaagt | o |
| 161 | tcctgacggtgaagt | o |
| 162 | tcctgacgtagaagt | o |
| 163 | tcctgacgtcgaagt | o |
| 164 | tcctgacgtggaagt | o |
| 165 | tcctgagcttgaagt | o |
| 166 | gggggacgttggggg | o |
| 167 | tcctgacgttccttc | o |
| 168 | tctcccagcgagcgagcgccat | s |
| 169 | tcctgacgttcccctggcggtcccctgtcgct | o |
| 170 | tcctgtcgctcctgtcgctcctgtcgct | o |
| 171 | tcctggcggggaagt | o |
| 172 | tcctgazgttgaagt | o |
| 173 | tcztgacgttgaagt | o |
| 174 | tcctagcgttgaagt | o |
| 175 | tccagacgttgaagt | o |
| 176 | tcctgacggggaagt | o |
| 177 | tcctggcggtgaagt | o |
| 178 | ggctccggggagggaatttttgtctat | o |
| 179 | atagacaaaaattccctccccggagcc | o |
| 180 | tccatgagcttccttgagtct | 2ome |
| 181 | tcgtcgctgtctccgcttctt | so |
| 182 | tcgtcgctgtctccgcttctt | s2o |
| 183 | tcgagacattgcacaatcatctg | o |
| 184 | cagattgtgcaatgtctcga | o |
| 185 | tccatgtcgttcctgatgcg | o |
| 186 | gcgatgtcgttcctgatgct | o |
| 187 | gcgatgtcgttcctgatgcg | o |
| 188 | tccatgtcgttccgcgcgcg | o |
| 189 | tccatgtcgttcctgccgct | o |
| 190 | tccatgtcgttcctgtagct | o |
| 191 | gcggcgggcggcgcgcgccc | o |
| 192 | atcaggaacgtcatgggaagc | o |
| 193 | tccatgagcttcctgagtct | p-ethoxy |
| 194 | tcaacgtt | p-ethoxy |
| 195 | tcaagctt | p-ethoxy |
| 196 | tcctgtcgttcctgtcgtt | s |
| 197 | tccatgtcgtttttgtcgtt | s |
| 198 | tcctgtcgttccttgtcgtt | s |
| 199 | tccttgtcgttcctgtcgtt | s |
| 200 | btccattccatgacgttcctgatgcttcca | os |
| 201 | tcctgtcgttttttgtcgtt | s |
| 202 | tcgtcgctgtctccgcttctt | s |
| 203 | tcgtcgctgtctgcccttctt | s |
| 204 | tcgtcgctgttgtcgtttctt | s |
| 205 | tcctgtcgttcctgtcgttggaacgacagg | o |
| 206 | tcctgtcgttcctgtcgtttcaacgtcaggaacgacagga | o |
| 207 | ggggtctgtcgttttgggggg | sos |
| 208 | ggggtctgtgcttttgggggg | sos |
| 209 | tccggccgttgaagt | o |
| 210 | tccggacggtgaagt | o |
| 211 | tcccgccgttgaagt | o |
| 212 | tccagacggtgaagt | o |
| 213 | tcccgacggtgaagt | o |
| 214 | tccagagcttgaagt | o |
| 215 | tccatgtzgttcctgtzgtt | s |
| 216 | ggggttgacgttttgggggg | sos |
| 217 | tccaggacttctctcaggtt | s |
| 218 | tttttttttttttttttttt | s |
| 219 | tccatgccgttcctgccgtt | s |
| 220 | tccatggcgggcctggcggg | s |
| 221 | tccatgacgttcctgccgtt | s |
| 222 | tccatgacgttcctggcggg | s |
| 223 | tccatgacgttcctgcgttt | s |
| 224 | tccatgacggtcctgacggt | s |
| 225 | tccatgcgtgcgtgcgtttt | s |
| 226 | tccatgcgttgcgttgcgtt | s |
| 227 | btccattccattctaggcctgagtcttccat | os |
| 228 | tccatagcgttcctagcgtt | o |
| 229 | tccatgtcgttcctgtcgtt | o |
| 230 | tccatagcgatcctagcgat | o |
| 231 | tccattgcgttccttgcgtt | o |
| 232 | tccatagcggtcctagcggt | o |
| 233 | tccatgattttcctgcagttcctgatttt | |
| 234 | tccatgacgttcctgcagttcctgacgtt | s |
| 235 | ggcggcggcggcggcggcgg | o |
| 236 | tccacgacgttttcgacgtt | s |
| 237 | tcgtcgttgtcgttgtcgtt | s |
| 238 | tcgtcgttttgtcgttttgtcgtt | s |
| 239 | tcgtcgttgtcgttttgtcgtt | s |
| 240 | gcgtgcgttgtcgttgtcgtt | s |
| 241 | czggczggczgggczccgg | o |
| 242 | gcggcgggeggcgcgcgccc | s |
| 243 | agicccgigaacgiattcac | o |
| 244 | tgtcgtttgtcgtttgtcgtt | s |
| 245 | tgtcgttgtcgttgtcgttgtcgtt | s |
| 246 | tcgtcgtcgtcgtt | s |
| 247 | tgtcgttgtcgtt | s |
| 248 | cccccccccccccccccccc | s |
| 249 | tctagcgtttttagcgttcc | sos |
| 250 | tgcatcccccaggccaccat | s |
| 251 | tcgtcgtcgtcgtcgtcgtcgtt | sos |
| 252 | tcgtcgttgtcgttgtcgtt | sos |
| 253 | tcgtcgttttgtcgttttgtcgtt | sos |
| 254 | tcgtcgttgtcgttttgtcgtt | sos |
| 255 | ggggagggaggaacttcttaaaattcccccagaatgttt | o |
| 256 | aaacattctgggggaattttaagaagttcctccctcccc | o |
| 257 | atgtttacttcttaaaattcccccagaatgttt | o |
| 258 | aaacattctgggggaattttaagaagtaaacat | o |
| 259 | atgtttactagacaaaattcccccagaatgttt | o |
| 260 | aaacattctgggggaattttgtctagtaaacat | o |
| 261 | aaaattgacgttttaaaaaa | sos |
| 262 | ccccttgacgttttcccccc | sos |
| 263 | ttttcgttgtttttgtcgtt | |
| 264 | ctgcagcctgggac | o |
| 265 | acccgtcgtaattatagtaaaaccc | o |
| 266 | ggtacctgtggggacattgtg | o |
| 267 | agczaccgaacgtgagagg | o |
| 268 | tccatgccgttcctgccgtt | o |
| 269 | tccatgacggtcctgacggt | o |
| 270 | tccatgccggtcctgccggt | o |
| 271 | tccatgcgcgtcctgcgcgt | o |
| 272 | ctggtctttctggtttttttctgg | s |
| 273 | tcaggggtggggggaacctt | sos |
| 274 | tccatgazgttcctagttct | o |
| 275 | tccatgatgttcctagttct | o |
| 276 | cccgaagtcatttcctcttaacctgg | o |
| 277 | ccaggttaagaggaaatgacttcggg | o |
| 278 | tcctggzggggaagt | o |
| 279 | gzggzgggzggzgzgzgccc | |
| 280 | tccatgtgcttcctgatgct | o |
| 281 | tccatgtccttcctgatgct | |
| 282 | tccatgtcgttcctagttct | |
| 283 | tccaagtagttcctagttct | o |
| 284 | tccatgtagttcctagttct | o |
| 285 | tcccgcgcgttccgcgcgtt | s |
| 286 | tcctggcggtcctggcggtt | s |
| 287 | tcctggaggggaagt | o |
| 288 | tcctgggggggaagt | o |
| 289 | tcctggtggggaagt | o |
| 290 | tcgtcgttttgtcgttttgtcgtt | o |
| 291 | ctggtctttctggtttttttctgg | o |
| 292 | tccatgacgttcctgacgtt | o |
| 293 | tccaggacttctctcaggtt | sos |
| 294 | tzgtzgttttgtzgttttgtzgtt | o |
| 295 | btczgtcgttttgtcgttttgtcgttttttt | os |
| 296 | gctatgacgttccaaggg | s |
| 297 | tcaacgtt | s |
| 298 | tccaggactttcctcaggtt | o |
| 299 | ctctctgtaggcccgcttgg | s |
| 300 | ctttccgttggacccctggg | s |
| 301 | gtccgggccaggccaaagtc | s |
| 302 | gtgcgcgcgagcccgaaatc | s |
| 303 | tccatgaigttcctgaigtt | s |
| 304 | aatagtcgccataacaaaac | o |
| 305 | aatagtcgccatggcggggc | o |
| 306 | btttttccatgtcgttcctgatgcttttt | os |
| 307 | tcctgtcgttgaagtttttt | o |
| 308 | gctagctttagagctttagagctt | o |
| 309 | tgctgcttcccccccccccc | o |
| 310 | tcgacgttcccccccccccc | o |
| 311 | tcgtcgttcccccccccccc | o |
| 312 | tcgccgttcccccccccccc | o |
| 313 | tcgtcgatcccccccccccc | o |
| 314 | tcctgacgttgaagt | s |
| 315 | tcctgccgttgaagt | s |
| 316 | tcctgacggtgaagt | s |
| 317 | tcctgagcttgaagt | s |
| 318 | tcctggcggggaagt | s |
| 319 | aaaatctgtgcttttaaaaaa | sos |
| 320 | gatccagtcacagtgacctggcagaatctggat | o |
| 321 | gatccagattctgccaggtcactgtgactggat | o |
| 322 | gatccagtcacagtgactcagcagaatctggat | o |
| 323 | gatccagattctgctgagtcactgtgactggat | o |
| 324 | tcgtcgttccccccczcccc | o |
| 325 | tzgtzgttcccccccccccc | o |
| 326 | tzgtcgttcccccccccccc | o |
| 327 | tcgtzgttcccccccccccc | o |
| 328 | tcgtcgctcccccccccccc | o |
| 329 | tcgtcggtcccccccccccc | o |
| 330 | tcggcgttcccccccccccc | o |
| 331 | ggccttttcccccccccccc | o |
| 332 | tcgtcgttttgacgttttgtcgtt | s |
| 333 | tcgtcgttttgacgttttgacgtt | s |
| 334 | ccgtcgttcccccccccccc | o |
| 335 | gcgtcgttcccccccccccc | o |
| 336 | tcgtcattcccccccccccc | o |
| 337 | acgtcgttcccccccccccc | o |
| 338 | ctgtcgttcccccccccccc | o |
| 339 | btttttcgtcgttcccccccccccc | os |
| 340 | tcgtcgttccccccccccccb | o |
| 341 | tcgtcgttttgtcgttttgtcgttb | o |
| 342 | tccagttccttcctcagtct | o |
| 343 | tzgtcgttttgtcgttttgtcgtt | o |
| 344 | tcctggaggggaagt | s |
| 345 | tcctgaaaaggaagt | s |
| 346 | tcgtcgttccccccccc | s |
| 347 | tzgtzgttttgtzgttttgtzgtt | s |
| 348 | ggggtcaagcttgagggggg | sos |
| 349 | tgctgcttcccccccccccc | s |
| 350 | tcgtcgtcgtcgtt | s2 |
| 351 | tcgtcgtcgtcgtt | s2o |
| 352 | tcgtcgtcgtcgtt | os2 |
| 353 | tcaacgttga | s |
| 354 | atagttttccatttttttac | |
| 355 | aatagtcgccatcgcgcgac | o |
| 356 | aatagtcgccatcccgggac | o |
| 357 | aatagtcgccatcccccccc | o |
| 358 | tgctgcttttgtgcttttgtgctt | o |
| 359 | ctgtgctttctgtgtttttctgtg | s |
| 360 | ctaatctttctaatttttttctaa | s |
| 361 | tcgtcgttggtgtcgttggtgtcgtt | s |
| 362 | tcgtcgttggttgtcgttttggtt | s |
| 363 | accatggacgagctgtttcccctc | |
| 364 | tcgtcgttttgcgtgcgttt | s |
| 365 | ctgtaagtgagcttggagag | |
| 366 | gagaacgctggaccttcc | |
| 367 | cgggcgactcagtctatcgg | |
| 368 | gttctcagataaagcggaaccagcaacagacacagaa | |
| 369 | ttctgtgtctgttgctggttccgctttatctgagaac | |
| 370 | cagacacagaagcccgatagacg | |
| 371 | agacagacacgaaacgaccg | |
| 372 | gtctgtcccatgatctcgaa | |
| 373 | gctggccagcttacctcccg | |
| 374 | ggggcctctatacaacctggg | |
| 375 | ggggtccctgagactgcc | |
| 376 | gagaacgctggaccttccat | |
| 377 | tccatgtcggtcctgatgct | |
| 378 | ctcttgcgacctggaaggta | |
| 379 | aggtacagccaggactacga | |
| 380 | accatggacgacctgtttcccctc | |
| 381 | accatggattacctttttcccctt | |
| 382 | atggaaggtccagcgttctc | o |
| 383 | agcatcaggaccgacatgga | o |
| 384 | ctctccaagctcacttacag | |
| 385 | tccctgagactgccccacctt | |
| 386 | gccaccaaaacttgtccatg | |
| 387 | gtccatggcgtgcgggatga | |
| 388 | cctctatacaacctgggac | |
| 389 | gcgctaccggtagcctgagt | |
| 390 | cgactgccgaacaggatatcggtgatcagcactgg | |
| 391 | ccagtgctgatcaccgatatcctgttcggcagtcg | |
| 392 | ccaggttgtatagaggc | |
| 393 | tctcccagcgtacgccat | s |
| 394 | tctcccagcgtgcgtttt | s |
| 395 | tctcccgacgtgcgccat | s |
| 396 | tctcccgtcgtgcgccat | s |
| 397 | ataatcgtcgttcaagcaag | s |
| 398 | tcgtcgttttgtcgttttgtcgt | s2 |
| 399 | tcgtcgttttgtcgttttgtcgtt | s2 |
| 400 | tcntcgtnttntcgtnttntcgtn | s |
| 401 | tctcceagcgtcgccat | s |
| 402 | tctcccatcgtcgccat | s |
| 403 | ataatcgtgcgttcaagaaag | s |
| 404 | ataatcgacgttcccccccc | s |
| 405 | tctatcgacgttcaagcaag | s |
| 406 | tcctgacggggagt | s |
| 407 | tccatgacgttcctgatcc | |
| 408 | tcctggcgtggaagt | s |
| 409 | agcagctttagagctttagagctt | |
| 410 | cccccccccccccccccccccccc | s |
| 411 | tcgtcgttttgtcgttttgtcgttttgtcgtt | s |
| 412 | tcgtcgttttttgtcgttttttgtcgtt | s |
| 413 | tcgtcgtttttttttttttt | s |
| 414 | tttttcaacgttgatttttt | sos |
| 415 | tttttttttttttttttttttttt | s |
| 416 | ggggtcgtcgttttgggggg | |
| 417 | tcgtcgttttgtcgttttgggggg | |
| 418 | tcgtcgctgtctccgcttcttcttgcc | s |
| 419 | tcgtcgctgtctccg | s |
| 420 | ccaggttgtatagaggc | |
| 421 | gctagacgttagcgtga | |
| 422 | ggagctcttcgaacgccata | |
| 423 | tctccatgatggttttatcg | |
| 424 | aaggtggggcagtctcaggga | |
| 425 | atcggaggactggcgcgccg | |
| 426 | ttaggacaaggtctagggtg | |
| 427 | accacaacgagaggaacgca | |
| 428 | ggcagtgcaggctcaccggg | |
| 429 | gaaccttccatgctgtt | |
| 430 | gcttggagggcctgtaagtg | |
| 431 | gtagccttccta | |
| 432 | cggtagccttccta | |
| 433 | cacggtagccttccta | |
| 434 | agcacggtagccttccta | |
| 435 | gaacgctggaccttccat | |
| 436 | gaccttccat | |
| 437 | tggaccttccat | |
| 438 | gctggaccttccat | |
| 439 | acgctggaccttccat | |
| 440 | taagctctgtcaacgccagg | |
| 441 | gagaacgctggaccttccatgt | |
| 442 | ttcatgccttgcaaaatggcg | |
| 443 | tgctagctgtgcctgtacct | |
| 444 | agcatcaggaccgacatgga | |
| 445 | gaccttccatgtcggtcctgat | |
| 446 | acaaccacgagaacgggaac | |
| 447 | gaaccttccatgctgttccg | |
| 448 | caatcaatctgaggagaccc | |
| 449 | tcagctctggtactttttca | |
| 450 | tggttacggtctgtcccatg | |
| 451 | gtctatcggaggactggcgc | |
| 452 | cattttacgggcgggcgggc | |
| 453 | gaggggaccattttacgggc | |
| 454 | tgtccagccgaggggaccat | |
| 455 | cgggcttacggcggatgctg | |
| 456 | tggaccttctatgtcggtcc | |
| 457 | tgtcccatgtttttagaagc | |
| 458 | gtggttacggtcgtgcccat | |
| 459 | cctccaaatgaaagaccccc | |
| 460 | ttgtactctccatgatggtt | |
| 461 | ttccatgctgttccggctgg | |
| 462 | gaccttctatgtcggtcctg | |
| 463 | gagaccgctcgaccttcgat | |
| 464 | ttgccccatattttagaaac | |
| 465 | ttgaaactgaggtgggac | |
| 466 | ctatcggaggactggcgcgcc | |
| 467 | cttggagggcctcccggcgg | |
| 468 | gctgaaccttccatgctgtt | |
| 469 | tagaaacagcattcttcttttagggcagcaca | |
| 470 | agatggttctcagataaagcggaa | |
| 471 | ttccgctttatctgagaaccatct | |
| 472 | gtcccaggttgtatagaggctgc | |
| 473 | gcgccagtcctccgatagac | |
| 474 | ggtctgtcccatatttttag | |
| 475 | tttttcaacgttgagggggg | sos |
| 476 | tttttcaagcgttgatttttt | sos |
| 477 | ggggtcaacgttgatttttt | sos |
| 478 | ggggttttcaacgttttgagggggg | sos |
| 479 | ggttacggtctgtcccatat | |
| 480 | ctgtcccatatttttagaca | |
| 481 | accatcctgaggccattcgg | |
| 482 | cgtctatcgggcttctgtgtctg | |
| 483 | ggccatcccacattgaaagtt | |
| 484 | ccaaatatcggtggtcaagcac | |
| 485 | gtgcttgaccaccgatatttgg | |
| 486 | gtgctgatcaccgatatcctgttcgg | |
| 487 | ggccaactttcaatgtgggatggcctc | |
| 488 | ttccgccgaatggcctcaggatggtac | |
| 489 | tatagtccctgagactgccccaccttctcaacaacc | |
| 490 | gcagcctctatacaacctgggacggga | |
| 491 | ctatcggaggactggcgcgccg | |
| 492 | tatcggaggactggcgcgccg | |
| 493 | gatcggaggactggcgcgccg | |
| 494 | ccgaacaggatatcggtgatcagcac | |
| 495 | ttttggggtcaacgttgagggggg | |
| 496 | ggggtcaacgttgagggggg | sos |
| 497 | cgcgcgcgcgcgcgcgcgcg | s |
| 498 | ggggcatgacgttcgggggg | ss |
| 499 | ggggcatgacgttcaaaaaa | s |
| 500 | ggggcatgagcttcgggggg | s |
| 501 | ggggcatgacgttcgggggg | sos |
| 502 | aaaacatgacgttcaaaaaa | sos |
| 503 | aaaacatgacgttcgggggg | sos |
| 504 | ggggcatgacgttcaaaaaa | sos |
| 505 | accatggacgatctgtttcccctc | s |
| 506 | gccatggacgaactgttccccctc | s |
| 507 | cccccccccccccccccccc | sos |
| 508 | gggggggggggggggggggg | sos |
| 509 | gctgtaaaatgaatcggccg | sos |
| 510 | ttcgggcggactcctccatt | sos |
| 511 | tatgccgcgcccggacttat | sos |
| 512 | ggggtaatcgatcagggggg | sos |
| 513 | tttgagaacgctggaccttc | sos |
| 514 | gatcgctgatctaatgctcg | sos |
| 515 | gtcggtcctgatgctgttcc | sos |
| 516 | tcgtcgtcagttcgctgtcg | sos |
| 517 | ctggaccttccatgtcgg | sos |
| 518 | gctcgttcagcgcgtct | sos |
| 519 | ctggaccttccatgtc | sos |
| 520 | cactgtccttcgtcga | sos |
| 521 | cgctggaccttccatgtcgg | sos |
| 522 | gctgagctcatgccgtctgc | sos |
| 523 | aacgctggaccttccatgtc | sos |
| 524 | tgcatgccgtacacagctct | sos |
| 525 | ccttccatgtcggtcctgat | sos |
| 526 | tactcttcggatcccttgcg | sos |
| 527 | ttccatgtcggtcctgat | sos |
| 528 | ctgattgctctctcgtga | sos |
| 529 | ggcgttattcctgactcgcc | o |
| 530 | cctacgttgtatgcgcccagct | o |
| 531 | ggggtaatcgatgagggggg | o |
| 532 | ttcgggcggactcctccatt | o |
| 534 | gggggttttttttttggggg | o |
| 535 | tttttggggggggggttttt | a |
| 536 | ggggggggggggggggggt | a |
| 537 | aaaaaaaaaaaaaaaaaaaa | o |
| 538 | cccccaaaaaaaaaaccccc | o |
| 539 | aaaaaccccccccccaaaaa | o |
| 540 | tttgaattcaggactggtgaggttgag | o |
| 541 | tttgaatcctcagcggtctccagtggc | o |
| 542 | aattctctatcggggcttctgtgtctgttgctggttccgctttat | o |
| 543 | ctagataaagcggaaccagcaacagacacagaagccccgatagag | o |
| 544 | ttttctagagaggtgcacaatgctctgg | o |
| 545 | tttgaattccgtgtacagaagcgagaagc | o |
| 546 | tttgcggccgctagacttaacctgagagata | o |
| 547 | tttgggcccacgagagacagagacacttc | o |
| 548 | tttgggcccgcttctcgcttctgtacacg | o |
| 549 | gagaacgctggaccttccat | s |
| 550 | tccatgtcggtcctgatgct | s |
| 551 | ctgtcg | s |
| 552 | tcgtga | s |
| 553 | cgtcga | s |
| 554 | agtgct | s |
| 555 | ctgtcg | o |
| 556 | agtgct | o |
| 557 | cgtcga | o |
| 558 | tcgtga | o |
| 559 | gagaacgctccagcttcgat | o |
| 560 | gctagacgtaagcgtga | o |
| 561 | gagaacgctcgaccttccat | o |
| 562 | gagaacgctggacctatccat | o |
| 563 | gctagaggttagcgtga | o |
| 564 | gagaacgctggacttccat | o |
| 565 | tcacgctaacgtctagc | o |
| 566 | bgctagacgttagcgtga | o |
| 567 | atggaaggtcgagcgttctc | o |
| 568 | gagaacgctggaccttcgat | o |
| 569 | gagaacgatggaccttccat | o |
| 570 | gagaacgctggatccat | o |
| 571 | gagaacgctccagcactgat | o |
| 572 | tccatgtcggtcctgctgat | o |
| 573 | atgtcctcggtcctgatgct | o |
| 574 | gagaacgctccaccttccat | o |
| 575 | gagaacgctggaccttcgta | o |
| 576 | batggaaggtccagcgttctc | o |
| 577 | tcctga | o |
| 578 | tcaacgtt | o |
| 579 | aacgtt | o |
| 580 | aacgttga | o |
| 581 | tcacgctaacctctagc | o |
| 582 | gagaacgctggaccttgcat | o |
| 583 | gctggaccttccat | o |
| 584 | gagaacgctggacctcatccat | o |
| 585 | gagaacgctggacgctcatccat | o |
| 586 | aacgttgaggggcat | o |
| 587 | atgcccctcaacgtt | o |
| 588 | tcaacgttga | o |
| 589 | caacgtt | o |
| 590 | acaacgttga | o |
| 591 | tcacgt | o |
| 592 | tcaagctt | o |
| 593 | tcgtca | o |
| 594 | aggatatc | o |
| 595 | tagacgtc | o |
| 596 | gacgtcat | o |
| 597 | ccatcgat | o |
| 598 | atcgatgt | o |
| 599 | atgcatgt | o |
| 600 | ccatgcat | o |
| 601 | agcgctga | o |
| 602 | tcagcgct | o |
| 603 | ccttcgat | o |
| 604 | gtgccggggtctccgggc | s |
| 605 | gctgtggggcggctcctg | s |
| 606 | btcaacgtt | o |
| 607 | ftcaacgtt | o |
| 608 | faacgttga | o |
| 609 | tcaacgt | s |
| 610 | aacgttg | s |
| 611 | cgacga | o |
| 612 | tcgga | o |
| 613 | agaacgtt | o |
| 614 | tcatcgat | o |
| 615 | taaacgtt | s |
| 616 | ccaacgtt | s |
| 617 | gctcga | s |
| 618 | cgacgt | s |
| 619 | acgtgt | s |
| 620 | cgttcg | s |
| 621 | gagcaagctggaccttccat | s |
| 622 | cgcgta | s |
| 623 | cgtacg | s |
| 624 | tcaccggt | s |
| 625 | caagagatgctaacaatgca | s |
| 626 | acccatcaatagctctgtgc | s |
| 627 | tcgacgtc | o |
| 628 | ctagcgct | o |
| 629 | taagcgct | o |
| 630 | tcgcgaattcgcg | o |
| 631 | atggaaggtccagcgttct | o |
| 632 | actggacgttagcgtga | o |
| 633 | cgcctggggctggtctgg | o |
| 634 | gtgtcggggtctccgggc | o |
| 635 | gtgccggggtctccgggc | o |
| 636 | cgccgtcgcggcggttgg | o |
| 637 | gaagttcacgttgaggggcat | o |
| 638 | atctggtgagggcaagctatg | s |
| 639 | gttgaaacccgagaacatcat | s |
| 640 | gcaacgtt | o |
| 641 | gtaacgtt | o |
| 642 | cgaacgtt | o |
| 643 | gaaacgtt | o |
| 644 | caaacgtt | o |
| 645 | ctaacgtt | o |
| 646 | ggaacgtt | o |
| 647 | tgaacgtt | o |
| 648 | acaacgtt | o |
| 649 | ttaacgtt | o |
| 650 | aaaacgtt | o |
| 651 | ataacgtt | o |
| 652 | aacgttct | o |
| 653 | tccgatcg | o |
| 654 | tccgtacg | o |
| 655 | gctagacgctagcgtga | o |
| 656 | gagaacgctggacctcatcatccat | o |
| 657 | gagaacgctagaccttctat | o |
| 658 | actagacgttagtgtga | o |
| 659 | cacaccttggtcaatgtcacgt | o |
| 660 | tctccatcctatggttttatcg | o |
| 661 | cgctggaccttccat | o |
| 662 | caccaccttggtcaatgtcacgt | o |
| 663 | gctagacgttagctgga | o |
| 664 | agtgcgattgcagatcg | o |
| 665 | ttttcgttttgtggttttgtggtt | |
| 666 | ttttcgtttgtcgttttgtcgtt | |
| 667 | tttttgttttgtggttttgtggtt | |
| 668 | accgcatggattctaggcca | s |
| 669 | gctagacgttagcgt | o |
| 670 | aacgctggaccttccat | o |
| 671 | tcaazgtt | o |
| 672 | ccttcgat | o |
| 673 | actagacgttagtgtga | s |
| 674 | gctagaggttagcgtga | s |
| 675 | atggactctccagcgttctc | o |
| 676 | atcgactctcgagcgttctc | o |
| 677 | gctagacgttagc | o |
| 678 | gctagacgt | o |
| 679 | agtgcgattcgagatcg | o |
| 680 | tcagzgct | o |
| 681 | ctgattgctctctcgtga | o |
| 682 | tzaacgtt | o |
| 683 | gagaazgctggaccttccat | o |
| 684 | gctagacgttaggctga | o |
| 685 | gctacttagcgtga | o |
| 686 | gctaccttagcgtga | o |
| 687 | atcgacttcgagcgttctc | o |
| 688 | atgcactctgcagcgttctc | o |
| 689 | agtgactctccagcgttctc | o |
| 690 | gccagatgttagctgga | o |
| 691 | atcgactcgagcgttctc | o |
| 692 | atcgatcgagcgttctc | o |
| 693 | bgagaacgctcgaccttcgat | o |
| 694 | gctagacgttagctgga | sos |
| 695 | atcgactctcgagcgttctc | sos |
| 696 | tagacgttagcgtga | o |
| 697 | cgactctcgagcgttctc | o |
| 698 | ggggtcgaccttggagggggg | sos |
| 699 | gctaacgttagcgtga | o |
| 700 | cgtcgtcgt | o |
| 701 | gagaacgctggaczttccat | o |
| 702 | atcgacctacgtgcgttztc | o |
| 703 | atzgacctacgtgcgttctc | o |
| 704 | gctagazgttagcgt | o |
| 705 | atcgactctcgagzgttctc | o |
| 706 | ggggtaatgcatcagggggg | sos |
| 707 | ggctgtattcctgactgccc | s |
| 708 | ccatgctaacctctagc | o |
| 709 | gctagatgttagcgtga | o |
| 710 | cgtaccttacggtga | o |
| 711 | tccatgctggtcctgatgct | o |
| 712 | atcgactctctcgagcgttctc | o |
| 713 | gctagagcttagcgtga | o |
| 714 | atcgactctcgagtgttctc | o |
| 715 | aacgctcgaccttcgat | o |
| 716 | ctcaacgctggaccttccat | o |
| 717 | atcgacctacgtgcgttctc | o |
| 718 | gagaatgctggaccttccat | o |
| 719 | tcacgctaacctctgac | o |
| 720 | bgagaacgctccagcactgat | o |
| 721 | bgagcaagctggaccttccat | o |
| 722 | cgctagaggttagcgtga | o |
| 723 | gctagatgttaacgt | o |
| 724 | atggaaggtccacgttctc | o |
| 725 | gctagatgttagcgt | o |
| 726 | gctagacgttagtgt | o |
| 727 | tccatgacggtcctgatgct | o |
| 728 | tccatggcggtcctgatgct | o |
| 729 | gctagacgatagcgt | o |
| 730 | gctagtcgatagcgt | o |
| 731 | tccatgacgttcctgatgct | o |
| 732 | tccatgtcgttcctgatgct | o |
| 733 | gctagacgttagzgt | o |
| 734 | gctaggcgttagcgt | o |
| 735 | tccatgtzggtcctgatgct | o |
| 736 | tccatgtcggtzctgatgct | o |
| 737 | atzgactctzgagzgttctc | o |
| 738 | atggaaggtccagtgttctc | o |
| 739 | gcatgacgttgagct | o |
| 740 | ggggtcaacgttgagggggg | s |
| 741 | ggggtcaagtctgagggggg | sos |
| 742 | cgcgcgcgcgcgcgcgcgcg | o |
| 743 | cccccccccccccccccccccccccccc | s |
| 744 | ccccccccccccccccccccccccccccccccccc | s |
| 745 | tccatgtcgctcctgatcct | o |
| 746 | gctaaacgttagcgt | o |
| 747 | tccatgtcgatcctgatgct | o |
| 748 | tccatgccggtcctgatgct | o |
| 749 | aaaatcaacgttgaaaaaaa | sos |
| 750 | tccataacgttcctgatgct | o |
| 751 | tggaggtcccaccgagatcggag | o |
| 752 | cgtcgtcgtcgtcgtcgtcgt | s |
| 753 | ctgctgctgctgctgctgctg | s |
| 754 | gagaacgctccgaccttcgat | s |
| 755 | gctagatgttagcgt | s |
| 756 | gcatgacgttgagct | s |
| 757 | tcaatgctgaf | o |
| 758 | tcaacgttgaf | o |
| 759 | tcaacgttgab | o |
| 760 | gcaatattgcb | o |
| 761 | gcaatattgcf | o |
| 762 | agttgcaact | o |
| 763 | tcttcgaa | o |
| 764 | tcaacgtc | o |
| 765 | ccatgtcggtcctgatgct | o |
| 766 | gtttttatataatttggg | o |
| 767 | tttttgtttgtcgttttgtcgtt | o |
| 768 | ttggggggggtt | s |
| 769 | ggggttgggggtt | s |
| 770 | ggtggtgtaggttttgg | o |
| 771 | bgagaazgctcgaccttcgat | o |
| 772 | tcaacgttaacgttaacgtt | o |
| 773 | bgagcaagztggaccttccat | o |
| 774 | bgagaazgctccagcactgat | o |
| 775 | tcaazgttgab | o |
| 776 | gzaatattgcb | o |
| 777 | tgctgcttttgtcgttttgtgctt | o |
| 778 | ctgcgttagcaatttaactgtg | o |
| 779 | tccatgacgttcctgatgct | s |
| 780 | tgcatgccgtgcatccgtacacagctct | s |
| 781 | tgcatgccgtacacagctct | s |
| 782 | tgcatcagctct | s |
| 783 | tgcgctct | s |
| 784 | cccccccccccc | s |
| 785 | cccccccc | s |
| 786 | tgcatcagctct | sos |
| 787 | tgcatgccgtacacagctct | o |
| 788 | tcaacgttaacgttaacgttaacgttaacgtt | s |
| 789 | gagaacgctcgaccttcgat | s |
| 790 | gtccccatttcccagaggaggaaat | o |
| 791 | ctagcggctgacgtcatcaagctag | o |
| 792 | ctagcttgatgacgtcagccgctag | o |
| 793 | cggctgacgtcatcaa | s |
| 794 | ctgacgtg | o |
| 795 | ctgacgtcat | o |
| 796 | attcgatcggggcggggcgag | o |
| 797 | ctcgccccgccccgatcgaat | o |
| 798 | gactgacgtcagcgt | o |
| 799 | ctagcggctgacgtcataaagctagc | s |
| 800 | ctagctttatgacgtcagccgctagc | s |
| 801 | ctagcggctgagctcataaagctagc | s |
| 802 | ctagtggctgacgtcatcaagctag | s |
| 803 | tccaccacgtggtctatgct | s |
| 804 | gggaatgaaagattttattataag | o |
| 805 | tctaaaaaccatctattcttaaccct | o |
| 806 | agctcaacgtcatgc | o |
| 807 | ttaacggtggtagcggtattggtc | o |
| 808 | ttaagaccaataccgctaccaccg | o |
| 809 | gatctagtgatgagtcagccggatc | o |
| 810 | gatccggctgactcatcactagatc | o |
| 811 | tccaagacgttcctgatgct | o |
| 812 | tccatgacgtccctgatgct | o |
| 813 | tccaccacgtggctgatgct | o |
| 814 | ccacgtggacctctagc | o |
| 815 | tcagaccacgtggtcgggtgttcctga | o |
| 816 | tcaggaacacccgaccacgtggtctga | o |
| 817 | catttccacgatttccca | o |
| 818 | ttcctctctgcaagagact | o |
| 819 | tgtatctctctgaaggact | o |
| 820 | ataaagcgaaactagcagcagtttc | o |
| 821 | gaaactgctgctagtttcgctttat | o |
| 822 | tgcccaaagaggaaaatttgtttcatacag | o |
| 823 | ctgtatgaaacaaattttcctctttgggca | o |
| 824 | ttagggttagggttagggtt | ss |
| 825 | tccatgagcttcctgatgct | ss |
| 826 | aaaacatgacgttcaaaaaa | ss |
| 827 | aaaacatgacgttcgggggg | ss |
| 828 | ggggcatgagcttcgggggg | sos |
| 829 | ctaggctgacgtcatcaagctagt | o |
| 830 | tctgacgtcatctgacgttggctgacgtct | o |
| 831 | ggaattagtaatagatatagaagtt | o |
| 832 | tttaccttttataaacataactaaaacaaa | o |
| 833 | gcgtttttttttgcg | s |
| 834 | atatctaatcaaaacattaacaaa | o |
| 835 | tctatcccaggtggttcctgttag | o |
| 836 | btccatgacgttcctgatgct | o |
| 837 | btccatgagcttcctgatgct | o |
| 838 | tttttttttttttf | o |
| 839 | tttttttttttttf | so |
| 840 | ctagcttgatgagctcagccgctag | o |
| 841 | ttcagttgtcttgctgcttagctaa | o |
| 842 | tccatgagcttcctgagtct | s |
| 843 | ctagcggctgacgtcatcaatctag | o |
| 844 | tgctagctgtgcctgtacct | s |
| 845 | atgctaaaggacgtcacattgca | o |
| 846 | tgcaatgtgacgtcctttagcat | o |
| 847 | gtaggggactttccgagctcgagatcctatg | o |
| 848 | cataggatctcgagctcggaaagtcccctac | o |
| 849 | ctgtcaggaactgcaggtaagg | o |
| 850 | cataacataggaatatttactcctcgc | o |
| 851 | ctccagctccaagaaaggacg | o |
| 852 | gaagtttctggtaagtcttcg | o |
| 853 | tgctgcttttgtgcttttgtgctt | s |
| 854 | tcgtcgttttgtggttttgtggtt | s |
| 855 | tcgtcgtttgtcgttttgtcgtt | s |
| 856 | tcctgacgttcggcgcgcgccc | s |
| 857 | tgctgcttttgtgcttttgtgctt | |
| 858 | tccatgagcttcctgagctt | s |
| 859 | tcgtcgtttcgtcgttttgacgtt | s |
| 860 | tcgtcgtttgcgtgcgtttcgtcgtt | s |
| 861 | tcgcgtgcgttttgtcgttttgacgtt | s |
| 862 | ttcgtcgttttgtcgttttgtcgtt | s |
| 863 | tcctgacggggaagt | s |
| 864 | tcctggcggtgaagt | s |
| 865 | tcctggcgttgaagt | s |
| 866 | tcctgacgtggaagt | s |
| 867 | gcgacgttcggcgcgcgccc | s |
| 868 | gcgacgggcggcgcgcgccc | s |
| 869 | gcggcgtgcggcgcgcgccc | s |
| 870 | gcggcggtcggcgcgcgccc | s |
| 871 | gcgacggtcggcgcgcgccc | s |
| 872 | gcggcgttcggcgcgcgccc | s |
| 873 | gcgacgtgcggcgcgcgccc | s |
| 874 | tgtgggggttttggttttgg | s |
| 875 | aggggaggggaggggagggg | s |
| 876 | tgtgtgtgtgtgtgtgtgtgt | s |
| 877 | ctctctctctctctctctctct | chimeric |
| 878 | ggggtcgacgtcgagggggg | s |
| 879 | atatatatatatatatatatat | s |
| 880 | ttttttttttttttttttttttttttt | s |
| 881 | ttttttttttttttttttttt | s |
| 882 | tttttttttttttttttt | s |
| 883 | gctagaggggagggt | |
| 884 | gctagatgttagggg | |
| 885 | gcatgagggggagct | |
| 886 | atggaaggtccagggggctc | |
| 887 | atggactctggagggggctc | |
| 888 | atggaaggtccaaggggctc | |
| 889 | gagaaggggggaccttggat | |
| 890 | gagaaggggggaccttccat | |
| 891 | gagaaggggccagcactgat | |
| 892 | tccatgtggggcctgatgct | |
| 893 | tccatgaggggcctgatgct | |
| 894 | tccatgtggggcctgctgat | |
| 895 | atggactctccggggttctc | |
| 896 | atggaaggtccggggttctc | |
| 897 | atggactctggaggggtctc | |
| 898 | atggaggctccatggggctc | |
| 899 | atggactctggggggttctc | |
| 900 | tccatgtgggtggggatgct | |
| 901 | tccatgcgggtggggatgct | |
| 902 | tccatgggggtcctgatgct | |
| 903 | tccatggggtccctgatgct | |
| 904 | tccatggggtgcctgatgct | |
| 905 | tccatggggttcctgatgct | |
| 906 | tccatcgggggcctgatgct | |
| 907 | gctagagggagtgt | |
| 908 | gmggtcaacgttgagggmggg | s |
| 909 | ggggagttcgttgaggggggg | s |
| 910 | tcgtcgtttccccccccccc | s |
| 911 | ttggggggttttttttttttttttt | s |
| 912 | tttaaattttaaaatttaaaata | s |
| 913 | ttggtttttttggtttttttttgg | s |
| 914 | tttcccttttccccttttcccctc | s |
| 915 | ggggtcatcgatgaggggggs | sos |
| 916 | tccatgacgttcctgacgtt | |
| 917 | gggggacgatcgtcggggg | sos |
| 918 | gggggtcgtacgacgggggg | sos |
| 919 | tttttttttttttttttttttttt | po |
| 920 | aaaaaaaaaaaaaaaaaaaaaaaa | po |
| 921 | cccccccccccccccccccccccc | po |
| 922 | tcgtcgttttgtcgttttgtcgtt | |
| 923 | ggggtcaacgttgagggggg | |
| 924 | ggggtcaagcttgagggggg | |
| 925 | tgctgcttcccccccccccc | |
| 926 | ggggacgtcgacgtgggggg | sos |
| 927 | ggggtcgtcgacgagggggg | sos |
| 928 | ggggtcgacgtacgtcgagggggg | sos |
| 929 | ggggaccggtaccggtgggggg | sos |
| 930 | gggtcgacgtcgagggggg | sos |
| 931 | ggggtcgacgtcgaggggg | sos |
| 932 | ggggaacgttaacgttgggggg | sos |
| 933 | ggggtcaccggtgagggggg | sos |
| 934 | ggggtcgttcgaacgagggggg | sos |
| 935 | ggggacgttcgaacgtgggggg | sos |
| 936 | tcaactttga | s |
| 937 | tcaagcttga | s |
| 938 | tcacgatcgtga | s |
| 939 | tcagcatgctga | s |
| 940 | gggggagcatgctggggggg | sos |
| 941 | gggggacgatatcgtcgggggg | sos |
| 942 | gggggacgacgtcgtcgggggg | sos |
| 943 | gggggacgagctcgtcgggggg | sos |
| 944 | gggggacgtacgtcgggggg | sos |
| 945 | tcaacgtt | |
| 946 | tccataccggtcctgatgct | |
| 947 | tccataccggtcctaccggt | s |
| 948 | gggggacgatcgttgggggg | sos |
| 949 | ggggaacgatcgtcgggggg | sos |
| 950 | ggggggacgatcgtcgggggg | sos |
| 951 | gggggacgatcgtcggggggg | sos |
| 952 | aaagacgttaaa | po |
| 953 | aaagagcttaaa | po |
| 954 | aaagazgttaaa | po |
| 955 | aaattcggaaaa | po |
| 956 | gggggtcatcgatgagggggg | sos |
| 957 | gggggtcaacgttgagggggg | sos |
| 958 | atgtagcttaataacaaagc | po |
| 959 | ggatcccttgagttacttct | po |
| 960 | ccattccacttctgattacc | po |
| 961 | tatgtattatcatgtagata | po |
| 962 | agcctacgtattcaccctcc | po |
| 963 | ttcctgcaactactattgta | po |
| 964 | atagaaggccctacaccagt | po |
| 965 | ttacaccggtctatggaggt | po |
| 966 | ctaaccagatcaagtctagg | po |
| 967 | cctagacttgatctggttag | po |
| 968 | tataagcctcgtccgacatg | po |
| 969 | catgtcggacgaggcttata | po |
| 970 | tggtggtggggagtaagctc | po |
| 971 | gagctactcccccaccacca | po |
| 972 | gccttcgatcttcgttggga | po |
| 973 | tggacttctctttgccgtct | po |
| 974 | atgctgtagcccagcgataa | po |
| 975 | accgaatcagcggaaagtga | po |
| 976 | ggagaaacccatgagctcatctgg | |
| 977 | accacagaccagcaggcaga | |
| 978 | gagcgtgaactgcgcgaaga | |
| 979 | tcggtacccttgcagcggtt | |
| 980 | ctggagccctagccaaggat | |
| 981 | gcgactccatcaccagcgat | |
| 982 | cctgaagtaagaaccagatgt | |
| 983 | ctgtgttatctgacatacacc | |
| 984 | aattagccttaggtgattggg | |
| 985 | acatctggttcttacttcagg | |
| 986 | ataagtcatattttgggaactac | |
| 987 | cccaatcacctaaggctaatt | |
| 988 | tcctggcgzggaagt | s |
| 989 | ggggaacgacgtcgttgggggg | sos |
| 990 | ggggaacgtacgtcgggggg | sos |
| 991 | ggggaacgtacgtacgttgggggg | sos |
| 992 | ggggtcgacgtcgagggg | sos |
| 993 | ggggacgtcgacgtggggg | sos |
| 994 | gcactcttcgaagctacagccggcagcctctgat | |
| 995 | cggctcttccatgaggtctttgctaatcttgg | |
| 996 | cggctcttccatgaaagtctttggacgatgtgagc | |
| 997 | tcctgcaggttaagt | s |
| 998 | gggggtcgttcgttgggggg | sos |
| 999 | gggggatgattgttgggggg | sos |
| 1000 | gggggazgatzgttgggggg | sos |
| 1001 | gggggagctagcttgggggg | sos |
| 1002 | ggttcttttggtccttgtct | s |
| 1003 | ggttcttttggtcctcgtct | s |
| 1004 | ggttcttttggtccttatct | s |
| 1005 | ggttcttggtttccttgtct | s |
| 1006 | tggtcttttggtccttgtct | s |
| 1007 | ggttcaaatggtccttgtct | s |
| 1008 | gggtcttttgggccttgtct | s |
| 1009 | tccaggacttctctcaggtttttt | s |
| 1010 | tccaaaacttctctcaaatt | s |
| 1011 | tactacttttatacttttatactt | s |
| 1012 | tgtgtgtgtgtgtgtgtgtgtgtg | s |
| 1013 | ttgttgttgttgtttgttgttgttg | s |
| 1014 | ggctccggggagggaatttttgtctat | s |
| 1015 | gggacgatcgtcggggggg | sos |
| 1016 | gggtcgtcgacgaggggggg | sos |
| 1017 | ggtcgtcgacgaggggggg | sos |
| 1018 | gggtcgtcgtcgtggggggg | sos |
| 1019 | ggggacgatcgtcggggggg | sos |
| 1020 | ggggacgtcgtcgtgggggg | sos |
| 1021 | ggggtcgacgtcgacgtcgaggggggg | sos |
| 1022 | ggggaaccgcggttggggggg | sos |
| 1023 | ggggacgacgtcgtggggggg | sos |
| 1024 | tcgtcgtcgtcgtcgtggggggg | sos |
| 1q25 | tcctgccggggaagt | s |
| 1026 | tcctgcaggggaagt | s |
| 1027 | tcctgaaggggaagt | s |
| 1028 | tcctggcgggcaagt | s |
| 1029 | tcctggcgggtaagt | s |
| 1030 | tcctggcgggaaagt | s |
| 1031 | tccgggcggggaagt | s |
| 1032 | tcggggcggggaagt | s |
| 1033 | tcccggcggggaagt | s |
| 1034 | gggggacgttggggg | s |
| 1035 | ggggttttttttttgggggg | sos |
| 1036 | ggggccccccccccgggggg | sos |
| 1037 | ggggttgttgttgttgggggg | sos |

In Table 1 with respect to sequences the letter symbols aside from a, c, t, and g are defined as follows:

“b” indicates a biotin moiety attached to that end of the oligonucleotide when it is single and is listed on the 5′ or 3′ end of oligonucleotide;

“d” represents a, g, or t;

“f” represents fluorescein isothiocyanate (FITC) moiety attached to the 5′ or 3′ end of oligonucleotide;

“h” represents a, c, or t;

“i” represents inosine;

“m” represents a or c;

“n” represents any nucleotide;

“s” represents c or g;

“z” represents 5-methylcytosine.

Also in Table 1 with respect to backbones the notations are defined as follows:

“o” represents phosphodiester;

“os” represents phosphorothioate and phosphodiester chimeric with phosphodiester on 5′ end;

“os2” represents phosphorodithioate and phosphodiester chimeric with phosphodiester on 5′ end;

“p-ethoxy” represents p-ethoxy backbone (see, e.g., U.S. Pat. No. 6,015,886);

“po” represents phosphodiester;

“2ome” represents 2′-OMe, 2′-O-methoxy;

“s” represents phosphorothioate;

“s2” represents phosphorodithioate;

“s2o” represents phosphorodithioate and phosphodiester chimeric with phosphodiester on 3′ end;

“so” represents phosphorothioate and phosphodiester chimeric with phosphodiester on 3′ end;

“sos” represents chimeric phosphorothioate/phosphodiester with phosphorothioate at the 5′ and 3′ ends; and

“ss” represents phosphorodithioate.

In some aspects of the invention an isolated immunostimulatory nucleic acid is administered alone for the treatment of non-allergic inflammatory disease.

In some embodiments, the immunostimulatory nucleic acid is a CpG nucleic acid. CpG sequences, while relatively rare in human DNA are commonly found in the DNA of infectious organisms such as bacteria. The human immune system has apparently evolved to recognize CpG sequences as an early warning sign of infection and to initiate an immediate and powerful immune response against invading pathogens without causing adverse reactions frequently seen with other immune stimulatory agents. Thus CpG-containing nucleic acids, relying on this innate immune defense mechanism, can utilize a unique and natural pathway for immune therapy. The effects of CpG nucleic acids on immune modulation have been described extensively in PCT published patent applications, such as WO 96/02555, WO 96/18810; WO 98/37919; WO 98/40100; WO 98/52581; WO 99/51259; and WO 99/56755. The entire contents of each of these patent applications is hereby incorporated by reference.

A CpG nucleic acid is a nucleic acid which includes at least one unmethylated CpG dinucleotide. A nucleic acid containing at least one unmethylated CpG dinucleotide is a nucleic acid molecule which contains an unmethylated cytosine in a cytosine-guanine dinucleotide sequence (i.e., “CpG DNA” or DNA containing a 5′ cytosine followed by 3′ guanine and linked by a phosphate bond) and activates the immune system. The CpG nucleic acids can be double-stranded or single-stranded. Generally, double-stranded molecules are more stable in vivo, while single-stranded molecules have increased immune activity. Thus in some aspects of the invention it is preferred that the nucleic acid be single-stranded and in other aspects it is preferred that the nucleic acid be double-stranded. The terms CpG nucleic acid or CpG oligonucleotide as used herein refer to an immunostimulatory CpG nucleic acid or a nucleic acid unless otherwise indicated. The entire immunostimulatory nucleic acid can be unmethylated or portions may be unmethylated but at least the C of the 5′-CG-3′ must be unmethylated.

In one preferred embodiment the invention provides an immunostimulatory nucleic acid which is a CpG nucleic acid represented by at least the formula:

5′-X₁X₂CGX₃X₄-3′

wherein X₁, X₂, X₃, and X₄ are nucleotides. In one embodiment X₂ is adenine, guanine, cytosine, or thymine. In another embodiment X₃ is cytosine, guanine, adenine, or thymine. In other embodiments X₂ is adenine, guanine, or thymine and X₃ is cytosine, adenine, or thymine.

In another embodiment the immunostimulatory nucleic acid is an isolated CpG nucleic acid represented by at least the formula:

5′-N₁X₁X₂CGX₃X₄N₂-3′

wherein X₁, X₂, X₃, and X₄ are nucleotides, N is any nucleotide, and N₁ and N₂ are nucleic acid sequences composed of from about 0-25 nucleotides each. In one embodiment X₁X₂ are nucleotides selected from the group consisting of: GpT, GpG, GpA, ApA, ApT, ApG, CpT, CpA, CpG, TpA, TpT, and TpG; and X₃X₄ are nucleotides selected from the group consisting of: TpT, ApT, TpG, ApG, CpG, TpC, ApC, CpC, TpA, ApA, and CpA. Preferably X₁X₂ are GpA or GpT and X₃X₄ are TpT. In other embodiments X₁ or X₂ or both are purines and X₃ or X₄ or both are pyrimidines or X₁X₂ are GpA and X₃ or X₄ or both are pyrimidines. In another preferred embodiment X₁X₂ are nucleotides selected from the group consisting of: TpA, ApA, ApC, ApG, and GpG. In yet another embodiment X₃X₄ are nucleotides selected from the group consisting of: TpT, TpA, TpG, ApA, ApG, ApC, and CpA. X₁X₂ in another embodiment are nucleotides selected from the group consisting of: TpT, TpG, ApT, GpC, CpC, CpT, TpC, GpT and CpG.

In another preferred embodiment the immunostimulatory nucleic acid has the sequence 5′-TCN₁TX₁X₂CGX₃X₄-3′. The immunostimulatory nucleic acids of the invention in some embodiments include X₁X₂ selected from the group consisting of GpT, GpG, GpA and ApA and X₃X₄ is selected from the group consisting of TpT, CpT and TpC.

In some embodiments, the CpG oligonucleotide has a sequence selected from the group consisting of SEQ ID NO: 1, 3, 4, 14-16, 18-24, 28, 29, 33-45, 48, 49, 51, 52, 58, 61, 63, 65, 66, 70-81, 84-87, 89, 91, 95-117, 119-121, 124-126, 129-134, 139-143, 145, 146, 148-164, 166-171, 173-179, 181-191, 194, 196-207, 209-213, 216, 219-232, 234-247, 249, 251-256, 261-263, 265, 267-271, 276, 277, 282, 285, 286, 290, 292, 295-297, 299-302, 304-307, 310-316, 318, 324, 326-330, 332-341, 343, 346, 350-353, 355-357, 361-364, 366-373, 376-380, 382, 383, 387, 389-391, 393-408, 411-414, 416, 417, 421-423, 425, 427, 428, 432-435, 439-442, 444-447, 450-453, 455, 456, 458, 461-463, 466, 467, 470, 471, 473-479, 481, 482, 484-486, 488, 490-506, 509-518, 520-532, 541-543, 545-553, 555, 557, 576, 578-582, 584-591, 593, 595-598, 601-620, 622-624, 627-637, 639-670, 672-679, 681, 682, 684-689, 691-705, 709, 710, 712-717, 719, 720, 722-734, 736, 739-740, 742, 745-752, 754-756, 758, 759, 763-765, 771, 772, 777-781, 783, 787-789, 791-803, 806-817, 820, 821, 826-830, 833, 836, 843, 845-848, 850-852, 854-856, 859-873, and 878.

“Palindromic sequence” shall mean an inverted repeat (i.e., a sequence such as ABCDEE′D′C′B′A′ in which A and A′, B and B′, etc., are bases capable of forming the usual Watson-Crick base pairs. In vivo, such sequences may form double-stranded structures. In one embodiment the CpG nucleic acid contains a palindromic sequence. A palindromic sequence used in this context refers to a palindrome in which the CpG is part of the palindrome. In some embodiments the CpG is the center of the palindrome. In another embodiment the CpG nucleic acid is free of a palindrome. An immunostimulatory nucleic acid that is free of a palindrome is one in which the CpG dinucleotide is not part of a palindrome. Such an oligonucleotide may include a palindrome in which the CpG is not the center of the palindrome.

The CpG nucleic acid sequences of the invention are those broadly described above as well as disclosed in PCT published patent applications WO 96/02555 and WO 98/18810 claiming priority to U.S. Pat. Nos. 6,194,388 B1 and 6,239,116 B1.

In certain preferred embodiments, the CpG nucleic acid is particularly potent as an inducer of type I IFN, i.e., IFN-α or IFN-β. A CpG nucleic acid of this type includes an oligonucleotide having a phosphate modification at the 5′ and 3′ ends of the molecule with a phosphodiester central region. This preferred molecule is exemplified by the following formula:

5′-Y₁N₁CGN₂Y₂-3′

wherein Y₁ and Y₂ are, independent of one another, nucleic acid molecules having between 1 and 10 nucleotides, and wherein Y₁ includes at least one modified internucleotide linkage and Y₂ includes at least one modified internucleotide linkage and wherein N₁ and N₂ are nucleic acid molecules, each independent of one another having between 0 and 20 nucleotides and in some embodiments, between 3 and 8 nucleotides, but wherein N₁CGN₂ has at least 6 nucleotides in total and wherein the nucleotides of N₁CGN₂ have a phosphodiester backbone. Oligonucleotides having one or more phosphorothioate-modified internucleotide linkages with a central region having one or more phosphodiester internucleotide linkages demonstrated unexpectedly high ability to induce IFN-α. The activity of these oligonucleotides was particularly high when the first two and last five internucleotide linkages included phosphate modifications and/or the oligonucleotide included poly-G ends, and the oligonucleotide included a palindrome.

Y₁ and Y₂ are considered independent of one another. This means that each of Y₁ and Y₂ may or may not have different sequences and different backbone linkages from one another in the same molecule. The sequences vary, but in some cases Y₁ and Y₂ have a poly-G sequence. A poly-G sequence in this context refers in some embodiments to at least 2 Gs in a row. In more preferred embodiments, a poly-G sequence in this context refers to at least 3 Gs in a row. In other embodiments the poly-G sequence in this context refers to at least 4, 5, 6, 7, or 8.

In some embodiments Y₁ and Y₂ have between 3 and 8 or between 4 and 7 nucleotides. At least one of these nucleotides includes a modified internucleotide linkage. In some embodiments Y₁ and Y₂ include at least two modified internucleotide linkages, and in other embodiments Y₁ and Y₂ include between two and five modified internucleotide linkages. In yet other embodiments Y₁ has two modified internucleotide linkages and Y₂ has five modified internucleotide linkages. In other embodiments Y₁ has five modified internucleotide linkages and Y₂ has two modified internucleotide linkages.

Exemplary preferred immunostimulatory nucleic acids of the invention for inducing secretion of type I IFN are shown in Table 2 below, in which lower case letters indicate phosphorothioate linkages and upper case letters indicate phosphodiester linkages.

TABLE 2
Exemplary preferred immunostimulatory
nucleic IFN acids for inducing Type I

| SEQ ID NO: | OLIGODEOXYNUCLEOTIDE SEQUENCE |
|---|---|
| 253 | tcGTCGTTTTGTCGTTTTgtcgtT |
| 416 | ggGGTCGTCGTTTTgggggG |
| 417 | tcGTCGTTTTGTCGTTTTgggggG |
| 496 | ggGGTCAACGTTGAgggggG |
| 918 | ggGGGTCGTACGACgggggG |
| 927 | ggGGTCGTCGACGAgggggG |
| 928 | ggGGTCGACGTACGTCGAgggggG |
| 929 | ggGGACCGGTACCGGTgggggG |
| 930 | ggGTCGACGTCGAgggggG |
| 932 | ggGGAACGTTAACGTTgggggG |
| 933 | ggGGTCACCGGTGAgggggG |
| 934 | ggGGTCGTTCGAACGAgggggG |
| 935 | ggGGACGTTCGAACGTgggggG |
| 941 | ggGGGACGATATCGTCgggggG |
| 942 | ggGGGACGACGTCGTCgggggG |
| 943 | ggGGGACGAGCTCGTCgggggG |
| 944 | ggGGGACGTACGTCgggggG |
| 948 | ggGGGACGATCGTTGggggG |
| 949 | ggGGAACGATCCTCgggggG |
| 950 | ggGGGGACGATCGTCgggggG |
| 951 | ggGGGACGATCGTCGgggggG |
| 956 | ggGGGTCATCGATGAgggggG |
| 989 | ggGGAACGACGTCGTTgggggG |
| 990 | ggGGAACGTACGTCgggggG |
| 991 | ggGGAACGTACGTACGTTgggggG |
| 992 | ggGGTCGACGTCGagggG |
| 993 | ggGGACGTCGACGTggggG |
| 998 | ggGGGTCGTTCGTTgggggG |
| 1015 | ggGACGATCGTCGgggggG |
| 1016 | ggGTCGTCGACGAggggggG |
| 1017 | ggTCGTCGACGAGgggggG |
| 1020 | ggGGACGATCGTCGgggggG |
| 1021 | ggGGTCGACGTCGACGTCGAGgggggG |
| 1023 | ggGGACGACGTCGTGgggggG |
| 1038 | ggGGTCATCGATGAgggggG |
| 1039 | ggGGGACGATCGTCgggggG |
| 1040 | ggGGTCGACGTCGAgggggG |

In some embodiments of the invention the immunostimulatory nucleic acids include methylated CpG dinucleotides. If the total length of a CpG immunostimulatory nucleic acid in 20 nucleotide residues or less, then CpG motifs are important in determining the immune effect of the nucleic acid, and methylation of these motifs reduces the potency of the immunostimulatory effects of the nucleic acid. If the length of the immunostimulatory nucleic acid is increased to 24, then the immunostimulatory effects of the nucleic acid become less dependent on the CpG motifs, and are no longer abolished by methylation of the CpG motifs.

A methylated CpG nucleic acid is a nucleic acid which includes at least one methylated CpG dinucleotide, i.e., is a nucleic acid molecule which contains a methylated cytosine in a cytosine-guanine dinucleotide sequence (e.g., DNA containing a 5′5-methylcytosine followed by 3′ guanine linked by a phosphate bond) and activates the immune system. The methylated CpG nucleic acids can be double-stranded or single-stranded.

In some embodiments, the methylated CpG oligonucleotide has a sequence selected from the group consisting of SEQ ID NO:8-10, 12, 47, 82, 83, 172, 215, 241, 274, 278, 279, 294, 325-327 343, 347, 671, 680, 682, 703-705, 733, 735, 737, 771, 774, 775, 954, and 1000. In some embodiments the methylated CpG oligonucleotide has a sequence identical to any CpG oligonucleotide, including but not limited to those disclosed herein, with the substitution of 5-methylcytosine for cytosine in the 5′-CG-3′ CpG motif. Preferably the methylated CpG oligonucleotide is at least 20 nucleotides long, and more preferably at least 24 nucleotides long.

The immunostimulatory nucleic acids of the invention also include nucleic acids having T-rich motifs. As used herein, a “T-rich nucleic acid” is a nucleic acid which includes at least one poly-T sequence and/or which has a nucleotide composition of greater than 25% T nucleotide residues. A nucleic acid having a poly-T sequence includes at least four Ts in a row, such as 5′-TTTT-3′. Preferably the T-rich nucleic acid includes more than one poly-T sequence. In preferred embodiments the T-rich nucleic acid may have 2, 3, 4, etc., poly-T sequences. One of the most highly immunostimulatory T-rich oligonucleotides is a nucleic acid composed entirely of T nucleotide residues. Other T-rich nucleic acids have a nucleotide composition of greater than 25% T nucleotide residues, but do not necessarily include a poly-T sequence. In these T-rich nucleic acids the T nucleotide residues may be separated from one another by other types of nucleotide residues, i.e., G, C, and A. In some embodiments the T-rich nucleic acids have a nucleotide composition of greater than 35%, 40%, 50%, 60%, 70%, 80%, 90%, and 99%, T nucleotide residues and every integer % in between. Preferably the T-rich nucleic acids have at least one poly-T sequence and a nucleotide composition of greater than 25% T nucleotide residues.

Aside from their own immune effect, the presence of poly-T sequences or a T-rich nucleic acid enhances the therapeutic immune effect of other motifs, such as CpG motifs or their mimics.

In one embodiment the T-rich nucleic acid is represented by at least the formula:

5′-X₁X₂TTTTX₃X₄-3′

wherein X₁, X₂, X₃, and X₄ are nucleotides. In one embodiment X₁X₂ is TT and/or X₃X₄ is TT. In another embodiment X₁X₂ are any one of the following dinucleotides: TA, TG, TC, AT, AA, AG, AC, CT, CC, CA, GT, GG, GA, and GC; and X₃X₄ are any one of the following dinucleotides: TA, TG, TC, AT, AA, AG, AC, CT, CC, CA, GT, GG, GA, and GC.

T-rich nucleic acids are also described and claimed in U.S. patent application Ser. No. 09/669,187 filed on Sep. 25, 2000, claiming priority to U.S. Provisional Patent Application No. 60/156,113 filed on Sep. 25, 1999, which is hereby incorporated by reference. Many of the immunostimulatory ODN presented in Table 1 are T-rich nucleic acids as defined here.

Poly-G-containing nucleic acids may also be immunostimulatory. PCT published patent application number WO 00/14217, which claims priority to European Patent Application No. 98116652.3, filed on Sep. 3, 1998, describes poly-G-containing oligonucleotides and their uses. A variety of other references also describe the immunostimulatory properties of poly-G nucleic acids, including Pisetsky D S et al. (1993) Mol Biol Reports 18:217-21; Krieger M et al. (1994) Ann Rev Biochem 63:601-37; Macaya R F et al. (1993) Proc Natl Acad Sci USA 90:3745-9; Wyatt J R et al. (1994) Proc Natl Acad Sci USA 91:1356-60; Rando and Hogan (1998) In: Applied Antisense Oligonucleotide Technology, ed. Krieg and Stein, pp. 335-352; and Kimura Y et al. (1994) J Biochem (Japan) 116:991-4.

In some aspects of the invention the poly-G-containing nucleic acids are administered alone for the treatment of non-allergic inflammatory disease. It was previously suggested in the prior art that oligo(dG)₂₀ oligonucleotides having phosphorothioate, but not phosphodiester, backbones inhibit the production of interferon gamma (IFN-γ) by splenocytes stimulated in vitro with compounds such as concanavalin A, bacterial DNA, or the combination of phorbol myristate acetate (PMA) and the calcium ionophore A23187. Halpern M D et al. (1995) Immunopharmacology 29:47-52. This effect was interpreted by Halpern et al. to be independent of any direct antisense mechanism. Burgess and co-workers also described a sequence-specific but non-antisense antiproliferative effect on smooth muscle cells of phosphorothioate oligonucleotides containing at least four consecutive guanosine residues (G4) or at least two sets of three consecutive guanosine residues (2×G3). Burgess T L et al. (1995) Proc Natl Acad Sci USA 92:4051-5. It has also been reported that particular phosphodiester poly-G oligonucleotides inhibit the binding of IFN-γ to its receptor, which prevents the normal enhancement of major histocompatibility complex (MHC) Class I and intercellular adhesion molecule (ICAM)-1 in response to IFN-γ. Ramanathan M et al. (1994) Transplantation 57:612-15. Finally, Yaswen and co-workers reported that G4-containing phosphorothioate oligonucleotides were antiproliferative in some cultured mammary epithelial cells but not in others. Yaswen P et al. (1993) Antisense Res Dev 3:67-77.

It was surprisingly discovered, according to the invention, that poly-G nucleic acids are useful for treating or preventing non-allergic inflammatory disease. Thus, in this aspect of the invention, poly-G nucleic acids are administered alone or optionally with other non-allergic inflammatory disease medicaments for the treatment of non-allergic inflammatory disease.

Poly-G nucleic acids preferably are nucleic acids having the following formula:

5′-X₁X₂GGGX₃X₄-3′

wherein X₁, X₂, X₃, and X₄ are nucleotides. In preferred embodiments at least one of X₃ and X₄ is a G. In other embodiments both of X₃ and X₄ are a G. In yet other embodiments the preferred formula is 5′-GGGNGGG-3′ or 5′-GGGNGGGNGGG-3′, wherein N represents between 0 and 20 nucleotides. In other embodiments the poly-G nucleic acid is free of unmethylated CG dinucleotides, while in other embodiments the poly-G nucleic acid includes at least one unmethylated CG dinucleotide.

The poly-G nucleic acid in some embodiments is selected from the group consisting of SEQ ID NO: 5, 6, 67, 208, 258-260, 266, 272, 278, 287-289, 344, 348, 374, 375, 424, 454, 508, 534-536, 706, 741, 768, 769, 883-894, 897-900, 902-905, and 938. In other embodiments, the poly-G nucleic acid includes a sequence selected from the group consisting of SEQ ID NO: 61, 74-76, 134, 140, 141, 166, 171, 176, 178, 207, 216, 255, 256, 305, 318, 416, 417, 453, 496, 498-501, 503, 504, 512, 531, 542, 586, 604, 633-635, 698, 740, 796, 827, 828, 847, 863, 878, 895, 896, 901, and 906.

In certain embodiments, poly-G nucleic acids have the following formula:

5′-X₁X₂GGGX₃X₄-3′

wherein each of X₁, X₂, X₃, and X₄ is any nucleotide other than G. Such poly-G nucleic acids preferably do not include any of the formulas 5′-GXGGG-3′,5′-XGGGG-3′, or 5′-GGGXG-3′, wherein X is any nucleotide. Exemplary poly-G nucleic acids according to this embodiment include SEQ ID NO: 29, 48, 50, 122, 127, 136, 139, 192, 241, 264, 301, 356, 388, 446, 469, 482, 490, 547, 548, 823, 824, 986, and 1008.

Nucleic acids having modified backbones, such as phosphorothioate backbones, fall within the class of immunostimulatory nucleic acids. U.S. Pat. Nos. 5,723,335 and 5,663,153 issued to Hutcherson et al. and related PCT publication WO 95/26204 describe immune stimulation using phosphorothioate oligonucleotide analogues. These patents describe the ability of the phosphorothioate backbone to stimulate an immune response in a non-sequence specific manner.

For facilitating uptake into cells, the immunostimulatory nucleic acids are preferably in the range of 6 to 100 bases in length. However, nucleic acids of any size greater than 6 nucleotides (even many kb long) are capable of inducing an immune response according to the invention if sufficient immunostimulatory motifs are present. Preferably the immunostimulatory nucleic acid is in the range of between 8 and 100 and in some embodiments between 8 and 50, between 8 and 40, or between 8 and 30 nucleotides in length.

In the case when the immunostimulatory nucleic acid is administered in conjunction with a nucleic acid vector, it is preferred that the backbone of the immunostimulatory nucleic acid be a chimeric combination of phosphodiester and phosphorothioate (or other phosphate modification). The cell may have a problem taking up a plasmid vector in the presence of completely phosphorothioate oligonucleotide. Thus when both a vector and an oligonucleotide are delivered to a subject, it is preferred that the oligonucleotide have a chimeric backbone or have a phosphorothioate backbone but that the plasmid is associated with a vehicle that delivers it directly into the cell, thus avoiding the need for cellular uptake. Such vehicles are known in the art and include, for example, liposomes and gene guns.

For use in the instant invention, the immunostimulatory nucleic acids can be synthesized de novo using any of a number of procedures well known in the art. Such compounds are referred to as “synthetic nucleic acids.” For example, the β-cyanoethyl phosphoramidite method (Beaucage S L and Caruthers M H (1981) Tetrahedron Lett 22:1859-62); nucleoside H-phosphonate method (Garegg P J et al. (1986) Tetrahedron Lett 27:4051-4; Froehler B C et al. (1986) Nucl Acid Res 14:5399-407; Garegg P J et al. (1986) Tetrahedron Lett 27:4055-8; Gaffney B L et al. (1988) Tetrahedron Lett 29:2619-22). These chemistries can be performed by a variety of automated oligonucleotide synthesizers available in the market. These nucleic acids are referred to as synthetic nucleic acids. Alternatively, immunostimulatory nucleic acids can be produced on a large scale in plasmids, (see Sambrook, T., et al., “Molecular Cloning: A Laboratory Manual”, Cold Spring Harbor Laboratory Press, New York, 1989) and separated into smaller pieces or administered whole. Nucleic acids can be prepared from existing nucleic acid sequences (e.g., genomic or cDNA) using known techniques, such as those employing restriction enzymes, exonucleases or endonucleases. Nucleic acids prepared in this manner are referred to as isolated nucleic acids. The term “immunostimulatory nucleic acid” encompasses both synthetic and isolated immunostimulatory nucleic acids.

For use in vivo, nucleic acids are preferably relatively resistant to degradation (e.g., are stabilized). A “stabilized nucleic acid molecule” shall mean a nucleic acid molecule that is relatively resistant to in vivo degradation (e.g., via an exo- or endo-nuclease). Stabilization can be a function of length or secondary structure. Immunostimulatory nucleic acids that are tens to hundreds of kbs long are relatively resistant to in vivo degradation. For shorter immunostimulatory nucleic acids, secondary structure can stabilize and increase their effect. For example, if the 3′ end of a nucleic acid has self-complementarity to an upstream region, so that it can fold back and form a sort of stem loop structure, then the nucleic acid becomes stabilized and therefore exhibits more activity.

Alternatively, nucleic acid stabilization can be accomplished via backbone modifications. Preferred stabilized nucleic acids of the instant invention have a modified backbone. It has been demonstrated that modification of the nucleic acid backbone provides enhanced activity of the immunostimulatory nucleic acids when administered in vivo. One type of modified backbone is a phosphate backbone modification. Immunostimulatory nucleic acids, including at least two phosphorothioate linkages at the 5′ end of the oligonucleotide and multiple phosphorothioate linkages at the 3′ end, preferably 5, can in some circumstances provide maximal activity and protect the nucleic acid from degradation by intracellular exo- and endo-nucleases. Other phosphate modified nucleic acids include phosphodiester modified nucleic acids, combinations of phosphodiester and phosphorothioate nucleic acids, methylphosphonate, methylphosphorothioate, phosphorodithioate, p-ethoxy, and combinations thereof. Each of these combinations in CpG nucleic acids and their particular effects on immune cells is discussed in more detail in PCT published patent applications WO 96/02555 and WO 98/18810, the entire contents of which are hereby incorporated by reference. Another type of phosphate backbone modification is a p-ethoxy backbone modification as disclosed in U.S. Pat. No. 6,015,886. Although not meaning to be bound by the theory, it is believed that these phosphate modified nucleic acids may show more stimulatory activity due to enhanced nuclease resistance, increased cellular uptake, increased protein binding, and/or altered intracellular localization.

Modified backbones such as phosphorothioates may be synthesized using automated techniques employing either phosphoramidate or H-phosphonate chemistries. Aryl- and alkyl-phosphonates can be made, e.g., as described in U.S. Pat. No. 4,469,863, and alkylphosphotriesters (in which the charged oxygen moiety is alkylated as described in U.S. Pat. No. 5,023,243 and European Patent No. 092,574) can be prepared by automated solid phase synthesis using commercially available reagents. Methods for making other DNA backbone modifications and substitutions have been described (Uhlmann E and Peyman A (1990) Chem Rev 90:544-84; Goodchild J (1990) Bioconjugate Chem 1:165-87).

Both phosphorothioate and phosphodiester nucleic acids containing immunostimulatory motifs are active in immune cells. However, based on the concentration needed to induce immunostimulatory nucleic acid specific effects, the nuclease resistant phosphorothioate backbone immunostimulatory nucleic acids are more potent than unformulated phosphodiester (1 μg/ml for the phosphorothioate vs. >30 μg/ml for phosphodiester).

Another type of modified backbone, useful according to the invention, is a peptide nucleic acid. The backbone is composed of aminoethylglycine and supports bases which provide the DNA-like character. The backbone does not include any phosphate and thus may optionally have no net charge. The lack of charge allows for stronger DNA-DNA binding because the charge repulsion between the two strands does not exist. Additionally, because the backbone has an extra methylene group, the oligonucleotides are enzyme/protease resistant. Peptide nucleic acids can be purchased from various commercial sources, e.g., Perkin Elmer, Calif. or synthesized de novo.

Another class of backbone modifications include 2′-O-methylribonucleosides (2′-OMe). These types of substitutions are described extensively in the prior art and in particular with respect to their immunostimulating properties in Zhao Q et al. (1990) Bioorg Med Chem Lett 9:3453-8. Zhao et al. describes methods of preparing 2′-OMe modifications to nucleic acids.

The nucleic acid molecules of the invention may include naturally occurring or synthetic purine or pyrimidine heterocyclic bases as well as modified backbones. Purine or pyrimidine heterocyclic bases include, but are not limited to, adenine, guanine, cytosine, thymine, uracil, and inosine. Other representative heterocyclic bases are disclosed in U.S. Pat. No. 3,687,808, issued to Merigan, et al. The terms purine or pyrimidine or bases are used herein to refer to both naturally occurring or synthetic purines, pyrimidines or bases, and to analogs and derivatives thereof.

Other stabilized nucleic acids include: nonionic DNA analogs, such as alkyl- and aryl-phosphates (in which the charged phosphonate oxygen is replaced by an alkyl or aryl group), alkylphosphodiesters and alkylphosphotriesters, in which the charged oxygen moiety is alkylated. Nucleic acids which contain diol, such as tetraethyleneglycol or hexaethyleneglycol, at either or both termini have also been shown to be substantially resistant to nuclease degradation.

The immunostimulatory nucleic acids having backbone modifications useful according to the invention in some embodiments are S- or R-chiral immunostimulatory nucleic acids. An “S-chiral immunostimulatory nucleic acid” as used herein is an immunostimulatory nucleic acid wherein at least two nucleotides have a backbone modification forming a chiral center and wherein a plurality of the chiral centers have S chirality. An “R-chiral immunostimulatory nucleic acid” as used herein is an immunostimulatory nucleic acid wherein at least two nucleotides have a backbone modification forming a chiral center and wherein a plurality of the chiral centers have R chirality. The backbone modification may be any type of modification that forms a chiral center. The modifications include but are not limited to phosphorothioate, methylphosphonate, methylphosphorothioate, phosphorodithioate, 2′-OMe, and combinations thereof. To the extent that an R-chiral immunostimulatory nucleic acid may be degraded by cells or by the body more rapidly than its corresponding S-chiral immunostimulatory nucleic acid, the S-chiral immunostimulatory nucleic acid may be preferred over the R-chiral immunostimulatory nucleic acid.

The chiral immunostimulatory nucleic acids must have at least two nucleotides within the nucleic acid that have a backbone modification. All or less than all of the nucleotides in the nucleic acid, however, may have a modified backbone. Of the nucleotides having a modified backbone (referred to as chiral centers), a plurality have a single chirality, S or R. A “plurality” as used herein within the context of modified backbones refers to an amount greater than 75%. Thus, less than all of the chiral centers may have S- or R-chirality as long as a plurality of the chiral centers have S- or R-chirality. In some embodiments at least 80,%, 85%, 90%, 95%, or 100% of the chiral centers have S- or R-chirality. In other embodiments at least 80%, 85%, 90%, 95%, or 100% of the nucleotides have backbone modifications.

The S- and R-chiral immunostimulatory nucleic acids may be prepared by any method known in the art for producing chirally pure oligonucleotides. The reference by Stec et al. teaches methods for producing stereopure phosphorothioate oligodeoxynucleotides using an oxathiaphospholane. Stec W J et al. (1995) J Am Chem Soc 117:12019. Other methods for making chirally pure oligonucleotides have been described by companies such as ISIS Pharmaceuticals. U.S. patents have also described these methods. For instance U.S. Pat. Nos. 5,883,237; 5,837,856; 5,599,797; 5,512,668; 5,856,465; 5,359,052; 5,506,212; 5,521,302; and 5,212,295, each of which is hereby incorporated by reference in its entirety, disclose methods for generating stereopure oligonucleotides.

The immunostimulatory nucleic acids are useful for treating or preventing non-allergic inflammatory disease in a subject. A “subject” shall mean a human or vertebrate mammal including but not limited to a dog, cat, horse, cow, pig, sheep, goat, or primate, e.g., monkey.

As used herein, the terms “prevent,” “prevented,” or “preventing” when used with respect to the treatment of a particular disorder refers to a prophylactic treatment of a subject which increases the resistance of the subject to development or exacerbation of the particular disorder. In other words, such prophylactic treatment decreases the likelihood that the treated subject will develop a particular disorder or experience an exacerbation of a previously established disorder. Prophylactic or preventive treatment as used herein thus can reduce or eliminate a disorder altogether or prevent it from becoming worse. Accordingly, the terms “prevent,” “prevented,” or “preventing” when used with respect to the treatment of a non-allergic inflammatory disease refers to a prophylactic treatment of a subject which increases the resistance of the subject to development or exacerbation of the non-allergic inflammatory disease.

The immunostimulatory nucleic acids may also be delivered to the subject in the form of a plasmid vector. In some embodiments, one plasmid vector could include both the immunostimulatory nucleic acid and a nucleic acid encoding a protein non-allergic inflammatory disease medicament. In other embodiments, separate plasmids could be used. In yet other embodiments, no plasmids could be used.

The compositions of the invention may be delivered to the immune system or other target cells alone or in association with a vector. In its broadest sense, a “vector” is any vehicle capable of facilitating the transfer of the compositions to the target cells. The vector generally transports the nucleic acid to the immune cells with reduced degradation relative to the extent of degradation that would result in the absence of the vector.

In general, the vectors useful in the invention are divided into two classes: biological vectors and chemical/physical vectors. Biological vectors and chemical/physical vectors are useful for delivery/uptake of nucleic acids, non-allergic inflammatory disease medicaments, and/or other active agents to/by a target cell.

Biological vectors include, but are not limited to, plasmids, phagemids, viruses, other vehicles derived from viral or bacterial sources that have been manipulated by the insertion or incorporation of nucleic acid sequences, and free nucleic acid fragments which can be attached to nucleic acid sequences. Viral vectors are a preferred type of biological vector and include, but are not limited to, nucleic acid sequences from the following viruses: retroviruses, such as: HIV-1, HIV-2, HTLV-I, HTLV-II, Moloney murine leukemia virus, Harvey murine sarcoma virus, murine mammary tumor virus, Rous sarcoma virus; adenovirus; adeno-associated virus; SV40-type viruses; polyoma viruses; Epstein-Barr viruses; papilloma viruses; herpes viruses; vaccinia viruses; polio viruses; and RNA viruses. One can readily employ other viral vectors not named but known in the art.

Preferred viral vectors are based on non-cytopathic eukaryotic viruses in which non-essential genes have been replaced with a nucleic acid of interest. Non-cytopathic viruses include retroviruses, the life cycle of which involves reverse transcription of genomic viral RNA into DNA with subsequent proviral integration into host cellular DNA. Retroviruses have been approved for human gene therapy trials. In general, the retroviruses are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Kriegler, M., “Gene Transfer and Expression, A Laboratory Manual,” W.H. Freeman Co., New York (1990) and Murry, E. J. Ed. “Methods in Molecular Biology,” vol. 7, Humana Press, Inc., Cliffton, N.J. (1991).

Another preferred virus for certain applications is the adeno-associated virus, a double-stranded DNA virus. The adeno-associated virus (AAV) can be engineered to be replication-deficient and is capable of infecting a wide range of cell types and species. It further has advantages, such as heat and lipid solvent stability; high transduction frequencies in cells of diverse lineages; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression. In addition, wild-type adeno-associated virus infections have been followed in tissue culture for greater than 100 passages in the absence of selective pressure, implying that the AAV genomic integration is a relatively stable event. The adeno-associated virus can also function in an extrachromosomal fashion.

Other biological vectors include plasmid vectors. Plasmid vectors have been extensively described in the art and are well known to those of skill in the art. See e.g., Sambrook et al., “Molecular Cloning: A Laboratory Manual,” Second Edition, Cold Spring Harbor Laboratory Press, 1989. In the last few years, plasmid vectors have been found to be particularly advantageous for delivering genes to cells in vivo because of their inability to replicate within and integrate into a host genome. These plasmids, however, having a promoter compatible with the host cell, can express a peptide from a gene operatively encoded within the plasmid. Some commonly used plasmids include pBR322, pUC18, pUC19, pRc/CMV, SV40, and pBlueScript. Other plasmids are well known to those of ordinary skill in the art. Additionally, plasmids may be custom designed using restriction enzymes and ligation reactions to remove and add specific fragments of DNA.

Gene-carrying plasmids can be delivered to the immune system using bacteria. Modified forms of bacteria such as Salmonella can be transfected with the plasmid and used as delivery vehicles. The bacterial delivery vehicles can be administered to a host subject orally or by other administration means. The bacteria deliver the plasmid to immune cells, e.g., B cells and dendritic cells, likely by passing through the gut barrier. High levels of immune protection have been established using this methodology. Such methods of delivery are useful for the aspects of the invention utilizing systemic delivery of immunostimulatory nucleic acid and/or other therapeutic agent.

In addition to the biological vectors, chemical/physical vectors may be used to deliver a nucleic acid, non-allergic inflammatory disease medicament, and/or other therapeutic agent to a target cell and facilitate uptake thereby. As used herein, a “chemical/physical vector” refers to a natural or synthetic molecule, other than those derived from bacteriological or viral sources, capable of delivering the nucleic acid, non-allergic inflammatory disease medicament, and/or other therapeutic agent to a cell.

A preferred chemical/physical vector of the invention is a colloidal dispersion system. Colloidal dispersion systems include lipid-based systems including oil-in-water emulsions, water-in-oil emulsions, micelles, mixed micelles, and liposomes. A preferred colloidal system of the invention is a liposome. Liposomes are artificial membrane vessels which are useful as a delivery vector in vivo or in vitro. It has been shown that large unilamellar vesicles (LUV), which range in size from 0.2 μm -4.0 μm can encapsulate large macromolecules. RNA, DNA, and intact virions can be encapsulated within the aqueous interior and be delivered to cells in a biologically active form. Fraley et al. (1981) Trends Biochem Sci 6:77.

Liposomes may be targeted to a particular tissue by coupling the liposome to a specific ligand such as a monoclonal antibody, sugar, glycolipid, or protein. Ligands which may be useful for targeting a liposome to an immune cell include, but are not limited to: intact molecules or fragments of molecules which interact with immune cell-specific receptors and molecules, such as antibodies, which interact with the cell surface markers of immune cells. Such ligands may easily be identified by binding assays well known to those of skill in the art. Additionally, the vector may be coupled to a nuclear targeting peptide, which will direct the vector to the nucleus of the host cell.

Lipid formulations for transfection are commercially available from QIAGEN, for example, as EFFECTENE™ (a non-liposomal lipid with a special DNA condensing enhancer) and SUPERFECT™ (a novel acting dendrimeric technology).

Liposomes are commercially available from Gibco BRL, for example, as LIPOFECTIN™ and LIPOFECTACE™, which are formed of cationic lipids such as N-[1-(2, 3 dioleyloxy)-propyl]-N, N, N-trimethylammonium chloride (DOTMA) and dimethyl dioctadecylammonium bromide (DDAB). Methods for making liposomes are well known in the art and have been described in many publications. Liposomes also have been reviewed by Gregoriadis G (1985) Trends Biotechnol 3:235-241.

In one embodiment, the vehicle is a biocompatible microparticle or implant that is suitable for implantation or administration to the mammalian recipient. Exemplary bioerodable implants that are useful in accordance with this method are described in PCT International application no. PCT/US95/03307 (Publication No. WO 95/24929, entitled “Polymeric Gene Delivery System”). PCT/US/03307 describes a biocompatible, preferably biodegradable polymeric matrix for containing an exogenous gene under the control of an appropriate promoter. An exemplary biocompatible, biodegradable polymeric matrix is poly(lactide-glycolide) (PLGA). The polymeric matrix can be used to achieve sustained release of the exogenous gene in the patient.

The polymeric matrix preferably is in the form of a microparticle such as a microsphere (wherein the nucleic acid, non-allergic inflammatory disease medicament, and/or other pharmaceutical agent is dispersed throughout a solid polymeric matrix) or a microcapsule (wherein the nucleic acid, non-allergic inflammatory disease medicament, and/or other pharmaceutical agent is stored in the core of a polymeric shell). Other forms of the polymeric matrix for containing the nucleic acid, non-allergic inflammatory disease medicament, and/or other pharmaceutical agent include films, coatings, gels, implants, and stents. The size and composition of the polymeric matrix device is selected to result in favorable release kinetics in the tissue into which the matrix is introduced. The size of the polymeric matrix further is selected according to the method of delivery which is to be used, typically injection into a tissue or administration of a suspension by aerosol into the nasal and/or pulmonary areas. Preferably when an aerosol route is used the polymeric matrix and the nucleic acid, non-allergic inflammatory disease medicament, and/or other pharmaceutical agent are encompassed in a surfactant vehicle. The polymeric matrix composition can be selected to have both favorable degradation rates and also to be formed of a material which is bioadhesive, to further increase the effectiveness of transfer when the matrix is administered to a nasal and/or pulmonary surface that has sustained an injury. The matrix composition also can be selected not to degrade, but rather, to release by diffusion over an extended period of time.

In another embodiment the chemical/physical vector is a biocompatible microsphere that is suitable for delivery, such as oral or mucosal delivery. Such microspheres are disclosed in Chickering et al. (1996) Biotechnol Bioeng 52:96-101, Mathiowitz E et al. (1997) Nature 386:410-4, and PCT published patent application WO 97/03702.

Both non-biodegradable and biodegradable polymeric matrices can be used to deliver the nucleic acid, non-allergic inflammatory disease medicament, and/or other pharmaceutical agent to the subject. Biodegradable matrices are preferred. Such polymers may be natural or synthetic polymers. The polymer is selected based on the period of time over which release is desired, generally in the order of a few hours to a year or longer. Typically, release over a period ranging from between a few hours and three to twelve months is most desirable. The polymer optionally is in the form of a hydrogel that can absorb up to about 90% of its weight in water and further, optionally is cross-linked with multi-valent ions or other polymers.

Bioadhesive polymers of particular interest include bioerodible hydrogels described by Sawhney H S et al. (1993) Macromolecules 26:581-587, the teachings of which are incorporated herein, polyhyaluronic acids, casein, gelatin, gluten, polyanhydrides, polyacrylic acid, alginate, chitosan, poly(methyl methacrylates), poly(ethyl methacrylates), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

Compaction agents also can be used alone, or in combination with, a biological or chemical/physical vector. A “compaction agent,” as used herein, refers to an agent, such as a histone, that neutralizes the negative charges on the nucleic acid and thereby permits compaction of the nucleic acid into a fine granule. Compaction of the nucleic acid facilitates the uptake of the nucleic acid by the target cell. The compaction agents can be used alone, i.e., to deliver a nucleic acid in a form that is more efficiently taken up by the cell or, more preferably, in combination with one or more of the above-described vectors. In some embodiments the compaction agent may be PLGA.

Other exemplary compositions that can be used to facilitate uptake by a target cell of the nucleic acid, non-allergic inflammatory disease medicament, and/or other pharmaceutical agent include calcium phosphate and other chemical mediators of intracellular transport, microinjection compositions, electroporation, and homologous recombination compositions (e.g., for integrating a nucleic acid into a preselected location within the target cell chromosome).

The immunostimulatory nucleic acid and/or the non-allergic inflammatory disease medicament and/or other therapeutic agents may be administered alone (e.g., in saline or buffer) or using any delivery vectors known in the art. For instance the following delivery vehicles have been described: Cochleates (Gould-Fogerite et al., 1994, 1996); Emulsomes (Vancott et al., 1998, Lowell et al., 1997); ISCOMs (Mowat et al., 1993, Carlsson et al., 1991, Hu et., 1998, Morein et al., 1999); Liposomes (Childers et al., 1999, Michalek et al., 1989, 1992, de Haan 1995a, 1995b); Live bacterial vectors (e.g., Salmonella, Escherichia coli, Bacillus Calmette-Guerin (BCG), Shigella, Lactobacillus) (Hone et al., 1996, Pouwels et al., 1998, Chatfield et al., 1993, Stover et al., 1991, Nugent et al., 1998); Live viral vectors (e.g., Vaccinia, adenovirus, Herpes Simplex) (Gallichan et al., 1993, 1995, Moss et al., 1996, Nugent et al., 1998, Flexner et al., 1988, Morrow et al., 1999); Microspheres (Gupta et al., 1998, Jones et al., 1996, Maloy et al., 1994, Moore et al., 1995, O'Hagan et al., 1994, Eldridge et al., 1989); Nucleic acid vaccines (Fynan et al., 1993, Kuklin et al., 1997, Sasaki et al., 1998, Okada et al., 1997, Ishii et al., 1997); Polymers (e.g. carboxymethylcellulose, chitosan) (Hamajima et al., 1998, Jabbal-Gill et al., 1998); Polymer rings (Wyatt et al., 1998); Proteosomes (Vancott et al., 1998, Lowell et al., 1988, 1996, 1997); Sodium Fluoride (Hashi et al., 1998); Transgenic plants (Tacket et al., 1998, Mason et al., 1998, Haq et al., 1995); Virosomes (Gluck et al., 1992, Mengiardi et al., 1995, Cryz et al., 1998); Virus-like particles (Jiang et al., 1999, Leibl et al., 1998).

The immunostimulatory nucleic acid and non-allergic inflammatory disease medicament can be combined with other therapeutic agents such as adjuvants to enhance immune responses even further. The immunostimulatory nucleic acid, non-allergic inflammatory disease medicament and other therapeutic agent may be administered simultaneously or sequentially. When the immunostimulatory nucleic acid, non-allergic inflammatory disease medicament or other therapeutic agents are administered simultaneously, they can be administered in the same or separate formulations, but are administered at the same time. The other therapeutic agents are administered sequentially with one another and with the immunostimulatory nucleic acid and non-allergic inflammatory disease medicament, when the administration of the other therapeutic agents and the immunostimulatory nucleic acid and non-allergic inflammatory disease medicament is temporally separated. The separation in time between the administration of these compounds may be a matter of minutes or it may be longer. Other therapeutic agents include but are not limited to non-nucleic acid adjuvants, cytokines, antibodies, antigens, etc.

A “non-nucleic acid adjuvant” is any molecule or compound except for the immunostimulatory nucleic acids described herein which can stimulate the humoral and/or cellular immune response. Non-nucleic acid adjuvants include, for instance, adjuvants that create a depot effect, immune stimulating adjuvants, adjuvants that both create a depot effect and stimulate the immune system, and non-nucleic acid mucosal adjuvants.

An “adjuvant that creates a depot effect” as used herein is an adjuvant that causes an antigen or allergen to be slowly released in the body, thus prolonging the exposure of immune cells to the antigen or allergen. This class of adjuvants includes but is not limited to alum (e.g., aluminum hydroxide, aluminum phosphate); or emulsion-based formulations including mineral oil, non-mineral oil, water-in-oil or oil-in-water-in-oil emulsion, oil-in-water emulsions such as Seppic ISA series of Montanide adjuvants (e.g., Montanide ISA 720, AirLiquide, Paris, France); MF-59 (a squalene-in-water emulsion stabilized with Span 85 and Tween 80; Chiron Corporation, Emeryville, Calif.); and PROVAX (an oil-in-water emulsion containing a stabilizing detergent and a micelle-forming agent; IDEC Pharmaceuticals Corporation, San Diego, Calif.).

An “immune stimulating adjuvant” is an adjuvant that causes activation of a cell of the immune system. It may, for instance, cause an immune cell to produce and secrete cytokines. This class of adjuvants includes but is not limited to saponins purified from the bark of the Q. saponaria tree, such as QS21 (a glycolipid that elutes in the 21^{st} peak with HPLC fractionation; Aquila Biopharmaceuticals, Inc., Worcester, Mass.); poly[di(carboxylatophenoxy)phosphazene] (PCPP polymer; Virus Research Institute, USA); derivatives of lipopolysaccharides such as monophosphoryl lipid A (MPL; Ribi ImmunoChem Research, Inc., Hamilton, Mont.), muramyl dipeptide (MDP; Ribi) and threonyl-muramyl dipeptide (t-MDP; Ribi); OM-1 74 (a glucosamine disaccharide related to lipid A; OM Pharma SA, Meyrin, Switzerland); and Leishmania elongation factor (a purified Leishmania protein; Corixa Corporation, Seattle, Wash.).

An “adjuvant that both creates a depot effect and stimulates the immune system” is an adjuvant that has both of the above-identified functions. This class of adjuvants includes but is not limited to ISCOMS (Immunostimulating complexes which contain mixed saponins, lipids and form virus-sized particles with pores that can hold antigen; CSL, Melbourne, Australia); SB-AS2 (SmithKline Beecham adjuvant system #2 which is an oil-in-water emulsion containing MPL and QS21; SmithKline Beecham Biologicals [SBB], Rixensart, Belgium); SB-AS4 (SmithKline Beecham adjuvant system #4 which contains alum and MPL; SBB, Belgium); non-ionic block copolymers that form micelles such as CRL 1005 (these contain a linear chain of hydrophobic polyoxpropylene flanked by chains of polyoxyethylene; Vaxcel, Inc., Norcross, Ga.); and Syntex Adjuvant Formulation (SAF, an oil-in-water emulsion containing Tween 80 and a nonionic block copolymer; Syntex Chemicals, Inc., Boulder, Colo.).

A “non-nucleic acid mucosal adjuvant” as used herein is an adjuvant other than an immunostimulatory nucleic acid that is capable of inducing a mucosal immune response in a subject when administered to a mucosal surface in conjunction with an antigen or allergen. Mucosal adjuvants include but are not limited to Bacterial toxins: e.g., Cholera toxin (CT), CT derivatives including but not limited to CT B subunit (CTB) (Wu et al., 1998, Tochikubo et al., 1998); CTD53 (Val to Asp) (Fontana et al., 1995); CTK97 (Val to Lys) (Fontana et al., 1995); CTK104 (Tyr to Lys) (Fontana et al., 1995); CTD53/K63 (Val to Asp, Ser to Lys) (Fontana et al., 1995); CTH54 (Arg to His) (Fontana et al., 1995); CTN107 (His to Asn) (Fontana et al., 1995); CTE1 14 (Ser to Glu) (Fontana et al., 1995); CTE112K (Glu to Lys) (Yamamoto et al., 1997a); CTS61F (Ser to Phe) (Yamamoto et al., 1997a, 1997b); CTS106 (Pro to Lys) (Douce et al., 1997, Fontana et al., 1995); and CTK63 (Ser to Lys) (Douce et al., 1997, Fontana et al., 1995), Zonula occludens toxin, zot, Escherichia coli heat-labile enterotoxin, Labile Toxin (LT), LT derivatives including but not limited to LT B subunit (LTB) (Verweij et al., 1998); LT7K (Arg to Lys) (Komase et al., 1998, Douce et al., 1995); LT61F (Ser to Phe) (Komase et al., 1998); LT1 12K (Glu to Lys) (Komase et al., 1998); LT118E (Gly to Glu) (Komase et al., 1998); LT146E (Arg to Glu) (Komase et al., 1998); LT192G (Arg to Gly) (Komase et al., 1998); LTK63 (Ser to Lys) (Marchetti et al., 1998, Douce et al., 1997, 1998, Di Tommaso et al., 1996); and LTR72 (Ala to Arg) (Giuliani et al., 1998), Pertussis toxin, PT. (Lycke et al., 1992, Spangler B D, 1992, Freytag and Clemments, 1999, Roberts et al., 1995, Wilson et al., 1995) including PT-9K/129G (Roberts et al., 1995, Cropley et al., 1995); Toxin derivatives (see below) (Holmgren et al., 1993, Verweij et al., 1998, Rappuoli et al., 1995, Freytag and Clements, 1999); Lipid A derivatives (e.g., monophosphoryl lipid A, MPL) (Sasaki et al., 1998, Vancott et al., 1998; Muramyl Dipeptide (MDP) derivatives (Fukushima et al., 1996, Ogawa et al., 1989, Michalek et al., 1983, Morisaki et al., 1983); Bacterial outer membrane proteins (e.g., outer surface protein A (OspA) lipoprotein of Borrelia burgdorferi; outer membrane protein of Neisseria meningitidis) (Marinaro et al., 1999, Van de Verg et al., 1996); Oil-in-water emulsions (e.g., MF59) (Barchfield et al., 1999, Verschoor et al., 1999, O'Hagan, 1998); Aluminum salts (Isaka et al., 1998, 1999); and Saponins (e.g., QS21) Aquila Biopharmaceuticals, Inc., Worcester, Mass.) (Sasaki et al., 1998, MacNeal et al., 1998), ISCOMS, MF-59 (a squalene-in-water emulsion stabilized with Span 85 and Tween 80; Chiron Corporation, Emeryville, Calif.); the Seppic ISA series of Montanide adjuvants (e.g., Montanide ISA 720; AirLiquide, Paris, France); PROVAX (an oil-in-water emulsion containing a stabilizing detergent and a micell-forming agent; IDEC Pharmaceuticals Corporation, San Diego, Calif.); Syntext Adjuvant Formulation (SAF; Syntex Chemicals, Inc., Boulder, Colo.); poly[di(carboxylatophenoxy)phosphazene (PCPP polymer; Virus Research Institute, USA) and Leishmania elongation factor (Corixa Corporation, Seattle, Wash.).

Immune responses can also be induced or augmented by the co-administration or co-linear expression of cytokines (Bueler et al., 1996; Chow et al., 1997; Geissler et al., 1997; Iwasaki et al., 1997; Kim et al., 1997) or B7 co-stimulatory molecules (Iwasaki et al., 1997; Tsuji et al., 1997) with the immunostimulatory nucleic acids. The cytokines can be administered directly with immunostimulatory nucleic acids or may be administered in the form of a nucleic acid vector that encodes the cytokine, such that the cytokine can be expressed in vivo. In one embodiment, the cytokine is administered in the form of a plasmid expression vector. In one embodiment, the cytokine is a Th1 cytokine. In one embodiment the cytokine is a Th2 cytokine.

The term “cytokine” is used as a generic name for a diverse group of soluble proteins and peptides which act as humoral regulators at nanomolar to picomolar concentrations and which, either under normal or pathological conditions, modulate the functional activities of individual cells and tissues. These proteins also mediate interactions between cells directly and regulate processes taking place in the extracellular environment. Examples of cytokines include, but are not limited to interleukin (IL)-1, IL-2, IL-4, IL-5, IL-6, IL-7, IL-10, IL-12, IL-13, IL-15, IL-18, granulocyte-macrophage colony stimulating factor (GM-CSF), granulocyte colony stimulating factor (G-CSF), interferon-γ (IFN-γ), IFN-α, tumor necrosis factor (TNF), transforming growth factor (TGF)-β, FLT-3 ligand, and CD40 ligand. Th1 cytokines include, without limitation, IFN-γ, IL-2, IL-12, IL-18, and TNF; Th2 cytokines include, without limitation, IL-4, IL-5, IL-10, and IL-13. Cytokines play a role in directing the T cell response. Helper (CD4+) T cells orchestrate the immune response of mammals through production of soluble factors that act on other immune system cells, including other T cells. Most mature CD4+T helper cells express one of two cytokine profiles: Th1 or Th2. In some embodiments it is preferred that the cytokine be a Th1 cytokine.

The term “effective amount” as used with reference to a medicament or therapeutic agent refers to an amount necessary or sufficient to realize a desired biologic effect. Thus an effective amount of an immunostimulatory nucleic acid refers to an amount of an immunostimulatory nucleic acid necessary or sufficient to realize a desired biologic effect. For example, an effective amount of an immunostimulatory nucleic acid for treating or preventing a non-allergic inflammatory disease is that amount necessary to reduce clinical manifestations or prevent development of a non-allergic inflammatory disease. That is, an effective amount of an immunostimulatory nucleic acid for treating or preventing a non-allergic inflammatory disease in a subject is that amount necessary to reduce or prevent non-allergic inflammation in a tissue of the subject. The effectuation of the reducing or preventing can be assessed by applying clinical, histologic, or other laboratory standards recognized by those skilled in the art as appropriate for diagnosing or assessing the non-allergic inflammatory disease being treated.

For example, the effectiveness of treatment for psoriasis can be determined by serial clinical examination of the skin, with or without determination of a psoriasis area and severity index (PASI) score. The PASI takes account of the extent of affected skin and the intensity of the three main lesions: erythema, desquamation, and infiltration. Fredriksson T et al. (1978) Dermatologica 157:238-44. The extent of lesions is from none (0) to 90-100% of skin. Each type of lesion is scored as absent (0), slight (1), moderate (2), marked (3), or severe (4). Evaluations are made for four body regions: head (0.1 of skin surface area), trunk (0.3 of skin surface area), upper limbs (0.2 of skin surface area), and lower limbs (0.4 of skin surface area). The higher the index, the more severe the disease. A PASI of 12 or more is generally considered to be indicative of moderate to severe psoriasis.

The effectiveness of treatment for psoriasis can also be measured, without limitation, according to any of the following measures: (1) erythematous skin with loose whitish scales; (2) acanthosis, hyperkeratosis and focal parakeratosis; (3) keratinocyte hyperproliferation; (4) changes in keratinocyte differentiation; (5) increased expression of MHC class II in one or more skin lesions; (6) increased expression of ICAM-1 in one or more skin lesions; (7) dermal angiogenesis; (8) dilation of blood vessels; (9) increased number of dermal mast cells; (10) infiltration of the dermis with neutrophils; (11) formation of microabscesses within the epidermis; and (12) changes in cytokine expression patterns that exhibit changes in cytokine expression patterns observed in the skin of human patients with psoriasis.

Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial toxicity and yet is effective to treat the particular subject. The effective amount for any particular application can vary depending on such factors as the disease or condition being treated, the severity of the disease or condition, the size of the subject, and the particular immunostimulatory nucleic acid and/or non-allergic inflammatory disease medicament being administered (e.g., for a CpG nucleic acid, the effective amount may vary depending on the number of unmethylated CpG motifs, their location in the nucleic acid, and the degree of modification of the backbone of the oligonucleotide). One of ordinary skill in the art can empirically determine the effective amount of a particular immunostimulatory nucleic acid and/or non-allergic inflammatory disease medicament and/or other therapeutic agent without necessitating undue experimentation.

Subject doses of the compounds described herein typically range from about 0.1 μg to 10,000 mg, more typically from about 1 μg to 8000 mg, and most typically from about 10 μg to 100 mg. Stated in terms of subject body weight, typical dosages range from about 0.1 μg/kg/day to 0.2 mg/kg/day, more typically from about 0.01 to 0.1 mg/kg/day, and most typically from about 0.01 to 0.05 mg/kg/day. In certain preferred embodiments, dosages range from 1 μg/kg/day to 1000 μg/kg/day, and more preferably from 10 μg/kg/week. to 100 μg/kg/week. In embodiments calling for systemic administration of immunostimulatory nucleic acid in the treatment of psoriasis, preferred doses of immunostimulatory nucleic acid range from 3 μg/kg/week to 30 μg/kg/week. Dosing in topical administration can vary with the efficiency of the particular formulation selected and the area of application; those of ordinary skill in the art will readily be able to select topical doses based on factors such as clinical response, side effects, and in vivo and in vitro assays of immune response.

In other embodiments of the invention, the immunostimulatory nucleic acid is administered on a routine schedule. The non-allergic inflammatory disease medicament may also be administered on a routine schedule, but alternatively, may be administered as symptoms arise. A “routine schedule” as used herein, refers to a predetermined designated period of time. The routine schedule may encompass periods of time which are identical or which differ in length, as long as the schedule is predetermined. For instance, the routine schedule may involve administration of the immunostimulatory nucleic acid every day, every two days, every three days, every four days, every five days, every six days, every week, every two weeks, every three weeks, every month, every two months, or any set number of days or weeks there-between, every three months, or every four months, five months, six months, seven months, eight months, nine months, ten months, eleven months, twelve months, etc. Alternatively, the predetermined routine schedule may involve administration of the immunostimulatory nucleic acid on a daily basis for the first week, followed by a monthly basis for several months, and then every three months after that. Any particular combination would be covered by the routine schedule as long as it is determined ahead of time that the appropriate schedule involves administration on a certain day.

According to certain embodiments where the non-allergic inflammatory disease involves an epithelium, the disease may be treated or prevented by administering the immunostimulatory nucleic acid locally to epithelium that is already affected or damaged by the non-allergic inflammatory disease. For example, in treating psoriasis according to certain embodiments of the invention, the immunostimulatory nucleic acid can be administered locally to areas of erythematous skin with loose whitish scales characteristic of psoriasis.

According to certain embodiments where the non-allergic inflammatory disease involves an epithelium, the disease may be treated or prevented by administering the immunostimulatory nucleic acid locally to intact epithelium, e.g., to epithelium that at the time of treatment is not clinically affected by the non-allergic inflammatory disease. For example, in treating psoriasis according to certain embodiments of the invention, the immunostimulatory nucleic acid can be administered locally to areas of skin not presently involved in the psoriasis, as evidenced by characteristic erythematous skin with loose whitish scales. For local administration involving the skin, the immunostimulatory nucleic acid can be administered by routes including topical, transdermal, subcutaneous. Similarly, with respect to inflammatory bowel disease, particularly Crohn's disease, according to certain embodiments of the invention the immunostimulatory nucleic acid can be administered locally to areas of intact mucosa lining, for example, the large or small bowel. For local administration involving intact mucosa lining the bowel, the immunostimulatory nucleic acid can be administered by routes including oral, rectal, retention enema, endoscopic, and the like.

In some aspects of the invention, the immunostimulatory nucleic acid is administered to the subject in anticipation of a disease flare triggering event in order to prevent a disease flare event. The anticipated disease flare triggering event may be, but need not be limited to, sun esposure, exposure to or withdrawal of certain drugs, infection, and psychological stress. In some instances, the immunostimulatory nucleic acid is administered substantially prior to a disease flare triggering event. As used herein, “substantially prior” means at least six months, at least five months, at least four months, at least three months, at least two months, at least one month, at least three weeks, at least two weeks, at least one week, at least 5 days, or at least 2 days prior to the disease flare triggering event.

Similarly, the non-allergic inflammatory disease medicament may be administered immediately prior to the disease flare trigging event (e.g., within 48 hours, within 24 hours, within 12 hours, within 6 hours, within 4 hours, within 3 hours, within 2 hours, within 1 hour, within 30 minutes or within 10 minutes of a disease flare triggering event), substantially simultaneously with the disease flare triggering event, or following the disease flare triggering event.

In some embodiments, the immunostimulatory nucleic acid and the non-allergic inflammatory disease medicament are both administered to a subject. The timing of administration of both may vary. In some embodiments, it is preferred that the non-allergic inflammatory disease medicament be administered subsequent to the administration of the immunostimulatory nucleic acid. In some embodiments, the immunostimulatory nucleic acid is administered to the subject prior to as well as either substantially simultaneously with or following the administration of the non-allergic inflammatory disease medicament. The administration of the immunostimulatory nucleic acid and the non-allergic inflammatory disease medicament may also be mutually exclusive of each other so that at any given time during the treatment period, only one of these agents is active in the subject. Alternatively, and preferably in some instances, the administration of the two agents overlaps such that both agents are active in the subject at the same time.

In other aspects, the invention relates to kits that are useful in the treatment of non-allergic inflammatory disease. One kit of the invention includes a sustained-release vehicle containing an immunostimulatory nucleic acid, a container housing a non-allergic inflammatory disease medicament, and instructions for timing of administration of the immunostimulatory nucleic acid and the non-allergic inflammatory disease medicament. A sustained-release vehicle is used herein in accordance with its prior art meaning of any device which slowly releases the immunostimulatory nucleic acid.

Such systems can avoid repeated administrations of the compounds, increasing convenience to the subject and the physician. Many types of release delivery systems are available and known to those of ordinary skill in the art. They include polymer-based systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono-, di-, and tri-glycerides; hydrogel release systems; silastic systems; peptide-based systems; wax coatings; compressed tablets using conventional binders and excipients; partially fused implants; and the like. Specific examples include, but are not limited to: (a) erosional systems in which an agent of the invention is contained in a form within a matrix such as those described in U.S. Pat. Nos. 4,452,775, 4,675,189, and 5,736,152, and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation.

The non-allergic inflammatory disease medicament is housed in at least one container. The container may be a single container housing all of the non-allergic inflammatory disease medicament together, or it may be multiple containers or chambers housing individual dosages of the non-allergic inflammatory disease medicament, such as a blister pack. The kit also has instructions for timing of administration of the non-allergic inflammatory disease medicament. The instructions would direct the subject having non-allergic inflammatory disease or at risk of non-allergic inflammatory disease to take the non-allergic inflammatory disease medicament at the appropriate time. For instance, the appropriate time for delivery of the medicament may be as the symptoms occur. Alternatively, the appropriate time for administration of the medicament may be on a routine schedule such as daily, weekly, monthly or yearly.

In other aspects of the invention, a composition is provided. The composition includes an immunostimulatory nucleic acid and a non-allergic inflammatory disease medicament formulated in a pharmaceutically acceptable carrier and present in the composition in an effective amount for preventing or treating an immune or inflammatory response associated with a non-allergic inflammatory disease. The effective amount for preventing or treating an immune or inflammatory response is that amount which prevents, inhibits completely, or inhibits partially the induction of the immune or inflammatory response, or which prevents an increase in the immune or inflammatory response associated with a non-allergic inflammatory disease. An immune or inflammatory response associated with a non-allergic inflammatory disease includes, for example, induced or increased expression of certain cytokines, such as IL-12, IFN-α and TNF-α, in association with the recruitment and localization of neutrophils, without concomitant significantly induced or increased expression of IgE. As previously described herein, the immune cells principally involved in inflammation include granulocytes (neutrophils, eosinosphils, and basophils), phagocytic cells (monocytes and macrophages), natural killer (NK) cells, and T lymphocytes (T cells). Monocytes and macrophages phagocytose materials foreign to the host and degrade them within lysosomes. These cells also secrete enzymes, reactive oxygen species, and lipid mediators including leukotrienes and prostaglandins, all of which can not only serve to protect the host but also can cause unwanted damage to uninvolved bystander cells. The inflammatory response further includes the recruitment and localization of neutrophils and other inflammatory cells, under the direction of cytokines and chemokines secreted by the monocytes and macrophages. Among the principal cytokines involved in inflammation are IFN-α, IFN-β, IFN-γ, TNF-α, TNF-β, IL-1β, IL-6, IL-8, and IL-12. Additional soluble factors released as part of the inflammatory response include certain plasma proteases, including complement; vasoactive kinins, including bradykinin; and clotting and fibrinolytic factors (factor XII and plasmin). Thus an immune or inflammatory response associated with a non-allergic inflammatory disease is meant to encompass any of these features, and typically some combination of such features.

In other aspects of the invention, a composition is provided that is suitable for topical administration to a subject. The composition includes an immunostimulatory nucleic acid formulated as a lotion, cream, ointment, gel, or transdermal patch in a pharmaceutically acceptable carrier and present in the composition in an effective amount for preventing or treating an immune or inflammatory response associated with a non-allergic inflammatory disease. In one embodiment the immunostimulatory nucleic acid is a CpG nucleic acid. In one embodiment the immunostimulatory nucleic acid is a methylated CpG nucleic acid. In one embodiment the immunostimulatory nucleic acid is a poly-G nucleic acid. In one embodiment the immunostimulatory nucleic acid is a T-rich nucleic acid. Lotions, creams, ointments, and gels can be formulated with an aqueous or oily base alone or together with suitable thickening and/or gelling agents. Lotions can be formulated with an aqueous or oily base and, typically, further include one or more emulsifying agents, stabilizing agents, dispersing agents, suspending agents, thickening agents, or coloring agents. (See, e.g., U.S. Pat. No. 5,563,153, entitled “Sterile Topical Anesthetic Gel”, issued to Mueller, D., et al., for a description of a pharmaceutically acceptable gel-based topical carrier.) The lotion, cream, ointment, or gel can optionally be formulated to include sunscreen compounds, fragrance, moisturizing agents, or coloring agents. Sunscreen compounds include those organic and inorganic materials employed to block ultraviolet light. Illustrative organic sunscreen compounds are derivatives of p-aminobenzoic acid (PABA), cinnamate, salicylate, benzophenones, anthranilates, dibenzoylmethanes, and camphores; examples or inorganic sunscreen compounds include zinc oxide and titanium dioxide. For example, octyl methoxycinnamate and 2-hydroxy-4-methoxy benzophenone (also known as oxybenzone) can be used. Octyl methoxycinnamate and 2-hydroxy-4-methoxy benzophenone are commercially available under the trademarks Parsol MCX and Benzophenone-3, respectively. Other examples include 2-phenylbenzimidazole-5-sulfonic acid (commercially available as Eusolex 232 from Rona), and octyldimethyl p-amino benzoic acid (octyl dimethyl PABA commercially available from Haarmann & Reimer). The exact amount of sunscreen employed in the emulsions can vary depending upon the degree of protection desired from the sun's UV radiation.

In general, the compounds of the invention are present in a topical formulation in an amount ranging from about 0.001% to about 5.0% by weight (10 ng to 50 μg per mg), based upon the total weight of the composition. Preferably, the compounds of the invention are present in an amount ranging from about 0.05% to about 3.0% by weight and, most preferably, the compounds are present in an amount ranging from about 0.05% to about 1.0% by weight (0.5 μg to 10 μg per mg).

For any compound described herein a therapeutically effective amount can be initially determined from cell culture assays. For instance the effective amount of immunostimulatory nucleic acid useful for inducing B-cell activation can be assessed using the in vitro assays with respect to stimulation index in comparison to known immunostimulatory acids. The stimulation index can be used to determine an effective amount of the particular oligonucleotide for the particular subject, and the dosage can be adjusted upwards or downwards to achieve the desired levels in the subject. Therapeutically effective amounts can also be determined from animal models. An animal model for psoriasis is provided by U.S. Pat. No. 5,945,576. A therapeutically effective dose can also be determined from human data for immunostimulatory nucleic acids which have been tested in humans (human clinical trials have been initiated) and for compounds which are known to exhibit similar pharmacological activities, such as other adjuvants, e.g., LT and other antigens for vaccination purposes. The applied dose can be adjusted based on the relative bioavailability and potency of the administered compound. Adjusting the dose to achieve maximal efficacy based on the methods described above and other methods as are well known in the art is well within the capabilities of the ordinarily skilled artisan. Most of the non-allergic inflammatory disease medicaments have been identified. These amounts can be adjusted when they are combined with immunostimulatory nucleic acids by routine experimentation.

The formulations of the invention are administered in pharmaceutically acceptable solutions, which may routinely contain pharmaceutically acceptable concentrations of salt, buffering agents, preservatives, compatible carriers, adjuvants, and optionally other therapeutic ingredients.

Immunostimulatory nucleic acids and non-allergic inflammatory disease medicaments and can be administered by any ordinary route for administering medications. Preferably, they are ingested, administered by systemic routes, topically applied, or inhaled. Systemic routes include oral and parenteral. Inhaled medications are preferred in some embodiments if direct delivery to the lung is desired, for example when lung is the site of non-allergic inflammation. Several types of metered dose inhalers are regularly used for administration by inhalation. These types of devices include metered dose inhalers (MDI), breath-actuated MDI, dry powder inhaler (DPI), spacer/holding chambers in combination with MDI, and nebulizers.

For use in therapy, an effective amount of the immunostimulatory nucleic acid can be administered to a subject by any mode that delivers the nucleic acid either systemically or to the desired surface, e.g., skin or mucosa. “Administering” the pharmaceutical composition of the present invention may be accomplished by any means known to the skilled artisan. Preferred routes of administration include but are not limited to oral, parenteral, intravenous, intramuscular, intracutaneous, subcutaneous, intradermal, subdermal, transdermal, topical, sublingual, intranasal, intratracheal, inhalation, ocular, vaginal, and rectal.

For oral administration, the compounds (i.e., immunostimulatory nucleic acids, non-allergic inflammatory disease medicament, other therapeutic agent) can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the crosslinked polyvinylpyrrolidone, agar, or alginic acid or a salt thereof such as sodium alginate. Optionally the oral formulations may also be formulated in saline or buffers for neutralizing internal acid conditions or may be administered without any carriers.

Dragee cores are provided with suitable coatings. For this purpose, concentrated sugar solutions may be used, which may optionally contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch. Techniques for preparing aerosol delivery systems are well known to those of skill in the art. Generally, such systems should utilize components which will not significantly impair the biological properties of the therapeutic, such as the immunostimulatory capacity of the nucleic acids (see, for example, Sciarra and Cutie, “Aerosols,” in Remington's Pharmaceutical Sciences, 18th edition, 1990, pp 1694-1712; incorporated by reference). Those of skill in the art can readily determine the various parameters and conditions for producing aerosols without resort to undue experimentation.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

In some embodiments, topical administration is preferred. For topical administration to the skin, the compounds according to the invention may be formulated as ointments, gels, creams, lotions, or as a transdermal patch for iontophoresis. One method for accomplishing topical administration includes transdermal administration, such as by iontophoresis. Iontophoretic transmission can be accomplished by using commercially available patches which deliver a compound continuously through unbroken skin for periods of hours to days to weeks, depending on the particular patch. This method allows for the controlled delivery of the immunostimulatory nucleic acid and/or non-allergic inflammatory disease medicament through the skin in relatively high concentrations. One example of an iontophoretic patch is the LECTRO PATCH™ sold by General Medical Company of Los Angeles, Calif. The patch provides dosages of different concentrations which can be continuously or periodically administered across the skin using electronic stimulation of reservoirs containing the immunostimulatory nucleic acid and/or non-allergic inflammatory disease medicament.

Topical administration also includes epidermal administration which involves the mechanical or chemical irritation of the outermost layer of the epidermis sufficiently to provoke an immune response to the irritant. The irritant attracts antigen presenting cells (APCs) to the site of irritation where they can then take up the immunostimulatory nucleic acid and/or non-allergic inflammatory disease medicament. One example of a mechanical irritant is a tyne-containing device. Such a device, for instance, the MONO-VACC® manufactured by Pasteur Merieux of Lyon, France, contains a plurality of tynes which irritate the skin and deliver the drug at the same time. The device contains a syringe plunger at one end and a tyne disk at the other. The tyne disk supports several narrow diameter tynes which are capable of scratching the outermost layer of epidermal cells. Chemical irritants include, for instance, keratinolytic agents such as salicylic acid, and can be used alone or in conjunction with mechanical irritants.

For topical administration to the skin, ointments, gels creams, and lotions can be formulated with an aqueous or oily base alone or together with suitable thickening and/or gelling agents. Lotions can be formulated with an aqueous or oily base and, typically, further include one or more emulsifying agents, stabilizing agents, dispersing agents, suspending agents, thickening agents, or coloring agents. (See, e.g., U.S. Pat. No. 5,563,153, entitled “Sterile Topical Anesthetic Gel”, issued to Mueller, D., et al., for a description of a pharmaceutically acceptable gel-based topical carrier.) The ointments, gels, creams, or lotions can optionally be formulated to include sunscreen compounds, fragrance, moisturizing agents, or coloring agents. Sunscreen compounds include those organic and inorganic materials employed to block ultraviolet light. Illustrative organic sunscreen compounds are derivatives of p-aminobenzoic acid (PABA), cinnamate, salicylate, benzophenones, anthranilates, dibenzoylmethanes, and camphores; examples or inorganic sunscreen compounds include zinc oxide and titanium dioxide. For example, octyl methoxycinnamate and 2-hydroxy-4-methoxy benzophenone (also known as oxybenzone) can be used. Octyl methoxycinnamate and 2-hydroxy-4-methoxy benzophenone are commercially available under the trademarks Parsol MCX and Benzophenone-3, respectively. Other examples include 2-phenylbenzimidazole-5-sulfonic acid (commercially available as Eusolex 232 from Rona), and octyldimethyl p-amino benzoic acid (octyl dimethyl PABA commercially available from Haarmann & Reimer). The exact amount of sunscreen employed in the emulsions can vary depending upon the degree of protection desired from the sun's UV radiation.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

In addition to the formulations described previously, the compounds may also be formulated as a depot preparation. Such long-acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer R (1990) Science 249:1527-33, which is incorporated herein by reference.

The immunostimulatory nucleic acids and non-allergic inflammatory disease medicament may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: acetic, benzene sulphonic, citric, formic, hydrobromic, hydrochloric, maleic, malonic, methane sulphonic, naphthalene-2-sulphonic, nitric, p-toluene sulphonic, phosphoric, salicylic, succinic, sulfuric, and tartaric. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

The pharmaceutical compositions of the invention contain an effective amount of an immunostimulatory nucleic acid and optionally non-allergic inflammatory disease medicament and/or other therapeutic agents optionally included in a pharmaceutically acceptable carrier. The term “pharmaceutically acceptable carrier” means one or more compatible solid or liquid fillers, dilutants or encapsulating substances which are suitable for administration to a human or other vertebrate animal. The term “carrier” denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficacy.

### EXAMPLES

### Methods

Mice and cell lines. Female C57BL/6, BALB/c and SCID-BALB/c mice at 6-12 weeks of age were purchased from the Jackson Laboratory (Bar Harbor, Me.). Mice were maintained in micro-isolator cages under specific pathogen-free conditions at the animal care facility at the University of Iowa. The RAW 264.7 murine macrophage cell line was a kind gift of J. Cowdery (University of Iowa).

Reagents. Nuclease-resistant phosphorothioate-modified ODN were provided by Coley Pharmaceutical Group (Wellesley, Mass.). The immunostimulatory oligonucleotides ODN 1826 (SEQ ID NO:63) and ODN 1585 (SEQ ID NO:496) and the non-stimulatory control ODN 1982 (SEQ ID NO:217) were used for these experiments. Ballas Z K et al. (1996) J Immunol 157:1840-45; Klinman D M et al. (1996) Proc Natl Acad Sci USA 93:2879-83. Escherichia coli (strain B) DNA and calf thymus DNA were purchased from Sigma (St. Louis, Mo.). All DNA and ODN were purified by extraction with phenol:chloroform:isoamyl alcohol (25:24:1). The endotoxin level in the DNA and ODN was <1.7 ng/mg as assayed with Limulus amebocyte lysate QCL-1000 (Biowhittaker, Walkersville, Md.). LPS from Escherichia coli (serotype 0111 :B4) was obtained from Difco (Detroit, Mich.) and resuspended in pyrogen-free saline. Rabbit polyclonal anti-murine COX-2 was obtained from Cayman Chemical (Ann Arbor, Mich.); rabbit polyclonal anti-COX-1 was obtained from Santa Cruz Biotechnology (Santa Cruz, Calif.). SC-58236, mouse neutralizing anti-PGE₂ (2B5) and isotype control antibody (MOPC21) were kindly provided by J. Portanova (Pharmacia, St. Louis, Mo.). Piroxicam and NS-398 were obtained from Biomol (Plymouth Meeting, Mass.). Arachidonic acid and PGE₂ were obtained from Cayman Chemical.

Cell culture conditions. RAW 264.7 cells and murine spleen cells were cultured in RPMI 1640 supplemented with 10% FCS, 2 mM L-glutamine, 0.05 mM 2-mercaptoethanol, penicillin (100 U/ml) and streptomycin (100 U/ml) in 12-well tissue culture plates (Costar, Corning, N.Y.). Cells were incubated in media alone or media supplemented with ODN. Supernatants from triplicate cultures were harvested and stored at −70° C. before analysis for PG or cytokine concentration. Cells were subsequently harvested for either RNA or protein isolation. In some cultures, ODN-stimulated spleen cells were incubated in the presence of PGE₂ (0.1 μM), piroxicam (16 μM), SC-58236 (0.125 μM), anti-PGE₂ antibody 2B5 (6.7 μg/ml), or isotype antibody MOPC21 (6.7 μg/ml). For quantitative analysis of macrophage PG production, RAW 264.4 cells were cultured at 1×10⁵ cells/ml in media or media with ODN (3 μg/ml). After 24 h of culture the supernatant was removed and cells were incubated in PBS supplemented with arachidonic acid (10 μM). In some cultures, the COX-2 specific inhibitors NS-398 (10 μM) or SC-58236 (0.125 μM) were added.

PG quantification. Quantification of PGE₂ levels in tissue culture supernatants were determined using the PGE₂ EIA kit from Cayman Chemical, as per the manufacturer's instructions.

RNase protection assay. A murine COX-2 cDNA fragment (nucleotides 205-505; GenBank accession no. M88242) was synthesized by RT-PCR using mouse brain RNA as a template and cloned into pGEM-4. Fragments of the RPL32-4A gene were also cloned into pGEM-4. Dudov KP et al. (1984) Cell 37:457-68. L32 served as an internal loading control. RNase protection assay (RPA) for the detection of COX-2 was performed as previously described. Stadler A et al. (1998) In: Neurodegeneration Methods and Protocols, Harry J and Tilson H A, eds., Humana Press, Totowa, N.J., p. 53. Briefly, for the synthesis of a ³²P-radiolabeled anti-sense RNA probe, equimolar mixtures of the linearized COX-2 and L32 templates were used. Hybridization reactions were performed overnight at 56° C. Following RNase digestion, the RNA duplexes were isolated by electrophoresis in a standard 7.5% acrylamide/12 M ureal0.5% TBE sequencing gel. Dried gels were placed on BMR film and were exposed at −70° C.

Western blotting. Protein was isolated from cultured cells by resuspending in lysis buffer [50 mM Tris (hydroxymethyl) aminomethane, pH 7.5, 150 mM NaCl, 100 μg/ml PMSF, 1 μg/ml aprotinin, 1 μg/ml leupeptin, 1 mM diethyldithiocarbamic acid, 1% NP-40, and 1% sodium deoxycholate]. Cells were lysed by sonication (20 s, 4° C.). Debris was eliminated by centrifugation (15 min, 1000 g). Protein concentration was measured using a commercial reagent based on BCA staining (Pierce, Rockford, Ill.), using BSA as an internal standard. Equal amounts of cellular protein were loaded onto a 10% polyacrylamide gel and separated by electrophoresis (200 V for 45 min). Proteins were then transferred to nitrocellulose (100 V for 1 h) and the membrane was blocked with 5% non-fat dry milk. The nitrocellulose was then incubated with a rabbit polyclonal primary antibody (anti-COX-2, 1:1000, Cayman; anti-COX-1, 1:1000, Santa Cruz) overnight at 4° C. Antibody labeling was detected using enhanced chemiluminescence (ECL; Amersham) as per the manufacturer's instructions. Specificity of the anti-cyclooxygenase antibody was confirmed with the use of ram seminal vesicle PGHS-1 (COX-1, Oxford Biomedical Research, Oxford, Mich.) and sheep placenta PGHS-2 (COX-2, Cayman).

Films and photographs of RPA or Western blots were scanned in at 6000 DPI using an Epson Expression 1600 scanner. Densitometric analysis was performed using Vtrace (developed at the University of Iowa Image Analysis Facility) operating on a SGI O2 Workstation. Average and integrated OD measurements were made on user-selected regions. A Kodak photographic step tablet was used to calibrate optical density.

Cytokine quantification. IFN-γ detection was performed using a cytokine ELISA kit from PharMingen (San Diego, Calif.). Immulon-1B microtiter plates were obtained from Dynatech (Chantilly, Calif.). After incubation with peroxidase substrate (tetramethylbenzidine) the plates were read at 650 nm on a microplate reader (Cambridge Technology, Watertown, Mass.).

Cytotoxicity assay. Spleen cells from C57BL/6 mice were depleted of B cells using paramagnetic beads coated with goat anti-mouse Ig as previously described. Ballas Z K et al. (1990) J Immunol 145:1039-45; Ballas Z K et al. (1993) J Immunol 150:17-30. Murine spleen cells were cultured at 5×10⁶ cells/ml, at 37° C. in a 5% CO₂ humidified atmosphere in 24 well-plates with medium alone or with ODN (10 μg/ml). Where indicated, cultures were supplemented with piroxicam (0.1 mM), SC-58236 (0.25 μM) or PGE₂ (0.1 μM). Cultures were harvested at 18 h and the cells were used as effectors in a standard 4-h ⁵¹Cr-release assay against YAC-1 target cells labeled with Na⁵¹CrO₄ (Amersham Life Science, Arlington Heights, Ill.) as described previously. Ballas Z K et al. (1990) J Immunol 145:1039-45; Ballas Z K et al. (1993) J Immunol 150:17-30; Ballas Z K et al. (1990) J Allergy Clin Immunol 85:453-39. One lytic unit (LU) was defined as the number of the cells needed to exert 30% specific lysis.

In vivo experiments. BALB/c mice were injected i.p. with 0.2 ml of diluent or SC-58236 (20 mg/kg). One hour later, mice were injected with 30 μg of ODN 1826 or PBS. Five hours later, the mice were anaesthetized with Avertin (Aldrich, Milwaukee, Wis.) and blood was obtained from the retro-orbital plexus. The blood was allowed to clot on ice for 1 h and centrifuged at 10,000 r.p.m. for 10 min. The serum was used for IFN-γ determination by ELISA (as described above).

### Example 1

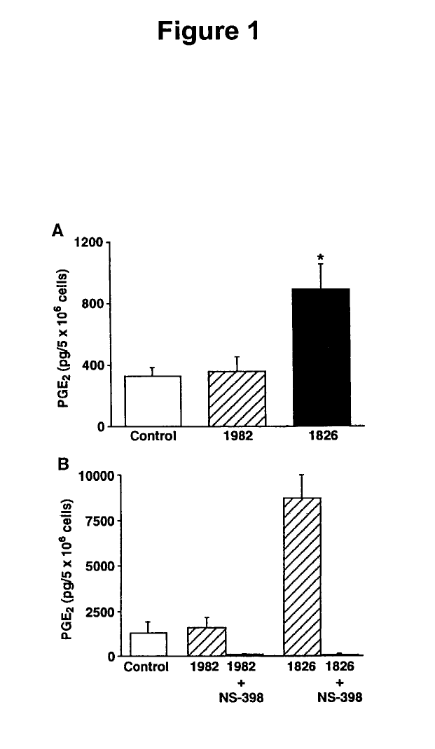

Characterization of PG production induced by immunostimulatory DNA. Presence of immunostimulatory ODN resulted in increased PGE₂ production from both spleen cells (2.5-fold greater than control ODN; FIG. 1A) and the RAW macrophage cell line (5.4-fold greater than control ODN; FIG. 1B). The PGE₂ produced was derived from COX-2 enzymatic activity as the COX-2 selective ODN-stimulated cells. Similar results were also obtained using the COX-2-selective inhibitor SC-58236. HPLC analysis of ODN-stimulated RAW cells demonstrated that PGE₂ was the dominant eicosanoid induced by CpG DNA.

### Example 2

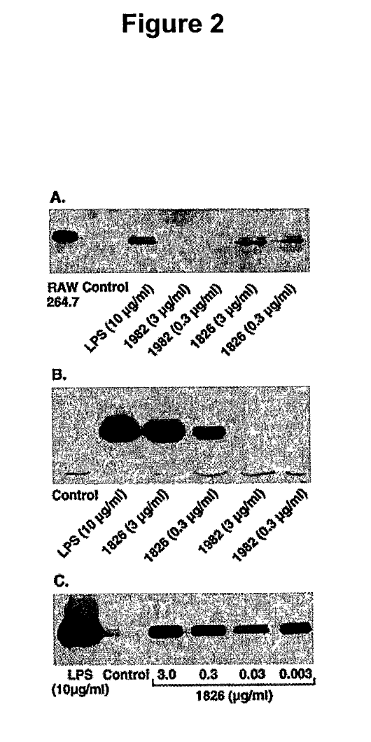

Immunostimulatory ODN induce COX-2 mRNA. RAW 264.7 macrophages were incubated with media or ODN (3 μg/ml) for varying time periods (0, 2, 4, 6 and 24 h) and RNA isolated for COX-2 expression using RPA. As shown in FIG. 2B, stimulatory ODN 1826 effectively increased COX-2 mRNA (6 h level was 21-fold greater than 0 h). In contrast, no significant increase in COX-2 message was seen using the non-stimulatory ODN 1982 (FIG. 2A). This analysis also demonstrated that COX-2 mRNA was rapidly induced in response to stimulatory ODN. Within 2 h the stimulatory ODN 1826 induced COX-2 mRNA expression and the COX-2 mRNA levels remained elevated over the 24-h time period (FIG. 2B).

### Example 3

Immunostimulatory ODN induces COX-2 protein expression. Spleen cells incubated with stimulatory ODN had increased expression of COX-2 protein, whereas non-stimulatory ODN did not induce COX-2 expression. Stimulation of RAW 264.7 cells with stimulatory ODN 1826 also resulted in a marked induction of COX-2 protein, while no induction was seen using the control ODN 1982. Stimulatory ODN was an extremely potent inducer of COX-2 protein in RAW 264.7 macrophages as amounts as low as 3 ng/ml effectively induced COX-2 protein expression. In contrast, neither stimulatory nor control ODN altered the level of protein expression of COX-1.

### Example 4

COX inhibitors enhance IFN-γ secretion induced by CpG DNA. CpG DNA is known to elicit strong Th1-like immune responses both in vitro and in vivo. Klinman D M et al. (1996) Proc Natl Acad Sci USA 93:2879-83. In contrast, PGE₂, which is induced by CpG DNA, can inhibit Th1 responses. Betz M et al. (1991) J Immunol 146:108-13. Murine spleen cells were stimulated with CpG DNA in the presence of piroxicam, a non-selective COX inhibitor, or SC-58236, a selective inhibitor of COX-2, and quantified IFN-γ production. As shown in Table 3, inhibition of COX-2 by SC-58236 resulted in a 2-fold enhancement of IFN-γ secretion from CpG DNA-stimulated spleen cells. Similar results were obtained with the non-selective inhibitor, piroxicam. Neither COX inhibitor alone stimulated IFN-γ secretion. The finding that SC-58236, a COX-2 selective inhibitor, enhanced IFN-γ secretion indicated that the enhanced IFN-γ production is secondary to inhibition of COX-2 derived PG.

### Example 5

Inhibition of PGE₂ synthesis plays an important role in enhancing IFN-γ secretion. The blockade of the COX enzyme will inhibit synthesis of multiple PG (e.g., PGE₂, PGD₂ and dPGJ₂), all of which potentially can inhibit Th1 immune responses. To assess the role of PGE₂ in the modulation of IFN-γ secretion elicited by CpG ODN, a neutralizing antibody (2B5) specific for PGE₂ was added into CpG DNA-stimulated spleen cell cultures. Compared with control antibody, 2B5 significantly enhanced IFN-γ secretion from CpG-stimulated spleen cells (Table 3). The enhancement was similar in magnitude to that observed when COX inhibitors were added to the cultures (Table 3). These data suggest that inhibition of PGE₂ synthesis is the mechanism by which the COX inhibitors enhance IFN-γ secretion.

TABLE 3
Enhancement of IFN-γ secretion by selective COX-2 inhibitor SC-58236^{a}

| Stimulus | IFN-γ (pg/mL) |
|---|---|
| None | 0 ± 5 |
| 1826 | 1960 ± 247 |
| Diluent | 10 ± 46 |
| 1826 + diluent | 2101 ± 198 |
| 1826 + SC-58236 | 4186 ± 304^{b} |
| 1826 + PGE₂ | 450 ± 246 |
| 1826 + SC-58236 + PGE₂ | 425 ± 95 |
| SC-58236 | 0 ± 2 |
| PGE₂ | 0 ± 4 |
| SC-58236 + PGE₂ | 0 ± 7 |
| 1826 + 2B5 | 5525 ± 769^{c} |
| 2B5 | 0 ± 7 |
| 1826 + MOPC21 | 2757 ± 125 |
| MOPC21 | 0 ± 2 |

^{a}BALB/c mouse spleen cells were preincubated with the following reagents at the indicated concentrations: SC-58236 (0.125 μM), PGE₂ (0.1 μM), PGE₂-neutralizing antibody 2B5 (6.7 μg/mL) or control antibody (6.7 μg/mL) for 30 min. The spleen cells were subsequently stimulated with CpG ODN 1826 (0.3 μg/mL) for 24 h. IFN-γ concentration in the supernatant was measured by ELISA (PharMingen).
^{b}Statistically significant as compared with control (1826 + diluent; P < 0.05, Student's t-test)
^{c}Statistically significant as compared with control (1826 + MOPC21; P < 0.05, Student's t-test)

### Example 6

COX inhibitors augment the cytotoxic activity of NK cells. It has previously been reported that certain CpG DNA sequences can elicit strong lytic activity in NK cells (Ballas Z K et al. (1996) J Immunol 157:1840-45), whereas exogenous PGE₂ is known to inhibit the activity of NK cell killing (Kanar M C et al. (1988) J Clin Immunol 8:69-79; Young M R et al. (1986) J Natl Cancer Inst 77:425-29). B cell-depleted murine spleen cells were stimulated with the stimulatory ODN 1585 in the presence or absence of a COX inhibitor. Incubation of spleen cells with the COX-2 selective inhibitor SC-58236 enhanced the NK activity induced by CpG DNA (Table 4). Similar data were obtained using the non-selective COX-inhibitor, piroxicam. The addition of exogenous PGE₂ abolished the lytic activity of NK cells (Table 4). Taken together, these data suggest that blockade of COX-2-derived PGE₂ production resulted in enhancement of CpG DNA-induced NK lytic activity.

TABLE 5
Enhancement of NK activity of selective COX-2 inhibitor^{a}

| Stimulus | Mouse Spleen Cells (LU/10⁶) |
|---|---|
| Medium alone | 0.0 |
| IL-2 | 13.9 |
| 1585 | 7.3 |
| 1585 + SC-58236 | 12.2 |
| 1585 + SC-58236 + PGE₂ | 1.7 |
| SC-58236 | 0.0 |
| PGE₂ | 0.0 |
| SC-58236 + PGE₂ | 0.0 |
| ^{a}C57BL/6 mouse spleen cells were depleted of B cells using paramagnetic beads coated with goat anti-mouse Ig. The spleen cells were then incubated with or without the following reagents at the indicated concentrations: IL-2 (100 U/mL), CpG ODN 1585 (SEQ ID NO:496; 5 μg/mL), SC-58236 (0.25 μg/mL), and PGE₂ (0.1 μg/mL) for 18 h. Lytic activity was tested on ⁵¹Cr-labeled YAC-1 cells in a standard 4-h ⁵¹Cr-release assay. One lytic unit (LU) was defined as the number of | |
| # effectors that causes 30% of ⁵¹Cr release. | |

### Example 7

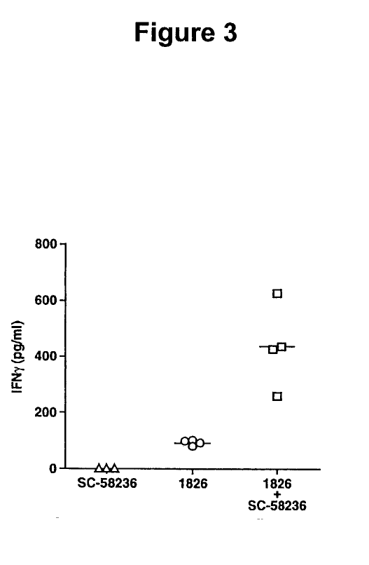

COX inhibitors augment IFN-γ production in vivo. To determine whether COX inhibitors could also enhance the immune stimulatory effects of CpG-DNA in vivo, mice were treated with COX inhibitors (piroxicam or SC-58236) and subsequently injected with stimulatory ODN 1826. Serum IFN-γ levels were quantified 5 h post ODN injection. Inhibition of COX-2 with the COX-2 selective inhibitor SC-58236 resulted in a 4.6-fold increase in IFN-γ secretion above that seen in mice injected with ODN 1826 alone (FIG. 3). Similar results were seen when mice were treated with piroxicam, a non-selective COX inhibitor. COX inhibition resulted in increased IFN-γ production in SCID/BALB/c mice treated with ODN 1826, suggesting that cells other than T or B cells are responsible for the enhancement of CpG-induced IFN-γ secretion by the COX-2 inhibitor.

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

All references, patents and patent publications that are recited in this application are incorporated in their entirety herein by reference.

We claim:

1. A method for treating a non-allergic inflammatory disease, comprising:
administering to a subject having or at risk of developing a non-allergic inflammatory disease a therapeutically effective amount of an immunostimulatory nucleic acid to treat or prevent the non-allergic inflammatory disease.

2. The method of claim 1, wherein the therapeutically effective amount of the immunostimulatory nucleic acid reduces or prevents non-allergic inflammation in a tissue of the subject.

3. The method of claim 1, wherein the non-allergic inflammatory disease involves an epithelium.

4. The method of claim 1, wherein the non-allergic inflammatory disease involves a mucosal epithelium.

5. The method of claim 1, wherein the non-allergic inflammatory disease is selected from the group consisting of: psoriasis, eczema, allergic contact dermatitis, latex dermatitis, and inflammatory bowel disease.

6. The method of claim 1, wherein the non-allergic inflammatory disease is psoriasis.

7. The method of claim 1, wherein the non-allergic inflammatory disease is allergic contact dermatitis.

8. The method of claim 1, wherein the non-allergic inflammatory disease is latex dermatitis.

9. The method of claim 1, wherein the immunostimulatory nucleic acid is a CpG nucleic acid.

10. The method of claim 1, wherein the immunostimulatory nucleic acid is a methylated CpG nucleic acid.

11. The method of claim 1, wherein the immunostimulatory nucleic acid is a T-rich nucleic acid.

12. The method of claim 1, wherein the immunostimulatory nucleic acid is a poly-G nucleic acid.

13. The method of claim 12, wherein the poly-G nucleic acid comprises the formula
5′-X₁X₂GGGX₃X₄-3′
wherein each of X₁, X₂, X₃, and X₄ is any nucleotide other than G.

14. The method of claim 13, wherein the poly-G nucleic acid does not include any of the formulas 5′-GXGGG-3′, 5′-XGGGG-3′, or 5′-GGGXG-3′, wherein X is any nucleotide.

15. The method of claim 1, wherein the immunostimulatory nucleic acid is a synthetic nucleic acid.

16. The method of claim 1, wherein the immunostimulatory nucleic acid comprises at least one stabilized internucleotide linkage.

17. The method of claim 16, wherein the stabilized internucleotide linkage is a phosphorothioate linkage.

18. The method of claim 1, wherein the immunostimulatory nucleic acid has a backbone completely made up of stabilized internucleotide linkages.

19. The method of claim 1, wherein the immunostimulatory nucleic acid comprises between 6 and 100 nucleotides.

20. The method of claim 1, wherein the immunostimulatory nucleic acid comprises between 8 and 40 nucleotides.

21. The method of claim 1, wherein the immunostimulatory nucleic acid induces IL-12.

22. The method of claim 1, wherein the immunostimulatory nucleic acid induces IFN-α.

23. The method of claim 1, wherein the immunostimulatory nucleic acid induces IFN-γ.

24. The method of claim 1, wherein the immunostimulatory nucleic acid induces IL-10.

25. The method of claim 1, wherein the immunostimulatory nucleic acid is administered locally to damaged epithelium.

26. The method of claim 1, wherein the immunostimulatory nucleic acid is administered locally to intact epithelium.

27. The method of claim 1, wherein the immunostimulatory nucleic acid is administered systemically.

28. The method of claim 1, wherein the immunostimulatory nucleic acid is administered orally.

29. The method of claim 1, wherein the immunostimulatory nucleic acid is administered parenterally.

30. The method of claim 1, wherein the immunostimulatory nucleic acid is administered topically.

31. The method of claim 1, wherein the immunostimulatory nucleic acid is administered transdermally.

32. The method of claim 1, further comprising administering to the subject an anti-inflammatory agent selected from the group consisting of: anti-inflammatory corticosteroids, nonsteroidal anti-inflammatory drugs, vitamin A analogs, vitamin D analogs, retinoids, cytokines, agonists of cytokines, antagonists of cytokines, agonists of cytokine receptors, antagonists of cytokine receptors, cytokine receptor analogs, antibodies specific for cytokines, antibodies specific for cytokine receptors, and immunosuppressive agents.

33. A method for treating inflammatory bowel disease, comprising:
administering to a subject having or at risk of developing an inflammatory bowel disease a therapeutically effective amount of an immunostimulatory nucleic acid to treat or prevent the inflammatory bowel disease.

34. The method of claim 33, wherein the inflammatory bowel disease is ulcerative colitis.

35. The method of claim 33, wherein the inflammatory bowel disease is Crohn's disease.

36. The method of claim 33, wherein the immunostimulatory nucleic acid is a CpG nucleic acid.

37. The method of claim 33, wherein the immunostimulatory nucleic acid is a methylated CpG nucleic acid.

38. The method of claim 33, wherein the immunostimulatory nucleic acid is a T-rich nucleic acid.

39. The method of claim 33, wherein the immunostimulatory nucleic acid is a poly-G nucleic acid.

40. The method of claim 39, wherein the poly-G nucleic acid comprises the formula
5′-X₁X₂GGGX₃X₄-3′
wherein each of X₁, X₂, X₃, and X₄ is any nucleotide other than G.

41. The method of claim 40, wherein the poly-G nucleic acid does not include any of the formulas 5′-GXGGG-3′, 5′-XGGGG-3′, or 5′-GGGXG-3′, wherein X is any nucleotide.

42. The method of claim 33, wherein the immunostimulatory nucleic acid is a synthetic nucleic acid.

43. The method of claim 33, wherein the immunostimulatory nucleic acid comprises at least one stabilized internucleotide linkage.

44. The method of claim 43, wherein the stabilized internucleotide linkage is a phosphorothioate linkage.

45. The method of claim 33, wherein the immunostimulatory nucleic acid has a backbone completely made up of stabilized internucleotide linkages.

46. The method of claim 33, wherein the immunostimulatory nucleic acid comprises between 6 and 100 nucleotides.

47. The method of claim 33, wherein the immunostimulatory nucleic acid comprises between 8 and 40 nucleotides.

48. The method of claim 33, wherein the immunostimulatory nucleic acid induces IL-12.

49. The method of claim 33, wherein the immunostimulatory nucleic acid induces IFN-α.

50. The method of claim 33, wherein the immunostimulatory nucleic acid induces IFN-γ.

51. The method of claim 33, wherein the immunostimulatory nucleic acid induces IL-10.

52. The method of claim 33, wherein the immunostimulatory nucleic acid is administered locally to damaged mucosal epithelium.

53. The method of claim 33, wherein the immunostimulatory nucleic acid is administered locally to intact mucosal epithelium.

54. The method of claim 33, wherein the immunostimulatory nucleic acid is administered systemically.

55. The method of claim 33, wherein the immunostimulatory nucleic acid is administered orally.

56. The method of claim 33, wherein the immunostimulatory nucleic acid is administered rectally.

57. The method of claim 33, wherein the composition is administered parenterally.

58. The method of claim 33, further comprising administering to the subject an anti-inflammatory agent selected from the group consisting of: 5-aminosalicylate, agents containing 5-aminosalicylate, anti-inflammatory corticosteroids, nonsteroidal anti-inflammatory drugs, cytokines, agonists of cytokines, antagonists of cytokines, agonists of cytokine receptors, antagonists of cytokine receptors, cytokine receptor analogs, antibodies specific for cytokines, antibodies specific for cytokine receptors, and immunosuppressive agents.

59. The method of claim 33, further comprising administering to the subject an anti-inflammatory agent selected from the group consisting of: 5-aminosalicylate and agents containing 5-aminosalicylate.

60. A pharmaceutical composition, comprising:
an immunostimulatory nucleic acid in an effective amount for preventing or treating an immune or inflammatory response associated with a non-allergic inflammatory disease,
a non-allergic inflammatory disease medicament, and
a pharmaceutically acceptable carrier.

61. The pharmaceutical composition of claim 60, wherein the non-allergic inflammatory disease is selected from the group consisting of: psoriasis, eczema, allergic contact dermatitis, latex dermatitis, and inflammatory bowel disease.

62. The pharmaceutical composition of claim 60, wherein the immunostimulatory nucleic acid is a CpG nucleic acid.

63. The pharmaceutical composition of claim 60, wherein the immunostimulatory nucleic acid is a methylated CpG nucleic acid.

64. The pharmaceutical composition of claim 60, wherein the immunostimulatory nucleic acid is a T-rich nucleic acid.

65. The pharmaceutical composition of claim 60, wherein the immunostimulatory nucleic acid is a poly-G nucleic acid.

66. The pharmaceutical composition of claim 65, wherein the poly-G nucleic acid comprises the formula
5′-X₁X₂GGGX₃X₄-3′
wherein each of X₁, X₂, X₃, and X₄ is any nucleotide other than G.

67. The pharmaceutical composition of claim 66, wherein the poly-G nucleic acid does not include any of the formulas 5′-GXGGG-3′, 5′-XGGGG-3′, or 5′-GGGXG-3′, wherein X is any nucleotide.

68. The pharmaceutical composition of claim 60, wherein the immunostimulatory nucleic acid is a synthetic nucleic acid.

69. The pharmaceutical composition of claim 60, wherein the immunostimulatory nucleic acid comprises at least one stabilized internucleotide linkage.

70. The pharmaceutical composition of claim 69, wherein the stabilized internucleotide linkage is a phosphorothioate linkage.

71. The pharmaceutical composition of claim 60, wherein the immunostimulatory nucleic acid has a backbone completely made up of stabilized internucleotide linkages.

72. The pharmaceutical composition of claim 60, wherein the immunostimulatory nucleic acid comprises between 6 and 100 nucleotides.

73. The pharmaceutical composition of claim 60, wherein the immunostimulatory nucleic acid comprises between 8 and 40 nucleotides.

74. The pharmaceutical composition of claim 60, wherein the non-allergic inflammatory disease medicament is selected from the group consisting of: 5-aminosalicylate, agents containing 5-aminosalicylate, anti-inflammatory corticosteroids, nonsteroidal anti-inflammatory drugs, vitamin A analogs, vitamin D analogs, retinoids, cytokines, agonists of cytokines, antagonists of cytokines, agonists of cytokine receptors, antagonists of cytokine receptors, cytokine receptor analogs, antibodies specific for cytokines, antibodies specific for cytokine receptors, and immunosuppressive agents.

75. A pharmaceutical composition, comprising:
an immunostimulatory nucleic acid in an effective amount for preventing or treating an immune or inflammatory response associated with a non-allergic inflammatory disease, and
a pharmaceutically acceptable carrier, wherein the immunostimulatory nucleic acid and pharmaceutically acceptable carrier are prepared in a formulation selected from the group consisting of: a lotion, a cream, an ointment, and a gel.

76. The pharmaceutical composition of claim 75, wherein the non-allergic inflammatory disease is selected from the group consisting of: psoriasis, eczema, allergic contact dermatitis, and latex dermatitis.

77. The pharmaceutical composition of claim 75, wherein the non-allergic inflammatory disease is psoriasis.

78. The pharmaceutical composition of claim 75, wherein the non-allergic inflammatory disease is allergic contact dermatitis.

79. The pharmaceutical composition of claim 75, wherein the non-allergic inflammatory disease is latex dermatitis.

80. The pharmaceutical composition of claim 75, wherein the immunostimulatory nucleic acid is a CpG nucleic acid.

81. The pharmaceutical composition of claim 75, wherein the immunostimulatory nucleic acid is a methylated CpG nucleic acid.

82. The pharmaceutical composition of claim 75, wherein the immunostimulatory nucleic acid is a T-rich nucleic acid.

83. The pharmaceutical composition of claim 75, wherein the immunostimulatory nucleic acid is a poly-G nucleic acid.

84. The pharmaceutical composition of claim 83, wherein the poly-G nucleic acid comprises the formula
5′-X₁X₂GGGX₃X₄-3′
wherein each of X₁, X₂, X₃, and X₄ is any nucleotide other than G.

85. The pharmaceutical composition of claim 84, wherein the poly-G nucleic acid does not include any of the formulas 5′-GXGGG-3′, 5′-XGGGG-3′, or 5′-GGGXG-3′, wherein X is any nucleotide.

86. The pharmaceutical composition of claim 75, wherein the immunostimulatory nucleic acid is a synthetic nucleic acid.

87. The pharmaceutical composition of claim 75, wherein the immunostimulatory nucleic acid comprises at least one stabilized internucleotide linkage.

88. The pharmaceutical composition of claim 87, wherein the stabilized internucleotide linkage is a phosphorothioate linkage.

89. The pharmaceutical composition of claim 75, wherein the immunostimulatory nucleic acid has a backbone completely made up of stabilized internucleotide linkages.

90. The pharmaceutical composition of claim 75, wherein the immunostimulatory nucleic acid comprises between 6 and 100 nucleotides.

91. The pharmaceutical composition of claim 75, wherein the immunostimulatory nucleic acid comprises between 8 and 40 nucleotides.

92. A method of augmenting Th1-like immune activation induced by an immunostimulatory nucleic acid, comprising:
contacting an immune cell with an effective amount of an immunostimulatory nucleic acid to induce Th1-like immune activation; and
contacting the immune cell with an inhibitor of cyclooxygenase-2 (COX-2) expression, in an amount effective to augment Th1-like immune activation induced by the immunostimulatory nucleic acid.

93. The method of claim 92, wherein the immunostimulatory nucleic acid is administered to a subject in need of Th1-like immune activation in an effective amount to induce Th1-like immune activation, and wherein the inhibitor of COX-2 expression is administered to the subject in an effective amount to augment Th1-like immune activation induced by the immunostimulatory nucleic acid.

94. A method of augmenting Th1-like immune activation induced by an immunostimulatory nucleic acid, comprising:
contacting an immune cell with an effective amount of an immunostimulatory nucleic acid to induce Th1-like immune activation; and
contacting the immune cell with an agent that inhibits PGE₂ signaling through its receptor, in an amount effective to augment the Th1-like immune activation induced by the immunostimulatory nucleic acid.

95. The method of claim 94, wherein the agent that inhibits PGE₂ signaling through its receptor is an antibody that binds specifically to PGE₂.

96. The method of claim 94, wherein the immunostimulatory nucleic acid is administered to a subject in need of Th1-like immune activation in an effective amount to induce Th1-like immune activation, and wherein the agent that inhibits PGE₂ signaling through its receptor is administered to the subject in an effective amount to augment the Th1-like immune activation.

97. A method of augmenting Th1-like immune activation in a subject, comprising:
administering to a subject in need of Th1-like immune activation an effective amount of an immunostimulatory nucleic acid to induce Th1-like immune activation; and
administering to the subject an effective amount of a cyclooxygenase inhibitor to inhibit prostaglandin expression, wherein the subject is free of symptoms of asthma or allergy otherwise calling for treatment with immunostimulatory nucleic acid, and wherein Th1-like immune activation induced by administering the immunostimulatory nucleic acid and the cyclooxygenase inhibitor is greater than Th1-like immune activation induced by administering the immunostimulatory nucleic acid alone.

98. The method of claim 97, wherein the prostaglandin is PGE₂.

99. The method of claim 97, wherein the cyclooxygenase inhibitor is a nonsteroidal anti-inflammatory drug (NSAID).

100. The method of claim 97, wherein the cyclooxygenase inhibitor is a selective inhibitor of COX-2 catalytic activity.


SEQUENCE LISTING (1040 sequences)

SEQ ID NO: 1 - DNA, 18 bp, Artificial Sequence
  Synthetic Oligonucleotide
tctcccagcgtgcgccat

SEQ ID NO: 2 - DNA, 20 bp, Artificial Sequence
  Synthetic Oligonucleotide
ataatccagcttgaaccaag

SEQ ID NO: 3 - DNA, 20 bp, Artificial Sequence
  Synthetic Oligonucleotide
ataatcgacgttcaagcaag

SEQ ID NO: 4 - DNA, 18 bp, Artificial Sequence
  Synthetic Oligonucleotide
taccgcgtgcgaccctct

SEQ ID NO: 5 - DNA, 9 bp, Artificial Sequence
  Synthetic Oligonucleotide
ggggagggt

SEQ ID NO: 6 - DNA, 9 bp, Artificial Sequence
  Synthetic Oligonucleotide
ggggagggg

SEQ ID NO: 7 - DNA, 9 bp, Artificial Sequence
  Synthetic Oligonucleotide
ggtgaggtg

SEQ ID NO: 8 - DNA, 20 bp, Artificial Sequence
  Synthetic Oligonucleotide
  modified_base (8)...(8): m5c
tccatgtcgttcctgatgct

SEQ ID NO: 9 - DNA, 15 bp, Artificial Sequence
  Synthetic Oligonucleotide
  modified_base (11)...(11): m5c
gctaccttagcgtga

SEQ ID NO: 10 - DNA, 20 bp, Artificial Sequence
  Synthetic Oligonucleotide
  modified_base (8)...(8): m5c
tccatgacgttcctgatgct

SEQ ID NO: 11 - DNA, 20 bp, Artificial Sequence
  Synthetic Oligonucleotide
  modified_base (13)...(13): m5c
tccatgacgttcctgatgct

SEQ ID NO: 12 - DNA, 15 bp, Artificial Sequence
  Synthetic Oligonucleotide
  modified_base (7)...(7): m5c
gctagacgttagtgt

SEQ ID NO: 13 - DNA, 19 bp, Artificial Sequence
  Synthetic Oligonucleotide
agctccatggtgctcactg

SEQ ID NO: 14 - DNA, 20 bp, Artificial Sequence
  Synthetic Oligonucleotide
ccacgtcgaccctcaggcga

SEQ ID NO: 15 - DNA, 20 bp, Artificial Sequence
  Synthetic Oligonucleotide
gcacatcgtcccgcagccga

SEQ ID NO: 16 - DNA, 19 bp, Artificial Sequence
  Synthetic Oligonucleotide
gtcactcgtggtacctcga

SEQ ID NO: 17 - DNA, 25 bp, Artificial Sequence
  Synthetic Oligonucleotide
gttggatacaggccagactttgttg

SEQ ID NO: 18 - DNA, 25 bp, Artificial Sequence
  Synthetic Oligonucleotide
gattcaacttgcgctcatcttaggc

SEQ ID NO: 19 - DNA, 24 bp, Artificial Sequence
  Synthetic Oligonucleotide
accatggacgaactgtttcccctc

SEQ ID NO: 20 - DNA, 24 bp, Artificial Sequence
  Synthetic Oligonucleotide
accatggacgagctgtttcccctc

SEQ ID NO: 21 - DNA, 24 bp, Artificial Sequence
  Synthetic Oligonucleotide
accatggacgacctgtttcccctc

SEQ ID NO: 22 - DNA, 24 bp, Artificial Sequence
  Synthetic Oligonucleotide
accatggacgtactgtttcccctc

SEQ ID NO: 23 - DNA, 24 bp, Artificial Sequence
  Synthetic Oligonucleotide
accatggacggtctgtttcccctc

SEQ ID NO: 24 - DNA, 24 bp, Artificial Sequence
  Synthetic Oligonucleotide
accatggacgttctgtttcccctc

SEQ ID NO: 25 - DNA, 25 bp, Artificial Sequence
  Synthetic Oligonucleotide
ccactcacatctgctgctccacaag

SEQ ID NO: 26 - DNA, 25 bp, Artificial Sequence
  Synthetic Oligonucleotide
acttctcatagtccctttggtccag

SEQ ID NO: 27 - DNA, 20 bp, Artificial Sequence
  Synthetic Oligonucleotide
tccatgagcttcctgagtct

SEQ ID NO: 28 - DNA, 20 bp, Artificial Sequence
  Synthetic Oligonucleotide
  modified_base (9)...(9): I
  modified_base (11)...(11): I
  modified_base (15)...(15): I
gaggaaggngnggangacgt

SEQ ID NO: 29 - DNA, 20 bp, Artificial Sequence
  Synthetic Oligonucleotide
  modified_base (7)...(7): I
  modified_base (13)...(13): I
  modified_base (18)...(18): I
gtgaatncgttcncgggnct

SEQ ID NO: 30 - DNA, 6 bp, Artificial Sequence
  Synthetic Oligonucleotide
aaaaaa

SEQ ID NO: 31 - DNA, 6 bp, Artificial Sequence
  Synthetic Oligonucleotide
cccccc

SEQ ID NO: 32 - DNA, 6 bp, Artificial Sequence
  Synthetic Oligonucleotide
ctgtca

SEQ ID NO: 33 - DNA, 6 bp, Artificial Sequence
  Synthetic Oligonucleotide
tcgtag

SEQ ID NO: 34 - DNA, 6 bp, Artificial Sequence
  Synthetic Oligonucleotide
tcgtgg

SEQ ID NO: 35 - DNA, 6 bp, Artificial Sequence
  Synthetic Oligonucleotide
cgtcgt

SEQ ID NO: 36 - DNA, 20 bp, Artificial Sequence
  Synthetic Oligonucleotide
tccatgtcggtcctgagtct

SEQ ID NO: 37 - DNA, 20 bp, Artificial Sequence
  Synthetic Oligonucleotide
tccatgccggtcctgagtct

SEQ ID NO: 38 - DNA, 20 bp, Artificial Sequence
  Synthetic Oligonucleotide
tccatgacggtcctgagtct

SEQ ID NO: 39 - DNA, 20 bp, Artificial Sequence
  Synthetic Oligonucleotide
tccatgtcgatcctgagtct

SEQ ID NO: 40 - DNA, 20 bp, Artificial Sequence
  Synthetic Oligonucleotide
tccatgtcgctcctgagtct

SEQ ID NO: 41 - DNA, 20 bp, Artificial Sequence
  Synthetic Oligonucleotide
tccatgtcgttcctgagtct

SEQ ID NO: 42 - DNA, 20 bp, Artificial Sequence
  Synthetic Oligonucleotide
tccatgacgttcctgagtct

SEQ ID NO: 43 - DNA, 20 bp, Artificial Sequence
  Synthetic Oligonucleotide
tccataacgttcctgagtct

SEQ ID NO: 44 - DNA, 20 bp, Artificial Sequence
  Synthetic Oligonucleotide
tccatgacgtccctgagtct

SEQ ID NO: 45 - DNA, 20 bp, Artificial Sequence
  Synthetic Oligonucleotide
tccatcacgtgcctgagtct

SEQ ID NO: 46 - DNA, 20 bp, Artificial Sequence
  Synthetic Oligonucleotide
tccatgctggtcctgagtct

SEQ ID NO: 47 - DNA, 20 bp, Artificial Sequence
  Synthetic Oligonucleotide
  modified_base (8)...(8): m5c
tccatgtcggtcctgagtct

SEQ ID NO: 48 - DNA, 39 bp, Artificial Sequence
  Synthetic Oligonucleotide
ccgcttcctccagatgagctcatgggtttctccaccaag

SEQ ID NO: 49 - DNA, 39 bp, Artificial Sequence
  Synthetic Oligonucleotide
cttggtggagaaacccatgagctcatctggaggaagcgg

SEQ ID NO: 50 - DNA, 20 bp, Artificial Sequence
  Synthetic Oligonucleotide
ccccaaagggatgagaagtt

SEQ ID NO: 51 - DNA, 20 bp, Artificial Sequence
  Synthetic Oligonucleotide
agatagcaaatcggctgacg

SEQ ID NO: 52 - DNA, 20 bp, Artificial Sequence
  Synthetic Oligonucleotide
ggttcacgtgctcatggctg

SEQ ID NO: 53 - DNA, 20 bp, Artificial Sequence
  Synthetic Oligonucleotide
tccatgattttcctgatttt

SEQ ID NO: 54 - DNA, 24 bp, Artificial Sequence
  Synthetic Oligonucleotide
ttgtttttttgtttttttgttttt

SEQ ID NO: 55 - DNA, 22 bp, Artificial Sequence
  Synthetic Oligonucleotide
ttttttttgtttttttgttttt

SEQ ID NO: 56 - DNA, 24 bp, Artificial Sequence
  Synthetic Oligonucleotide
tgctgcttttgtgcttttgtgctt

SEQ ID NO: 57 - DNA, 22 bp, Artificial Sequence
  Synthetic Oligonucleotide
tgctgcttgtgcttttgtgctt

SEQ ID NO: 58 - DNA, 23 bp, Artificial Sequence
  Synthetic Oligonucleotide
gcattcatcaggcgggcaagaat

SEQ ID NO: 59 - DNA, 23 bp, Artificial Sequence
  Synthetic Oligonucleotide
taccgagcttcgacgagatttca

SEQ ID NO: 60 - DNA, 15 bp, Artificial Sequence
  Synthetic Oligonucleotide
gcatgacgttgagct

SEQ ID NO: 61 - DNA, 15 bp, Artificial Sequence
  Synthetic Oligonucleotide
cacgttgaggggcat

SEQ ID NO: 62 - DNA, 15 bp, Artificial Sequence
  Synthetic Oligonucleotide
ctgctgagactggag

SEQ ID NO: 63 - DNA, 20 bp, Artificial Sequence
  Synthetic Oligonucleotide
tccatgacgttcctgacgtt

SEQ ID NO: 64 - DNA, 17 bp, Artificial Sequence
  Synthetic Oligonucleotide
gcatgagcttgagctga

SEQ ID NO: 65 - DNA, 12 bp, Artificial Sequence
  Synthetic Oligonucleotide
tcagcgtgcgcc

SEQ ID NO: 66 - DNA, 17 bp, Artificial Sequence
  Synthetic Oligonucleotide
atgacgttcctgacgtt

SEQ ID NO: 67 - DNA, 20 bp, Artificial Sequence
  Synthetic Oligonucleotide
ttttggggttttggggtttt

SEQ ID NO: 68 - DNA, 20 bp, Artificial Sequence
  Synthetic Oligonucleotide
tctaggctttttaggcttcc

SEQ ID NO: 69 - DNA, 20 bp, Artificial Sequence
  Synthetic Oligonucleotide
tgcattttttaggccaccat

SEQ ID NO: 70 - DNA, 22 bp, Artificial Sequence
  Synthetic Oligonucleotide
tctcccagcgtgcgtgcgccat

SEQ ID NO: 71 - DNA, 17 bp, Artificial Sequence
  Synthetic Oligonucleotide
tctcccagcgggcgcat

SEQ ID NO: 72 - DNA, 18 bp, Artificial Sequence
  Synthetic Oligonucleotide
tctcccagcgagcgccat

SEQ ID NO: 73 - DNA, 18 bp, Artificial Sequence
  Synthetic Oligonucleotide
tctcccagcgcgcgccat

SEQ ID NO: 74 - DNA, 19 bp, Artificial Sequence
  Synthetic Oligonucleotide
ggggtgacgttcagggggg

SEQ ID NO: 75 - DNA, 24 bp, Artificial Sequence
  Synthetic Oligonucleotide
ggggtccagcgtgcgccatggggg

SEQ ID NO: 76 - DNA, 19 bp, Artificial Sequence
  Synthetic Oligonucleotide
ggggtgtcgttcagggggg

SEQ ID NO: 77 - DNA, 20 bp, Artificial Sequence
  Synthetic Oligonucleotide
tccatgtcgttcctgtcgtt

SEQ ID NO: 78 - DNA, 20 bp, Artificial Sequence
  Synthetic Oligonucleotide
tccatagcgttcctagcgtt

SEQ ID NO: 79 - DNA, 21 bp, Artificial Sequence
  Synthetic Oligonucleotide
tcgtcgctgtctccgcttctt

SEQ ID NO: 80 - DNA, 15 bp, Artificial Sequence
  Synthetic Oligonucleotide
gcatgacgttgagct

SEQ ID NO: 81 - DNA, 20 bp, Artificial Sequence
  Synthetic Oligonucleotide
tctcccagcgtgcgccatat

SEQ ID NO: 82 - DNA, 20 bp, Artificial Sequence
  Synthetic Oligonucleotide
  modified_base (8)...(8): m5c
  modified_base (17)...(17): m5c
tccatgacgttcctgacgtt

SEQ ID NO: 83 - DNA, 15 bp, Artificial Sequence
  Synthetic Oligonucleotide
  modified_base (7)...(7): m5c
gcatgacgttgagct

SEQ ID NO: 84 - DNA, 16 bp, Artificial Sequence
  Synthetic Oligonucleotide
tccagcgtgcgccata

SEQ ID NO: 85 - DNA, 18 bp, Artificial Sequence
  Synthetic Oligonucleotide
tctcccagcgtgcgccat

SEQ ID NO: 86 - DNA, 15 bp, Artificial Sequence
  Synthetic Oligonucleotide
gcatgtcgttgagct

SEQ ID NO: 87 - DNA, 19 bp, Artificial Sequence
  Synthetic Oligonucleotide
tcctgacgttcctgacgtt

SEQ ID NO: 88 - DNA, 15 bp, Artificial Sequence
  Synthetic Oligonucleotide
gcatgatgttgagct

SEQ ID NO: 89 - DNA, 15 bp, Artificial Sequence
  Synthetic Oligonucleotide
gcatttcgaggagct

SEQ ID NO: 90 - DNA, 15 bp, Artificial Sequence
  Synthetic Oligonucleotide
gcatgtagctgagct

SEQ ID NO: 91 - DNA, 20 bp, Artificial Sequence
  Synthetic Oligonucleotide
tccaggacgttcctagttct

SEQ ID NO: 92 - DNA, 20 bp, Artificial Sequence
  Synthetic Oligonucleotide
tccaggagcttcctagttct

SEQ ID NO: 93 - DNA, 20 bp, Artificial Sequence
  Synthetic Oligonucleotide
tccaggatgttcctagttct

SEQ ID NO: 94 - DNA, 20 bp, Artificial Sequence
  Synthetic Oligonucleotide
tccagtctaggcctagttct

SEQ ID NO: 95 - DNA, 20 bp, Artificial Sequence
  Synthetic Oligonucleotide
tccagttcgagcctagttct

SEQ ID NO: 96 - DNA, 15 bp, Artificial Sequence
  Synthetic Oligonucleotide
gcatggcgttgagct

SEQ ID NO: 97 - DNA, 15 bp, Artificial Sequence
  Synthetic Oligonucleotide
gcatagcgttgagct

SEQ ID NO: 98 - DNA, 15 bp, Artificial Sequence
  Synthetic Oligonucleotide
gcattgcgttgagct

SEQ ID NO: 99 - DNA, 15 bp, Artificial Sequence
  Synthetic Oligonucleotide
gcttgcgttgcgttt

SEQ ID NO: 100 - DNA, 21 bp, Artificial Sequence
  Synthetic Oligonucleotide
tctcccagcgttgcgccatat

SEQ ID NO: 101 - DNA, 20 bp, Artificial Sequence
  Synthetic Oligonucleotide
tctcccagcgtgcgttatat

SEQ ID NO: 102 - DNA, 20 bp, Artificial Sequence
  Synthetic Oligonucleotide
tctccctgcgtgcgccatat

SEQ ID NO: 103 - DNA, 20 bp, Artificial Sequence
  Synthetic Oligonucleotide
tctgcgtgcgtgcgccatat

SEQ ID NO: 104 - DNA, 20 bp, Artificial Sequence
  Synthetic Oligonucleotide
tctcctagcgtgcgccatat

SEQ ID NO: 105 - DNA, 20 bp, Artificial Sequence
  Synthetic Oligonucleotide
tctcccagcgtgcgcctttt

SEQ ID NO: 106 - DNA, 13 bp, Artificial Sequence
  Synthetic Oligonucleotide
gctandcghhagc

SEQ ID NO: 107 - DNA, 13 bp, Artificial Sequence
  Synthetic Oligonucleotide
tcctgacgttccc

SEQ ID NO: 108 - DNA, 13 bp, Artificial Sequence
  Synthetic Oligonucleotide
ggaagacgttaga

SEQ ID NO: 109 - DNA, 13 bp, Artificial Sequence
  Synthetic Oligonucleotide
tcctgacgttaga

SEQ ID NO: 110 - DNA, 27 bp, Artificial Sequence
  Synthetic Oligonucleotide
tcagaccagctggtcgggtgttcctga

SEQ ID NO: 111 - DNA, 27 bp, Artificial Sequence
  Synthetic Oligonucleotide
tcaggaacacccgaccagctggtctga

SEQ ID NO: 112 - DNA, 13 bp, Artificial Sequence
  Synthetic Oligonucleotide
gctagtcgatagc

SEQ ID NO: 113 - DNA, 13 bp, Artificial Sequence
  Synthetic Oligonucleotide
gctagtcgctagc

SEQ ID NO: 114 - DNA, 14 bp, Artificial Sequence
  Synthetic Oligonucleotide
gcttgacgtctagc

SEQ ID NO: 115 - DNA, 14 bp, Artificial Sequence
  Synthetic Oligonucleotide
gcttgacgtttagc

SEQ ID NO: 116 - DNA, 14 bp, Artificial Sequence
  Synthetic Oligonucleotide
gcttgacgtcaagc

SEQ ID NO: 117 - DNA, 14 bp, Artificial Sequence
  Synthetic Oligonucleotide
gctagacgtttagc

SEQ ID NO: 118 - DNA, 20 bp, Artificial Sequence
  Synthetic Oligonucleotide
tccatgacattcctgatgct

SEQ ID NO: 119 - DNA, 14 bp, Artificial Sequence
  Synthetic Oligonucleotide
gctagacgtctagc

SEQ ID NO: 120 - DNA, 19 bp, Artificial Sequence
  Synthetic Oligonucleotide
ggctatgtcgttcctagcc

SEQ ID NO: 121 - DNA, 19 bp, Artificial Sequence
  Synthetic Oligonucleotide
ggctatgtcgatcctagcc

SEQ ID NO: 122 - DNA, 21 bp, Artificial Sequence
  Synthetic Oligonucleotide
ctcatgggtttctccaccaag

SEQ ID NO: 123 - DNA, 21 bp, Artificial Sequence
  Synthetic Oligonucleotide
cttggtggagaaacccatgag

SEQ ID NO: 124 - DNA, 20 bp, Artificial Sequence
  Synthetic Oligonucleotide
tccatgacgttcctagttct

SEQ ID NO: 125 - DNA, 24 bp, Artificial Sequence
  Synthetic Oligonucleotide
ccgcttcctccagatgagctcatg

SEQ ID NO: 126 - DNA, 24 bp, Artificial Sequence
  Synthetic Oligonucleotide
catgagctcatctggaggaagcgg

SEQ ID NO: 127 - DNA, 24 bp, Artificial Sequence
  Synthetic Oligonucleotide
ccagatgagctcatgggtttctcc

SEQ ID NO: 128 - DNA, 24 bp, Artificial Sequence
  Synthetic Oligonucleotide
ggagaaacccatgagctcatctgg

SEQ ID NO: 129 - DNA, 20 bp, Artificial Sequence
  Synthetic Oligonucleotide
agcatcaggaacgacatgga

SEQ ID NO: 130 - DNA, 20 bp, Artificial Sequence
  Synthetic Oligonucleotide
tccatgacgttcctgacgtt

SEQ ID NO: 131 - DNA, 19 bp, Artificial Sequence
  Synthetic Oligonucleotide
gcgcgcgcgcgcgcgcgcg

SEQ ID NO: 132 - DNA, 20 bp, Artificial Sequence
  Synthetic Oligonucleotide
ccggccggccggccggccgg

SEQ ID NO: 133 - DNA, 43 bp, Artificial Sequence
  Synthetic Oligonucleotide
ttccaatcagccccacccgctctggccccaccctcaccctcca

SEQ ID NO: 134 - DNA, 43 bp, Artificial Sequence
  Synthetic Oligonucleotide
tggagggtgagggtggggccagagcgggtggggctgattggaa

SEQ ID NO: 135 - DNA, 27 bp, Artificial Sequence
  Synthetic Oligonucleotide
tcaaatgtgggattttcccatgagtct

SEQ ID NO: 136 - DNA, 27 bp, Artificial Sequence
  Synthetic Oligonucleotide
agactcatgggaaaatcccacatttga

SEQ ID NO: 137 - DNA, 27 bp, Artificial Sequence
  Synthetic Oligonucleotide
tgccaagtgctgagtcactaataaaga

SEQ ID NO: 138 - DNA, 27 bp, Artificial Sequence
  Synthetic Oligonucleotide
tctttattagtgactcagcacttggca

SEQ ID NO: 139 - DNA, 31 bp, Artificial Sequence
  Synthetic Oligonucleotide
tgcaggaagtccgggttttccccaacccccc

SEQ ID NO: 140 - DNA, 31 bp, Artificial Sequence
  Synthetic Oligonucleotide
ggggggttggggaaaacccggacttcctgca

SEQ ID NO: 141 - DNA, 38 bp, Artificial Sequence
  Synthetic Oligonucleotide
ggggactttccgctggggactttccagggggactttcc

SEQ ID NO: 142 - DNA, 45 bp, Artificial Sequence
  Synthetic Oligonucleotide
tccatgacgttcctctccatgacgttcctctccatgacgttcctc

SEQ ID NO: 143 - DNA, 45 bp, Artificial Sequence
  Synthetic Oligonucleotide
gaggaacgtcatggagaggaacgtcatggagaggaacgtcatgga

SEQ ID NO: 144 - DNA, 20 bp, Artificial Sequence
  Synthetic Oligonucleotide
ataatagagcttcaagcaag

SEQ ID NO: 145 - DNA, 20 bp, Artificial Sequence
  Synthetic Oligonucleotide
tccatgacgttcctgacgtt

SEQ ID NO: 146 - DNA, 20 bp, Artificial Sequence
  Synthetic Oligonucleotide
tccatgacgttcctgacgtt

SEQ ID NO: 147 - DNA, 20 bp, Artificial Sequence
  Synthetic Oligonucleotide
tccaggactttcctcaggtt

SEQ ID NO: 148 - DNA, 45 bp, Artificial Sequence
  Synthetic Oligonucleotide
tcttgcgatgctaaaggacgtcacattgcacaatcttaataaggt

SEQ ID NO: 149 - DNA, 45 bp, Artificial Sequence
  Synthetic Oligonucleotide
accttattaagattgtgcaatgtgacgtcctttagcatcgcaaga

SEQ ID NO: 150 - DNA, 28 bp, Artificial Sequence
  Synthetic Oligonucleotide
tcctgacgttcctggcggtcctgtcgct

SEQ ID NO: 151 - DNA, 19 bp, Artificial Sequence
  Synthetic Oligonucleotide
tcctgtcgctcctgtcgct

SEQ ID NO: 152 - DNA, 15 bp, Artificial Sequence
  Synthetic Oligonucleotide
tcctgacgttgaagt

SEQ ID NO: 153 - DNA, 15 bp, Artificial Sequence
  Synthetic Oligonucleotide
tcctgtcgttgaagt

SEQ ID NO: 154 - DNA, 15 bp, Artificial Sequence
  Synthetic Oligonucleotide
tcctggcgttgaagt

SEQ ID NO: 155 - DNA, 15 bp, Artificial Sequence
  Synthetic Oligonucleotide
tcctgccgttgaagt

SEQ ID NO: 156 - DNA, 15 bp, Artificial Sequence
  Synthetic Oligonucleotide
tccttacgttgaagt

SEQ ID NO: 157 - DNA, 15 bp, Artificial Sequence
  Synthetic Oligonucleotide
tcctaacgttgaagt

SEQ ID NO: 158 - DNA, 15 bp, Artificial Sequence
  Synthetic Oligonucleotide
tcctcacgttgaagt

SEQ ID NO: 159 - DNA, 15 bp, Artificial Sequence
  Synthetic Oligonucleotide
tcctgacgatgaagt

SEQ ID NO: 160 - DNA, 15 bp, Artificial Sequence
  Synthetic Oligonucleotide
tcctgacgctgaagt

SEQ ID NO: 161 - DNA, 15 bp, Artificial Sequence
  Synthetic Oligonucleotide
tcctgacggtgaagt

SEQ ID NO: 162 - DNA, 15 bp, Artificial Sequence
  Synthetic Oligonucleotide
tcctgacgtagaagt

SEQ ID NO: 163 - DNA, 15 bp, Artificial Sequence
  Synthetic Oligonucleotide
tcctgacgtcgaagt

SEQ ID NO: 164 - DNA, 15 bp, Artificial Sequence
  Synthetic Oligonucleotide
tcctgacgtggaagt

SEQ ID NO: 165 - DNA, 15 bp, Artificial Sequence
  Synthetic Oligonucleotide
tcctgagcttgaagt

SEQ ID NO: 166 - DNA, 15 bp, Artificial Sequence
  Synthetic Oligonucleotide
gggggacgttggggg

SEQ ID NO: 167 - DNA, 15 bp, Artificial Sequence
  Synthetic Oligonucleotide
tcctgacgttccttc

SEQ ID NO: 168 - DNA, 22 bp, Artificial Sequence
  Synthetic Oligonucleotide
tctcccagcgagcgagcgccat

SEQ ID NO: 169 - DNA, 32 bp, Artificial Sequence
  Synthetic Oligonucleotide
tcctgacgttcccctggcggtcccctgtcgct

SEQ ID NO: 170 - DNA, 28 bp, Artificial Sequence
  Synthetic Oligonucleotide
tcctgtcgctcctgtcgctcctgtcgct

SEQ ID NO: 171 - DNA, 15 bp, Artificial Sequence
  Synthetic Oligonucleotide
tcctggcggggaagt

SEQ ID NO: 172 - DNA, 15 bp, Artificial Sequence
  Synthetic Oligonucleotide
  modified_base (7)...(7): m5c
tcctgacgttgaagt

SEQ ID NO: 173 - DNA, 15 bp, Artificial Sequence
  Synthetic Oligonucleotide
  modified_base (3)...(3): m5c
tcctgacgttgaagt

SEQ ID NO: 174 - DNA, 15 bp, Artificial Sequence
  Synthetic Oligonucleotide
tcctagcgttgaagt

SEQ ID NO: 175 - DNA, 15 bp, Artificial Sequence
  Synthetic Oligonucleotide
tccagacgttgaagt

SEQ ID NO: 176 - DNA, 15 bp, Artificial Sequence
  Synthetic Oligonucleotide
tcctgacggggaagt

SEQ ID NO: 177 - DNA, 15 bp, Artificial Sequence
  Synthetic Oligonucleotide
tcctggcggtgaagt

SEQ ID NO: 178 - DNA, 27 bp, Artificial Sequence
  Synthetic Oligonucleotide
ggctccggggagggaatttttgtctat

SEQ ID NO: 179 - DNA, 27 bp, Artificial Sequence
  Synthetic Oligonucleotide
atagacaaaaattccctccccggagcc

SEQ ID NO: 180 - DNA, 21 bp, Artificial Sequence
  Synthetic Oligonucleotide
tccatgagcttccttgagtct

SEQ ID NO: 181 - DNA, 21 bp, Artificial Sequence
  Synthetic Oligonucleotide
tcgtcgctgtctccgcttctt

SEQ ID NO: 182 - DNA, 21 bp, Artificial Sequence
  Synthetic Oligonucleotide
tcgtcgctgtctccgcttctt

SEQ ID NO: 183 - DNA, 23 bp, Artificial Sequence
  Synthetic Oligonucleotide
tcgagacattgcacaatcatctg

SEQ ID NO: 184 - DNA, 20 bp, Artificial Sequence
  Synthetic Oligonucleotide
cagattgtgcaatgtctcga

SEQ ID NO: 185 - DNA, 20 bp, Artificial Sequence
  Synthetic Oligonucleotide
tccatgtcgttcctgatgcg

SEQ ID NO: 186 - DNA, 20 bp, Artificial Sequence
  Synthetic Oligonucleotide
gcgatgtcgttcctgatgct

SEQ ID NO: 187 - DNA, 20 bp, Artificial Sequence
  Synthetic Oligonucleotide
gcgatgtcgttcctgatgcg

SEQ ID NO: 188 - DNA, 20 bp, Artificial Sequence
  Synthetic Oligonucleotide
tccatgtcgttccgcgcgcg

SEQ ID NO: 189 - DNA, 20 bp, Artificial Sequence
  Synthetic Oligonucleotide
tccatgtcgttcctgccgct

SEQ ID NO: 190 - DNA, 20 bp, Artificial Sequence
  Synthetic Oligonucleotide
tccatgtcgttcctgtagct

SEQ ID NO: 191 - DNA, 20 bp, Artificial Sequence
  Synthetic Oligonucleotide
gcggcgggcggcgcgcgccc

SEQ ID NO: 192 - DNA, 21 bp, Artificial Sequence
  Synthetic Oligonucleotide
atcaggaacgtcatgggaagc

SEQ ID NO: 193 - DNA, 20 bp, Artificial Sequence
  Synthetic Oligonucleotide
tccatgagcttcctgagtct

SEQ ID NO: 194 - DNA, 8 bp, Artificial Sequence
  Synthetic Oligonucleotide
tcaacgtt

SEQ ID NO: 195 - DNA, 8 bp, Artificial Sequence
  Synthetic Oligonucleotide
tcaagctt

SEQ ID NO: 196 - DNA, 19 bp, Artificial Sequence
  Synthetic Oligonucleotide
tcctgtcgttcctgtcgtt

SEQ ID NO: 197 - DNA, 20 bp, Artificial Sequence
  Synthetic Oligonucleotide
tccatgtcgtttttgtcgtt

SEQ ID NO: 198 - DNA, 20 bp, Artificial Sequence
  Synthetic Oligonucleotide
tcctgtcgttccttgtcgtt

SEQ ID NO: 199 - DNA, 20 bp, Artificial Sequence
  Synthetic Oligonucleotide
tccttgtcgttcctgtcgtt

SEQ ID NO: 200 - DNA, 29 bp, Artificial Sequence
  Synthetic Oligonucleotide
  misc_feature (1)...(1): biotinylated
tccattccatgacgttcctgatgcttcca

SEQ ID NO: 201 - DNA, 20 bp, Artificial Sequence
  Synthetic Oligonucleotide
tcctgtcgttttttgtcgtt

SEQ ID NO: 202 - DNA, 21 bp, Artificial Sequence
  Synthetic Oligonucleotide
tcgtcgctgtctccgcttctt

SEQ ID NO: 203 - DNA, 21 bp, Artificial Sequence
  Synthetic Oligonucleotide
tcgtcgctgtctgcccttctt

SEQ ID NO: 204 - DNA, 21 bp, Artificial Sequence
  Synthetic Oligonucleotide
tcgtcgctgttgtcgtttctt

SEQ ID NO: 205 - DNA, 30 bp, Artificial Sequence
  Synthetic Oligonucleotide
tcctgtcgttcctgtcgttggaacgacagg

SEQ ID NO: 206 - DNA, 40 bp, Artificial Sequence
  Synthetic Oligonucleotide
tcctgtcgttcctgtcgtttcaacgtcaggaacgacagga

SEQ ID NO: 207 - DNA, 21 bp, Artificial Sequence
  Synthetic Oligonucleotide
ggggtctgtcgttttgggggg

SEQ ID NO: 208 - DNA, 21 bp, Artificial Sequence
  Synthetic Oligonucleotide
ggggtctgtgcttttgggggg

SEQ ID NO: 209 - DNA, 15 bp, Artificial Sequence
  Synthetic Oligonucleotide
tccggccgttgaagt

SEQ ID NO: 210 - DNA, 15 bp, Artificial Sequence
  Synthetic Oligonucleotide
tccggacggtgaagt

SEQ ID NO: 211 - DNA, 15 bp, Artificial Sequence
  Synthetic Oligonucleotide
tcccgccgttgaagt

SEQ ID NO: 212 - DNA, 15 bp, Artificial Sequence
  Synthetic Oligonucleotide
tccagacggtgaagt

SEQ ID NO: 213 - DNA, 15 bp, Artificial Sequence
  Synthetic Oligonucleotide
tcccgacggtgaagt

SEQ ID NO: 214 - DNA, 15 bp, Artificial Sequence
  Synthetic Oligonucleotide
tccagagcttgaagt

SEQ ID NO: 215 - DNA, 20 bp, Artificial Sequence
  Synthetic Oligonucleotide
  modified_base (8)...(8): m5c
  modified_base (7)...(17): m5c
tccatgtcgttcctgtcgtt

SEQ ID NO: 216 - DNA, 20 bp, Artificial Sequence
  Synthetic Oligonucleotide
ggggttgacgttttgggggg

SEQ ID NO: 217 - DNA, 20 bp, Artificial Sequence
  Synthetic Oligonucleotide
tccaggacttctctcaggtt

SEQ ID NO: 218 - DNA, 20 bp, Artificial Sequence
  Synthetic Oligonucleotide
tttttttttttttttttttt

SEQ ID NO: 219 - DNA, 20 bp, Artificial Sequence
  Synthetic Oligonucleotide
tccatgccgttcctgccgtt

SEQ ID NO: 220 - DNA, 20 bp, Artificial Sequence
  Synthetic Oligonucleotide
tccatggcgggcctggcggg

SEQ ID NO: 221 - DNA, 20 bp, Artificial Sequence
  Synthetic Oligonucleotide
tccatgacgttcctgccgtt

SEQ ID NO: 222 - DNA, 20 bp, Artificial Sequence
  Synthetic Oligonucleotide
tccatgacgttcctggcggg

SEQ ID NO: 223 - DNA, 20 bp, Artificial Sequence
  Synthetic Oligonucleotide
tccatgacgttcctgcgttt

SEQ ID NO: 224 - DNA, 20 bp, Artificial Sequence
  Synthetic Oligonucleotide
tccatgacggtcctgacggt

SEQ ID NO: 225 - DNA, 20 bp, Artificial Sequence
  Synthetic Oligonucleotide
tccatgcgtgcgtgcgtttt

SEQ ID NO: 226 - DNA, 20 bp, Artificial Sequence
  Synthetic Oligonucleotide
tccatgcgttgcgttgcgtt

SEQ ID NO: 227 - DNA, 30 bp, Artificial Sequence
  Synthetic Oligonucleotide
  misc_feature (1)...(1): biotinylated
tccattccattctaggcctgagtcttccat

SEQ ID NO: 228 - DNA, 20 bp, Artificial Sequence
  Synthetic Oligonucleotide
tccatagcgttcctagcgtt

SEQ ID NO: 229 - DNA, 20 bp, Artificial Sequence
  Synthetic Oligonucleotide
tccatgtcgttcctgtcgtt

SEQ ID NO: 230 - DNA, 20 bp, Artificial Sequence
  Synthetic Oligonucleotide
tccatagcgatcctagcgat

SEQ ID NO: 231 - DNA, 20 bp, Artificial Sequence
  Synthetic Oligonucleotide
tccattgcgttccttgcgtt

SEQ ID NO: 232 - DNA, 20 bp, Artificial Sequence
  Synthetic Oligonucleotide
tccatagcggtcctagcggt

SEQ ID NO: 233 - DNA, 29 bp, Artificial Sequence
  Synthetic Oligonucleotide
tccatgattttcctgcagttcctgatttt

SEQ ID NO: 234 - DNA, 29 bp, Artificial Sequence
  Synthetic Oligonucleotide
tccatgacgttcctgcagttcctgacgtt

SEQ ID NO: 235 - DNA, 20 bp, Artificial Sequence
  Synthetic Oligonucleotide
ggcggcggcggcggcggcgg

SEQ ID NO: 236 - DNA, 20 bp, Artificial Sequence
  Synthetic Oligonucleotide
tccacgacgttttcgacgtt

SEQ ID NO: 237 - DNA, 20 bp, Artificial Sequence
  Synthetic Oligonucleotide
tcgtcgttgtcgttgtcgtt

SEQ ID NO: 238 - DNA, 24 bp, Artificial Sequence
  Synthetic Oligonucleotide
tcgtcgttttgtcgttttgtcgtt

SEQ ID NO: 239 - DNA, 22 bp, Artificial Sequence
  Synthetic Oligonucleotide
tcgtcgttgtcgttttgtcgtt

SEQ ID NO: 240 - DNA, 21 bp, Artificial Sequence
  Synthetic Oligonucleotide
gcgtgcgttgtcgttgtcgtt

SEQ ID NO: 241 - DNA, 19 bp, Artificial Sequence
  Synthetic Oligonucleotide
  modified_base (2)...(2): m5c AME/KEY: modified_bse
  (6)...(6): m5c
  modified_base (10)...(10): m5c
  modified_base (15)...(0): m5c
ccggccggccgggccccgg

SEQ ID NO: 242 - DNA, 20 bp, Artificial Sequence
  Synthetic Oligonucleotide
gcggcgggcggcgcgcgccc

SEQ ID NO: 243 - DNA, 20 bp, Artificial Sequence
  Synthetic Oligonucleotide
  modified_base (3)...(3): I AME/KEY: modified_bse
  (8)...(8): I
  modified_base (14)...(14): I
agncccgngaacgnattcac

SEQ ID NO: 244 - DNA, 21 bp, Artificial Sequence
  Synthetic Oligonucleotide
tgtcgtttgtcgtttgtcgtt

SEQ ID NO: 245 - DNA, 25 bp, Artificial Sequence
  Synthetic Oligonucleotide
tgtcgttgtcgttgtcgttgtcgtt

SEQ ID NO: 246 - DNA, 14 bp, Artificial Sequence
  Synthetic Oligonucleotide
tcgtcgtcgtcgtt

SEQ ID NO: 247 - DNA, 13 bp, Artificial Sequence
  Synthetic Oligonucleotide
tgtcgttgtcgtt

SEQ ID NO: 248 - DNA, 20 bp, Artificial Sequence
  Synthetic Oligonucleotide
cccccccccccccccccccc

SEQ ID NO: 249 - DNA, 20 bp, Artificial Sequence
  Synthetic Oligonucleotide
tctagcgtttttagcgttcc

SEQ ID NO: 250 - DNA, 20 bp, Artificial Sequence
  Synthetic Oligonucleotide
tgcatcccccaggccaccat

SEQ ID NO: 251 - DNA, 23 bp, Artificial Sequence
  Synthetic Oligonucleotide
tcgtcgtcgtcgtcgtcgtcgtt

SEQ ID NO: 252 - DNA, 20 bp, Artificial Sequence
  Synthetic Oligonucleotide
tcgtcgttgtcgttgtcgtt

SEQ ID NO: 253 - DNA, 24 bp, Artificial Sequence
  Synthetic Oligonucleotide
tcgtcgttttgtcgttttgtcgtt

SEQ ID NO: 254 - DNA, 22 bp, Artificial Sequence
  Synthetic Oligonucleotide
tcgtcgttgtcgttttgtcgtt

SEQ ID NO: 255 - DNA, 39 bp, Artificial Sequence
  Synthetic Oligonucleotide
ggggagggaggaacttcttaaaattcccccagaatgttt

SEQ ID NO: 256 - DNA, 39 bp, Artificial Sequence
  Synthetic Oligonucleotide
aaacattctgggggaattttaagaagttcctccctcccc

SEQ ID NO: 257 - DNA, 33 bp, Artificial Sequence
  Synthetic Oligonucleotide
atgtttacttcttaaaattcccccagaatgttt

SEQ ID NO: 258 - DNA, 33 bp, Artificial Sequence
  Synthetic Oligonucleotide
aaacattctgggggaattttaagaagtaaacat

SEQ ID NO: 259 - DNA, 33 bp, Artificial Sequence
  Synthetic Oligonucleotide
atgtttactagacaaaattcccccagaatgttt

SEQ ID NO: 260 - DNA, 33 bp, Artificial Sequence
  Synthetic Oligonucleotide
aaacattctgggggaattttgtctagtaaacat

SEQ ID NO: 261 - DNA, 20 bp, Artificial Sequence
  Synthetic Oligonucleotide
aaaattgacgttttaaaaaa

SEQ ID NO: 262 - DNA, 20 bp, Artificial Sequence
  Synthetic Oligonucleotide
ccccttgacgttttcccccc

SEQ ID NO: 263 - DNA, 20 bp, Artificial Sequence
  Synthetic Oligonucleotide
ttttcgttgtttttgtcgtt

SEQ ID NO: 264 - DNA, 14 bp, Artificial Sequence
  Synthetic Oligonucleotide
ctgcagcctgggac

SEQ ID NO: 265 - DNA, 25 bp, Artificial Sequence
  Synthetic Oligonucleotide
acccgtcgtaattatagtaaaaccc

SEQ ID NO: 266 - DNA, 21 bp, Artificial Sequence
  Synthetic Oligonucleotide
ggtacctgtggggacattgtg

SEQ ID NO: 267 - DNA, 18 bp, Artificial Sequence
  Synthetic Oligonucleotide
agcaccgaacgtgagagg

SEQ ID NO: 268 - DNA, 20 bp, Artificial Sequence
  Synthetic Oligonucleotide
tccatgccgttcctgccgtt

SEQ ID NO: 269 - DNA, 20 bp, Artificial Sequence
  Synthetic Oligonucleotide
tccatgacggtcctgacggt

SEQ ID NO: 270 - DNA, 20 bp, Artificial Sequence
  Synthetic Oligonucleotide
tccatgccggtcctgccggt

SEQ ID NO: 271 - DNA, 20 bp, Artificial Sequence
  Synthetic Oligonucleotide
tccatgcgcgtcctgcgcgt

SEQ ID NO: 272 - DNA, 24 bp, Artificial Sequence
  Synthetic Oligonucleotide
ctggtctttctggtttttttctgg

SEQ ID NO: 273 - DNA, 20 bp, Artificial Sequence
  Synthetic Oligonucleotide
tcaggggtggggggaacctt

SEQ ID NO: 274 - DNA, 20 bp, Artificial Sequence
  Synthetic Oligonucleotide
  modified_base (8)...(8): m5c
tccatgacgttcctagttct

SEQ ID NO: 275 - DNA, 20 bp, Artificial Sequence
  Synthetic Oligonucleotide
tccatgatgttcctagttct

SEQ ID NO: 276 - DNA, 26 bp, Artificial Sequence
  Synthetic Oligonucleotide
cccgaagtcatttcctcttaacctgg

SEQ ID NO: 277 - DNA, 26 bp, Artificial Sequence
  Synthetic Oligonucleotide
ccaggttaagaggaaatgacttcggg

SEQ ID NO: 278 - DNA, 15 bp, Artificial Sequence
  Synthetic Oligonucleotide
  modified_base (7)...(7): m5c
tcctggcggggaagt

SEQ ID NO: 279 - DNA, 20 bp, Artificial Sequence
  Synthetic Oligonucleotide
  modified_base (2)...(2): m5c
  modified_base (5)...(5): m5c
  modified_base (9)...(9): m5c
  modified_base (12)...(12): m5c
  modified_base (14)...(14): m5c
  modified_base (16)...(16): m5c
gcggcgggcggcgcgcgccc

SEQ ID NO: 280 - DNA, 20 bp, Artificial Sequence
  Synthetic Oligonucleotide
tccatgtgcttcctgatgct

SEQ ID NO: 281 - DNA, 20 bp, Artificial Sequence
  Synthetic Oligonucleotide
tccatgtccttcctgatgct

SEQ ID NO: 282 - DNA, 20 bp, Artificial Sequence
  Synthetic Oligonucleotide
tccatgtcgttcctagttct

SEQ ID NO: 283 - DNA, 20 bp, Artificial Sequence
  Synthetic Oligonucleotide
tccaagtagttcctagttct

SEQ ID NO: 284 - DNA, 20 bp, Artificial Sequence
  Synthetic Oligonucleotide
tccatgtagttcctagttct

SEQ ID NO: 285 - DNA, 20 bp, Artificial Sequence
  Synthetic Oligonucleotide
tcccgcgcgttccgcgcgtt

SEQ ID NO: 286 - DNA, 20 bp, Artificial Sequence
  Synthetic Oligonucleotide
tcctggcggtcctggcggtt

SEQ ID NO: 287 - DNA, 15 bp, Artificial Sequence
  Synthetic Oligonucleotide
tcctggaggggaagt

SEQ ID NO: 288 - DNA, 15 bp, Artificial Sequence
  Synthetic Oligonucleotide
tcctgggggggaagt

SEQ ID NO: 289 - DNA, 15 bp, Artificial Sequence
  Synthetic Oligonucleotide
tcctggtggggaagt

SEQ ID NO: 290 - DNA, 24 bp, Artificial Sequence
  Synthetic Oligonucleotide
tcgtcgttttgtcgttttgtcgtt

SEQ ID NO: 291 - DNA, 24 bp, Artificial Sequence
  Synthetic Oligonucleotide
ctggtctttctggtttttttctgg

SEQ ID NO: 292 - DNA, 20 bp, Artificial Sequence
  Synthetic Oligonucleotide
tccatgacgttcctgacgtt

SEQ ID NO: 293 - DNA, 20 bp, Artificial Sequence
  Synthetic Oligonucleotide
tccaggacttctctcaggtt

SEQ ID NO: 294 - DNA, 24 bp, Artificial Sequence
  Synthetic Oligonucleotide
  modified_base (2)...(2): m5c AME/KEY: modified_bse
  (5)...(5): m5c
  modified_base (13)...(13): m5c
  modified_base (21)...(21): m5c
tcgtcgttttgtcgttttgtcgtt

SEQ ID NO: 295 - DNA, 29 bp, Artificial Sequence
  Synthetic Oligonucleotide
  misc_feature (1)...(1): biotinylated
tcgtcgttttgtcgttttgtcgttttttt

SEQ ID NO: 296 - DNA, 18 bp, Artificial Sequence
  Synthetic Oligonucleotide
gctatgacgttccaaggg

SEQ ID NO: 297 - DNA, 8 bp, Artificial Sequence
  Synthetic Oligonucleotide
tcaacgtt

SEQ ID NO: 298 - DNA, 20 bp, Artificial Sequence
  Synthetic Oligonucleotide
tccaggactttcctcaggtt

SEQ ID NO: 299 - DNA, 20 bp, Artificial Sequence
  Synthetic Oligonucleotide
ctctctgtaggcccgcttgg

SEQ ID NO: 300 - DNA, 20 bp, Artificial Sequence
  Synthetic Oligonucleotide
ctttccgttggacccctggg

SEQ ID NO: 301 - DNA, 20 bp, Artificial Sequence
  Synthetic Oligonucleotide
gtccgggccaggccaaagtc

SEQ ID NO: 302 - DNA, 20 bp, Artificial Sequence
  Synthetic Oligonucleotide
gtgcgcgcgagcccgaaatc

SEQ ID NO: 303 - DNA, 20 bp, Artificial Sequence
  Synthetic Oligonucleotide
  modified_base (8)...(8): I
  modified_base (17)...(17): I
tccatgangttcctgangtt

SEQ ID NO: 304 - DNA, 20 bp, Artificial Sequence
  Synthetic Oligonucleotide
aatagtcgccataacaaaac

SEQ ID NO: 305 - DNA, 20 bp, Artificial Sequence
  Synthetic Oligonucleotide
aatagtcgccatggcggggc

SEQ ID NO: 306 - DNA, 28 bp, Artificial Sequence
  Synthetic Oligonucleotide
  misc_feature (1)...(1): biotinylated
tttttccatgtcgttcctgatgcttttt

SEQ ID NO: 307 - DNA, 20 bp, Artificial Sequence
  Synthetic Oligonucleotide
tcctgtcgttgaagtttttt

SEQ ID NO: 308 - DNA, 24 bp, Artificial Sequence
  Synthetic Oligonucleotide
gctagctttagagctttagagctt

SEQ ID NO: 309 - DNA, 20 bp, Artificial Sequence
  Synthetic Oligonucleotide
tgctgcttcccccccccccc

SEQ ID NO: 310 - DNA, 20 bp, Artificial Sequence
  Synthetic Oligonucleotide
tcgacgttcccccccccccc

SEQ ID NO: 311 - DNA, 20 bp, Artificial Sequence
  Synthetic Oligonucleotide
tcgtcgttcccccccccccc

SEQ ID NO: 312 - DNA, 20 bp, Artificial Sequence
  Synthetic Oligonucleotide
tcgccgttcccccccccccc

SEQ ID NO: 313 - DNA, 20 bp, Artificial Sequence
  Synthetic Oligonucleotide
tcgtcgatcccccccccccc

SEQ ID NO: 314 - DNA, 15 bp, Artificial Sequence
  Synthetic Oligonucleotide
tcctgacgttgaagt

SEQ ID NO: 315 - DNA, 15 bp, Artificial Sequence
  Synthetic Oligonucleotide
tcctgccgttgaagt

SEQ ID NO: 316 - DNA, 15 bp, Artificial Sequence
  Synthetic Oligonucleotide
tcctgacggtgaagt

SEQ ID NO: 317 - DNA, 15 bp, Artificial Sequence
  Synthetic Oligonucleotide
tcctgagcttgaagt

SEQ ID NO: 318 - DNA, 15 bp, Artificial Sequence
  Synthetic Oligonucleotide
tcctggcggggaagt

SEQ ID NO: 319 - DNA, 21 bp, Artificial Sequence
  Synthetic Oligonucleotide
aaaatctgtgcttttaaaaaa

SEQ ID NO: 320 - DNA, 33 bp, Artificial Sequence
  Synthetic Oligonucleotide
gatccagtcacagtgacctggcagaatctggat

SEQ ID NO: 321 - DNA, 33 bp, Artificial Sequence
  Synthetic Oligonucleotide
gatccagattctgccaggtcactgtgactggat

SEQ ID NO: 322 - DNA, 33 bp, Artificial Sequence
  Synthetic Oligonucleotide
gatccagtcacagtgactcagcagaatctggat

SEQ ID NO: 323 - DNA, 33 bp, Artificial Sequence
  Synthetic Oligonucleotide
gatccagattctgctgagtcactgtgactggat

SEQ ID NO: 324 - DNA, 20 bp, Artificial Sequence
  Synthetic Oligonucleotide
  modified_base (16)...(16): m5c
tcgtcgttcccccccccccc

SEQ ID NO: 325 - DNA, 20 bp, Artificial Sequence
  Synthetic Oligonucleotide
  modified_base (2)...(2): m5c
  modified_base (5)...(5): m5c
tcgtcgttcccccccccccc

SEQ ID NO: 326 - DNA, 20 bp, Artificial Sequence
  Synthetic Oligonucleotide
  modified_base (2)...(2): m5c
tcgtcgttcccccccccccc

SEQ ID NO: 327 - DNA, 20 bp, Artificial Sequence
  Synthetic Oligonucleotide
  modified_base (5)...(5): m5c
tcgtcgttcccccccccccc

SEQ ID NO: 328 - DNA, 20 bp, Artificial Sequence
  Synthetic Oligonucleotide
tcgtcgctcccccccccccc

SEQ ID NO: 329 - DNA, 20 bp, Artificial Sequence
  Synthetic Oligonucleotide
tcgtcggtcccccccccccc

SEQ ID NO: 330 - DNA, 20 bp, Artificial Sequence
  Synthetic Oligonucleotide
tcggcgttcccccccccccc

SEQ ID NO: 331 - DNA, 20 bp, Artificial Sequence
  Synthetic Oligonucleotide
ggccttttcccccccccccc

SEQ ID NO: 332 - DNA, 24 bp, Artificial Sequence
  Synthetic Oligonucleotide
tcgtcgttttgacgttttgtcgtt

SEQ ID NO: 333 - DNA, 24 bp, Artificial Sequence
  Synthetic Oligonucleotide
tcgtcgttttgacgttttgacgtt

SEQ ID NO: 334 - DNA, 20 bp, Artificial Sequence
  Synthetic Oligonucleotide
ccgtcgttcccccccccccc

SEQ ID NO: 335 - DNA, 20 bp, Artificial Sequence
  Synthetic Oligonucleotide
gcgtcgttcccccccccccc

SEQ ID NO: 336 - DNA, 20 bp, Artificial Sequence
  Synthetic Oligonucleotide
tcgtcattcccccccccccc

SEQ ID NO: 337 - DNA, 20 bp, Artificial Sequence
  Synthetic Oligonucleotide
acgtcgttcccccccccccc

SEQ ID NO: 338 - DNA, 20 bp, Artificial Sequence
  Synthetic Oligonucleotide
ctgtcgttcccccccccccc

SEQ ID NO: 339 - DNA, 24 bp, Artificial Sequence
  Synthetic Oligonucleotide
  misc_feature (1)...(1): biotinylated
tttttcgtcgttcccccccccccc

SEQ ID NO: 340 - DNA, 20 bp, Artificial Sequence
  Synthetic Oligonucleotide
  misc_feature (20)...(20): biotinylated
tcgtcgttcccccccccccc

SEQ ID NO: 341 - DNA, 24 bp, Artificial Sequence
  Synthetic Oligonucleotide
  misc_feature (24)...(24): biotinylated
tcgtcgttttgtcgttttgtcgtt

SEQ ID NO: 342 - DNA, 20 bp, Artificial Sequence
  Synthetic Oligonucleotide
tccagttccttcctcagtct

SEQ ID NO: 343 - DNA, 24 bp, Artificial Sequence
  Synthetic Oligonucleotide
  modified_base (2)...(2): m5c
tcgtcgttttgtcgttttgtcgtt

SEQ ID NO: 344 - DNA, 15 bp, Artificial Sequence
  Synthetic Oligonucleotide
tcctggaggggaagt

SEQ ID NO: 345 - DNA, 15 bp, Artificial Sequence
  Synthetic Oligonucleotide
tcctgaaaaggaagt

SEQ ID NO: 346 - DNA, 17 bp, Artificial Sequence
  Synthetic Oligonucleotide
tcgtcgttccccccccc

SEQ ID NO: 347 - DNA, 24 bp, Artificial Sequence
  Synthetic Oligonucleotide
  modified_base (2)...(2): m5c
  modified_base (5)...(5): m5c
  modified_base (13)...(13): m5c
  modified_base (21)...(21): m5c
tcgtcgttttgtcgttttgtcgtt

SEQ ID NO: 348 - DNA, 20 bp, Artificial Sequence
  Synthetic Oligonucleotide
ggggtcaagcttgagggggg

SEQ ID NO: 349 - DNA, 20 bp, Artificial Sequence
  Synthetic Oligonucleotide
tgctgcttcccccccccccc

SEQ ID NO: 350 - DNA, 14 bp, Artificial Sequence
  Synthetic Oligonucleotide
tcgtcgtcgtcgtt

SEQ ID NO: 351 - DNA, 14 bp, Artificial Sequence
  Synthetic Oligonucleotide
tcgtcgtcgtcgtt

SEQ ID NO: 352 - DNA, 14 bp, Artificial Sequence
  Synthetic Oligonucleotide
tcgtcgtcgtcgtt

SEQ ID NO: 353 - DNA, 10 bp, Artificial Sequence
  Synthetic Oligonucleotide
tcaacgttga

SEQ ID NO: 354 - DNA, 20 bp, Artificial Sequence
  Synthetic Oligonucleotide
atagttttccatttttttac

SEQ ID NO: 355 - DNA, 20 bp, Artificial Sequence
  Synthetic Oligonucleotide
aatagtcgccatcgcgcgac

SEQ ID NO: 356 - DNA, 20 bp, Artificial Sequence
  Synthetic Oligonucleotide
aatagtcgccatcccgggac

SEQ ID NO: 357 - DNA, 20 bp, Artificial Sequence
  Synthetic Oligonucleotide
aatagtcgccatcccccccc

SEQ ID NO: 358 - DNA, 24 bp, Artificial Sequence
  Synthetic Oligonucleotide
tgctgcttttgtgcttttgtgctt

SEQ ID NO: 359 - DNA, 24 bp, Artificial Sequence
  Synthetic Oligonucleotide
ctgtgctttctgtgtttttctgtg

SEQ ID NO: 360 - DNA, 24 bp, Artificial Sequence
  Synthetic Oligonucleotide
ctaatctttctaatttttttctaa

SEQ ID NO: 361 - DNA, 26 bp, Artificial Sequence
  Synthetic Oligonucleotide
tcgtcgttggtgtcgttggtgtcgtt

SEQ ID NO: 362 - DNA, 24 bp, Artificial Sequence
  Synthetic Oligonucleotide
tcgtcgttggttgtcgttttggtt

SEQ ID NO: 363 - DNA, 24 bp, Artificial Sequence
  Synthetic Oligonucleotide
accatggacgagctgtttcccctc

SEQ ID NO: 364 - DNA, 20 bp, Artificial Sequence
  Synthetic Oligonucleotide
tcgtcgttttgcgtgcgttt

SEQ ID NO: 365 - DNA, 20 bp, Artificial Sequence
  Synthetic Oligonucleotide
ctgtaagtgagcttggagag

SEQ ID NO: 366 - DNA, 18 bp, Artificial Sequence
  Synthetic Oligonucleotide
gagaacgctggaccttcc

SEQ ID NO: 367 - DNA, 20 bp, Artificial Sequence
  Synthetic Oligonucleotide
cgggcgactcagtctatcgg

SEQ ID NO: 368 - DNA, 37 bp, Artificial Sequence
  Synthetic Oligonucleotide
gttctcagataaagcggaaccagcaacagacacagaa

SEQ ID NO: 369 - DNA, 37 bp, Artificial Sequence
  Synthetic Oligonucleotide
ttctgtgtctgttgctggttccgctttatctgagaac

SEQ ID NO: 370 - DNA, 23 bp, Artificial Sequence
  Synthetic Oligonucleotide
cagacacagaagcccgatagacg

SEQ ID NO: 371 - DNA, 20 bp, Artificial Sequence
  Synthetic Oligonucleotide
agacagacacgaaacgaccg

SEQ ID NO: 372 - DNA, 20 bp, Artificial Sequence
  Synthetic Oligonucleotide
gtctgtcccatgatctcgaa

SEQ ID NO: 373 - DNA, 20 bp, Artificial Sequence
  Synthetic Oligonucleotide
gctggccagcttacctcccg

SEQ ID NO: 374 - DNA, 21 bp, Artificial Sequence
  Synthetic Oligonucleotide
ggggcctctatacaacctggg

SEQ ID NO: 375 - DNA, 18 bp, Artificial Sequence
  Synthetic Oligonucleotide
ggggtccctgagactgcc

SEQ ID NO: 376 - DNA, 20 bp, Artificial Sequence
  Synthetic Oligonucleotide
gagaacgctggaccttccat

SEQ ID NO: 377 - DNA, 20 bp, Artificial Sequence
  Synthetic Oligonucleotide
tccatgtcggtcctgatgct

SEQ ID NO: 378 - DNA, 20 bp, Artificial Sequence
  Synthetic Oligonucleotide
ctcttgcgacctggaaggta

SEQ ID NO: 379 - DNA, 20 bp, Artificial Sequence
  Synthetic Oligonucleotide
aggtacagccaggactacga

SEQ ID NO: 380 - DNA, 24 bp, Artificial Sequence
  Synthetic Oligonucleotide
accatggacgacctgtttcccctc

SEQ ID NO: 381 - DNA, 24 bp, Artificial Sequence
  Synthetic Oligonucleotide
accatggattacctttttcccctt

SEQ ID NO: 382 - DNA, 20 bp, Artificial Sequence
  Synthetic Oligonucleotide
atggaaggtccagcgttctc

SEQ ID NO: 383 - DNA, 20 bp, Artificial Sequence
  Synthetic Oligonucleotide
agcatcaggaccgacatgga

SEQ ID NO: 384 - DNA, 20 bp, Artificial Sequence
  Synthetic Oligonucleotide
ctctccaagctcacttacag

SEQ ID NO: 385 - DNA, 21 bp, Artificial Sequence
  Synthetic Oligonucleotide
tccctgagactgccccacctt

SEQ ID NO: 386 - DNA, 20 bp, Artificial Sequence
  Synthetic Oligonucleotide
gccaccaaaacttgtccatg

SEQ ID NO: 387 - DNA, 20 bp, Artificial Sequence
  Synthetic Oligonucleotide
gtccatggcgtgcgggatga

SEQ ID NO: 388 - DNA, 19 bp, Artificial Sequence
  Synthetic Oligonucleotide
cctctatacaacctgggac

SEQ ID NO: 389 - DNA, 20 bp, Artificial Sequence
  Synthetic Oligonucleotide
gcgctaccggtagcctgagt

SEQ ID NO: 390 - DNA, 35 bp, Artificial Sequence
  Synthetic Oligonucleotide
cgactgccgaacaggatatcggtgatcagcactgg

SEQ ID NO: 391 - DNA, 35 bp, Artificial Sequence
  Synthetic Oligonucleotide
ccagtgctgatcaccgatatcctgttcggcagtcg

SEQ ID NO: 392 - DNA, 17 bp, Artificial Sequence
  Synthetic Oligonucleotide
ccaggttgtatagaggc

SEQ ID NO: 393 - DNA, 18 bp, Artificial Sequence
  Synthetic Oligonucleotide
tctcccagcgtacgccat

SEQ ID NO: 394 - DNA, 18 bp, Artificial Sequence
  Synthetic Oligonucleotide
tctcccagcgtgcgtttt

SEQ ID NO: 395 - DNA, 18 bp, Artificial Sequence
  Synthetic Oligonucleotide
tctcccgacgtgcgccat

SEQ ID NO: 396 - DNA, 18 bp, Artificial Sequence
  Synthetic Oligonucleotide
tctcccgtcgtgcgccat

SEQ ID NO: 397 - DNA, 20 bp, Artificial Sequence
  Synthetic Oligonucleotide
ataatcgtcgttcaagcaag

SEQ ID NO: 398 - DNA, 23 bp, Artificial Sequence
  Synthetic Oligonucleotide
tcgtcgttttgtcgttttgtcgt

SEQ ID NO: 399 - DNA, 24 bp, Artificial Sequence
  Synthetic Oligonucleotide
tcgtcgttttgtcgttttgtcgtt

SEQ ID NO: 400 - DNA, 24 bp, Artificial Sequence
  Synthetic Oligonucleotide
tcntcgtnttntcgtnttntcgtn

SEQ ID NO: 401 - DNA, 17 bp, Artificial Sequence
  Synthetic Oligonucleotide
tctcccagcgtcgccat

SEQ ID NO: 402 - DNA, 17 bp, Artificial Sequence
  Synthetic Oligonucleotide
tctcccatcgtcgccat

SEQ ID NO: 403 - DNA, 21 bp, Artificial Sequence
  Synthetic Oligonucleotide
ataatcgtgcgttcaagaaag

SEQ ID NO: 404 - DNA, 20 bp, Artificial Sequence
  Synthetic Oligonucleotide
ataatcgacgttcccccccc

SEQ ID NO: 405 - DNA, 20 bp, Artificial Sequence
  Synthetic Oligonucleotide
tctatcgacgttcaagcaag

SEQ ID NO: 406 - DNA, 14 bp, Artificial Sequence
  Synthetic Oligonucleotide
tcctgacggggagt

SEQ ID NO: 407 - DNA, 19 bp, Artificial Sequence
  Synthetic Oligonucleotide
tccatgacgttcctgatcc

SEQ ID NO: 408 - DNA, 15 bp, Artificial Sequence
  Synthetic Oligonucleotide
tcctggcgtggaagt

SEQ ID NO: 409 - DNA, 24 bp, Artificial Sequence
  Synthetic Oligonucleotide
agcagctttagagctttagagctt

SEQ ID NO: 410 - DNA, 24 bp, Artificial Sequence
  Synthetic Oligonucleotide
cccccccccccccccccccccccc

SEQ ID NO: 411 - DNA, 32 bp, Artificial Sequence
  Synthetic Oligonucleotide
tcgtcgttttgtcgttttgtcgttttgtcgtt

SEQ ID NO: 412 - DNA, 28 bp, Artificial Sequence
  Synthetic Oligonucleotide
tcgtcgttttttgtcgttttttgtcgtt

SEQ ID NO: 413 - DNA, 20 bp, Artificial Sequence
  Synthetic Oligonucleotide
tcgtcgtttttttttttttt

SEQ ID NO: 414 - DNA, 20 bp, Artificial Sequence
  Synthetic Oligonucleotide
tttttcaacgttgatttttt

SEQ ID NO: 415 - DNA, 24 bp, Artificial Sequence
  Synthetic Oligonucleotide
tttttttttttttttttttttttt

SEQ ID NO: 416 - DNA, 20 bp, Artificial Sequence
  Synthetic Oligonucleotide
ggggtcgtcgttttgggggg

SEQ ID NO: 417 - DNA, 24 bp, Artificial Sequence
  Synthetic Oligonucleotide
tcgtcgttttgtcgttttgggggg

SEQ ID NO: 418 - DNA, 27 bp, Artificial Sequence
  Synthetic Oligonucleotide
tcgtcgctgtctccgcttcttcttgcc

SEQ ID NO: 419 - DNA, 15 bp, Artificial Sequence
  Synthetic Oligonucleotide
tcgtcgctgtctccg

SEQ ID NO: 420 - DNA, 17 bp, Artificial Sequence
  Synthetic Oligonucleotide
ccaggttgtatagaggc

SEQ ID NO: 421 - DNA, 17 bp, Artificial Sequence
  Synthetic Oligonucleotide
gctagacgttagcgtga

SEQ ID NO: 422 - DNA, 20 bp, Artificial Sequence
  Synthetic Oligonucleotide
ggagctcttcgaacgccata

SEQ ID NO: 423 - DNA, 20 bp, Artificial Sequence
  Synthetic Oligonucleotide
tctccatgatggttttatcg

SEQ ID NO: 424 - DNA, 21 bp, Artificial Sequence
  Synthetic Oligonucleotide
aaggtggggcagtctcaggga

SEQ ID NO: 425 - DNA, 20 bp, Artificial Sequence
  Synthetic Oligonucleotide
atcggaggactggcgcgccg

SEQ ID NO: 426 - DNA, 20 bp, Artificial Sequence
  Synthetic Oligonucleotide
ttaggacaaggtctagggtg

SEQ ID NO: 427 - DNA, 20 bp, Artificial Sequence
  Synthetic Oligonucleotide
accacaacgagaggaacgca

SEQ ID NO: 428 - DNA, 20 bp, Artificial Sequence
  Synthetic Oligonucleotide
ggcagtgcaggctcaccggg

SEQ ID NO: 429 - DNA, 17 bp, Artificial Sequence
  Synthetic Oligonucleotide
gaaccttccatgctgtt

SEQ ID NO: 430 - DNA, 20 bp, Artificial Sequence
  Synthetic Oligonucleotide
gcttggagggcctgtaagtg

SEQ ID NO: 431 - DNA, 12 bp, Artificial Sequence
  Synthetic Oligonucleotide
gtagccttccta

SEQ ID NO: 432 - DNA, 14 bp, Artificial Sequence
  Synthetic Oligonucleotide
cggtagccttccta

SEQ ID NO: 433 - DNA, 16 bp, Artificial Sequence
  Synthetic Oligonucleotide
cacggtagccttccta

SEQ ID NO: 434 - DNA, 18 bp, Artificial Sequence
  Synthetic Oligonucleotide
agcacggtagccttccta

SEQ ID NO: 435 - DNA, 18 bp, Artificial Sequence
  Synthetic Oligonucleotide
gaacgctggaccttccat

SEQ ID NO: 436 - DNA, 10 bp, Artificial Sequence
  Synthetic Oligonucleotide
gaccttccat

SEQ ID NO: 437 - DNA, 12 bp, Artificial Sequence
  Synthetic Oligonucleotide
tggaccttccat

SEQ ID NO: 438 - DNA, 14 bp, Artificial Sequence
  Synthetic Oligonucleotide
gctggaccttccat

SEQ ID NO: 439 - DNA, 16 bp, Artificial Sequence
  Synthetic Oligonucleotide
acgctggaccttccat

SEQ ID NO: 440 - DNA, 20 bp, Artificial Sequence
  Synthetic Oligonucleotide
taagctctgtcaacgccagg

SEQ ID NO: 441 - DNA, 22 bp, Artificial Sequence
  Synthetic Oligonucleotide
gagaacgctggaccttccatgt

SEQ ID NO: 442 - DNA, 21 bp, Artificial Sequence
  Synthetic Oligonucleotide
ttcatgccttgcaaaatggcg

SEQ ID NO: 443 - DNA, 20 bp, Artificial Sequence
  Synthetic Oligonucleotide
tgctagctgtgcctgtacct

SEQ ID NO: 444 - DNA, 20 bp, Artificial Sequence
  Synthetic Oligonucleotide
agcatcaggaccgacatgga

SEQ ID NO: 445 - DNA, 22 bp, Artificial Sequence
  Synthetic Oligonucleotide
gaccttccatgtcggtcctgat

SEQ ID NO: 446 - DNA, 20 bp, Artificial Sequence
  Synthetic Oligonucleotide
acaaccacgagaacgggaac

SEQ ID NO: 447 - DNA, 20 bp, Artificial Sequence
  Synthetic Oligonucleotide
gaaccttccatgctgttccg

SEQ ID NO: 448 - DNA, 20 bp, Artificial Sequence
  Synthetic Oligonucleotide
caatcaatctgaggagaccc

SEQ ID NO: 449 - DNA, 20 bp, Artificial Sequence
  Synthetic Oligonucleotide
tcagctctggtactttttca

SEQ ID NO: 450 - DNA, 20 bp, Artificial Sequence
  Synthetic Oligonucleotide
tggttacggtctgtcccatg

SEQ ID NO: 451 - DNA, 20 bp, Artificial Sequence
  Synthetic Oligonucleotide
gtctatcggaggactggcgc

SEQ ID NO: 452 - DNA, 20 bp, Artificial Sequence
  Synthetic Oligonucleotide
cattttacgggcgggcgggc

SEQ ID NO: 453 - DNA, 20 bp, Artificial Sequence
  Synthetic Oligonucleotide
gaggggaccattttacgggc

SEQ ID NO: 454 - DNA, 20 bp, Artificial Sequence
  Synthetic Oligonucleotide
tgtccagccgaggggaccat

SEQ ID NO: 455 - DNA, 20 bp, Artificial Sequence
  Synthetic Oligonucleotide
cgggcttacggcggatgctg

SEQ ID NO: 456 - DNA, 20 bp, Artificial Sequence
  Synthetic Oligonucleotide
tggaccttctatgtcggtcc

SEQ ID NO: 457 - DNA, 20 bp, Artificial Sequence
  Synthetic Oligonucleotide
tgtcccatgtttttagaagc

SEQ ID NO: 458 - DNA, 20 bp, Artificial Sequence
  Synthetic Oligonucleotide
gtggttacggtcgtgcccat

SEQ ID NO: 459 - DNA, 20 bp, Artificial Sequence
  Synthetic Oligonucleotide
cctccaaatgaaagaccccc

SEQ ID NO: 460 - DNA, 20 bp, Artificial Sequence
  Synthetic Oligonucleotide
ttgtactctccatgatggtt

SEQ ID NO: 461 - DNA, 20 bp, Artificial Sequence
  Synthetic Oligonucleotide
ttccatgctgttccggctgg

SEQ ID NO: 462 - DNA, 20 bp, Artificial Sequence
  Synthetic Oligonucleotide
gaccttctatgtcggtcctg

SEQ ID NO: 463 - DNA, 20 bp, Artificial Sequence
  Synthetic Oligonucleotide
gagaccgctcgaccttcgat

SEQ ID NO: 464 - DNA, 20 bp, Artificial Sequence
  Synthetic Oligonucleotide
ttgccccatattttagaaac

SEQ ID NO: 465 - DNA, 18 bp, Artificial Sequence
  Synthetic Oligonucleotide
ttgaaactgaggtgggac

SEQ ID NO: 466 - DNA, 21 bp, Artificial Sequence
  Synthetic Oligonucleotide
ctatcggaggactggcgcgcc

SEQ ID NO: 467 - DNA, 20 bp, Artificial Sequence
  Synthetic Oligonucleotide
cttggagggcctcccggcgg

SEQ ID NO: 468 - DNA, 20 bp, Artificial Sequence
  Synthetic Oligonucleotide
gctgaaccttccatgctgtt

SEQ ID NO: 469 - DNA, 32 bp, Artificial Sequence
  Synthetic Oligonucleotide
tagaaacagcattcttcttttagggcagcaca

SEQ ID NO: 470 - DNA, 24 bp, Artificial Sequence
  Synthetic Oligonucleotide
agatggttctcagataaagcggaa

SEQ ID NO: 471 - DNA, 24 bp, Artificial Sequence
  Synthetic Oligonucleotide
ttccgctttatctgagaaccatct

SEQ ID NO: 472 - DNA, 23 bp, Artificial Sequence
  Synthetic Oligonucleotide
gtcccaggttgtatagaggctgc

SEQ ID NO: 473 - DNA, 20 bp, Artificial Sequence
  Synthetic Oligonucleotide
gcgccagtcctccgatagac

SEQ ID NO: 474 - DNA, 20 bp, Artificial Sequence
  Synthetic Oligonucleotide
ggtctgtcccatatttttag

SEQ ID NO: 475 - DNA, 20 bp, Artificial Sequence
  Synthetic Oligonucleotide
tttttcaacgttgagggggg

SEQ ID NO: 476 - DNA, 21 bp, Artificial Sequence
  Synthetic Oligonucleotide
tttttcaagcgttgatttttt

SEQ ID NO: 477 - DNA, 20 bp, Artificial Sequence
  Synthetic Oligonucleotide
ggggtcaacgttgatttttt

SEQ ID NO: 478 - DNA, 25 bp, Artificial Sequence
  Synthetic Oligonucleotide
ggggttttcaacgttttgagggggg

SEQ ID NO: 479 - DNA, 20 bp, Artificial Sequence
  Synthetic Oligonucleotide
ggttacggtctgtcccatat

SEQ ID NO: 480 - DNA, 20 bp, Artificial Sequence
  Synthetic Oligonucleotide
ctgtcccatatttttagaca

SEQ ID NO: 481 - DNA, 20 bp, Artificial Sequence
  Synthetic Oligonucleotide
accatcctgaggccattcgg

SEQ ID NO: 482 - DNA, 23 bp, Artificial Sequence
  Synthetic Oligonucleotide
cgtctatcgggcttctgtgtctg

SEQ ID NO: 483 - DNA, 21 bp, Artificial Sequence
  Synthetic Oligonucleotide
ggccatcccacattgaaagtt

SEQ ID NO: 484 - DNA, 22 bp, Artificial Sequence
  Synthetic Oligonucleotide
ccaaatatcggtggtcaagcac

SEQ ID NO: 485 - DNA, 22 bp, Artificial Sequence
  Synthetic Oligonucleotide
gtgcttgaccaccgatatttgg

SEQ ID NO: 486 - DNA, 26 bp, Artificial Sequence
  Synthetic Oligonucleotide
gtgctgatcaccgatatcctgttcgg

SEQ ID NO: 487 - DNA, 27 bp, Artificial Sequence
  Synthetic Oligonucleotide
ggccaactttcaatgtgggatggcctc

SEQ ID NO: 488 - DNA, 27 bp, Artificial Sequence
  Synthetic Oligonucleotide
ttccgccgaatggcctcaggatggtac

SEQ ID NO: 489 - DNA, 36 bp, Artificial Sequence
  Synthetic Oligonucleotide
tatagtccctgagactgccccaccttctcaacaacc

SEQ ID NO: 490 - DNA, 27 bp, Artificial Sequence
  Synthetic Oligonucleotide
gcagcctctatacaacctgggacggga

SEQ ID NO: 491 - DNA, 22 bp, Artificial Sequence
  Synthetic Oligonucleotide
ctatcggaggactggcgcgccg

SEQ ID NO: 492 - DNA, 21 bp, Artificial Sequence
  Synthetic Oligonucleotide
tatcggaggactggcgcgccg

SEQ ID NO: 493 - DNA, 21 bp, Artificial Sequence
  Synthetic Oligonucleotide
gatcggaggactggcgcgccg

SEQ ID NO: 494 - DNA, 26 bp, Artificial Sequence
  Synthetic Oligonucleotide
ccgaacaggatatcggtgatcagcac

SEQ ID NO: 495 - DNA, 24 bp, Artificial Sequence
  Synthetic Oligonucleotide
ttttggggtcaacgttgagggggg

SEQ ID NO: 496 - DNA, 20 bp, Artificial Sequence
  Synthetic Oligonucleotide
ggggtcaacgttgagggggg

SEQ ID NO: 497 - DNA, 20 bp, Artificial Sequence
  Synthetic Oligonucleotide
cgcgcgcgcgcgcgcgcgcg

SEQ ID NO: 498 - DNA, 20 bp, Artificial Sequence
  Synthetic Oligonucleotide
ggggcatgacgttcgggggg

SEQ ID NO: 499 - DNA, 20 bp, Artificial Sequence
  Synthetic Oligonucleotide
ggggcatgacgttcaaaaaa

SEQ ID NO: 500 - DNA, 20 bp, Artificial Sequence
  Synthetic Oligonucleotide
ggggcatgagcttcgggggg

SEQ ID NO: 501 - DNA, 20 bp, Artificial Sequence
  Synthetic Oligonucleotide
ggggcatgacgttcgggggg

SEQ ID NO: 502 - DNA, 20 bp, Artificial Sequence
  Synthetic Oligonucleotide
aaaacatgacgttcaaaaaa

SEQ ID NO: 503 - DNA, 20 bp, Artificial Sequence
  Synthetic Oligonucleotide
aaaacatgacgttcgggggg

SEQ ID NO: 504 - DNA, 20 bp, Artificial Sequence
  Synthetic Oligonucleotide
ggggcatgacgttcaaaaaa

SEQ ID NO: 505 - DNA, 24 bp, Artificial Sequence
  Synthetic Oligonucleotide
accatggacgatctgtttcccctc

SEQ ID NO: 506 - DNA, 24 bp, Artificial Sequence
  Synthetic Oligonucleotide
gccatggacgaactgttccccctc

SEQ ID NO: 507 - DNA, 20 bp, Artificial Sequence
  Synthetic Oligonucleotide
cccccccccccccccccccc

SEQ ID NO: 508 - DNA, 20 bp, Artificial Sequence
  Synthetic Oligonucleotide
gggggggggggggggggggg

SEQ ID NO: 509 - DNA, 20 bp, Artificial Sequence
  Synthetic Oligonucleotide
gctgtaaaatgaatcggccg

SEQ ID NO: 510 - DNA, 20 bp, Artificial Sequence
  Synthetic Oligonucleotide
ttcgggcggactcctccatt

SEQ ID NO: 511 - DNA, 20 bp, Artificial Sequence
  Synthetic Oligonucleotide
tatgccgcgcccggacttat

SEQ ID NO: 512 - DNA, 20 bp, Artificial Sequence
  Synthetic Oligonucleotide
ggggtaatcgatcagggggg

SEQ ID NO: 513 - DNA, 20 bp, Artificial Sequence
  Synthetic Oligonucleotide
tttgagaacgctggaccttc

SEQ ID NO: 514 - DNA, 20 bp, Artificial Sequence
  Synthetic Oligonucleotide
gatcgctgatctaatgctcg

SEQ ID NO: 515 - DNA, 20 bp, Artificial Sequence
  Synthetic Oligonucleotide
gtcggtcctgatgctgttcc

SEQ ID NO: 516 - DNA, 20 bp, Artificial Sequence
  Synthetic Oligonucleotide
tcgtcgtcagttcgctgtcg

SEQ ID NO: 517 - DNA, 18 bp, Artificial Sequence
  Synthetic Oligonucleotide
ctggaccttccatgtcgg

SEQ ID NO: 518 - DNA, 17 bp, Artificial Sequence
  Synthetic Oligonucleotide
gctcgttcagcgcgtct

SEQ ID NO: 519 - DNA, 16 bp, Artificial Sequence
  Synthetic Oligonucleotide
ctggaccttccatgtc

SEQ ID NO: 520 - DNA, 16 bp, Artificial Sequence
  Synthetic Oligonucleotide
cactgtccttcgtcga

SEQ ID NO: 521 - DNA, 20 bp, Artificial Sequence
  Synthetic Oligonucleotide
cgctggaccttccatgtcgg

SEQ ID NO: 522 - DNA, 20 bp, Artificial Sequence
  Synthetic Oligonucleotide
gctgagctcatgccgtctgc

SEQ ID NO: 523 - DNA, 20 bp, Artificial Sequence
  Synthetic Oligonucleotide
aacgctggaccttccatgtc

SEQ ID NO: 524 - DNA, 20 bp, Artificial Sequence
  Synthetic Oligonucleotide
tgcatgccgtacacagctct

SEQ ID NO: 525 - DNA, 20 bp, Artificial Sequence
  Synthetic Oligonucleotide
ccttccatgtcggtcctgat

SEQ ID NO: 526 - DNA, 20 bp, Artificial Sequence
  Synthetic Oligonucleotide
tactcttcggatcccttgcg

SEQ ID NO: 527 - DNA, 18 bp, Artificial Sequence
  Synthetic Oligonucleotide
ttccatgtcggtcctgat

SEQ ID NO: 528 - DNA, 18 bp, Artificial Sequence
  Synthetic Oligonucleotide
ctgattgctctctcgtga

SEQ ID NO: 529 - DNA, 20 bp, Artificial Sequence
  Synthetic Oligonucleotide
ggcgttattcctgactcgcc

SEQ ID NO: 530 - DNA, 22 bp, Artificial Sequence
  Synthetic Oligonucleotide
cctacgttgtatgcgcccagct

SEQ ID NO: 531 - DNA, 20 bp, Artificial Sequence
  Synthetic Oligonucleotide
ggggtaatcgatgagggggg

SEQ ID NO: 532 - DNA, 20 bp, Artificial Sequence
  Synthetic Oligonucleotide
ttcgggcggactcctccatt

SEQ ID NO: 533 - DNA, 20 bp, Artificial Sequence
  Synthetic Oligonucleotide
tttttttttttttttttttt

SEQ ID NO: 534 - DNA, 20 bp, Artificial Sequence
  Synthetic Oligonucleotide
gggggttttttttttggggg

SEQ ID NO: 535 - DNA, 20 bp, Artificial Sequence
  Synthetic Oligonucleotide
tttttggggggggggttttt

SEQ ID NO: 536 - DNA, 19 bp, Artificial Sequence
  Synthetic Oligonucleotide
ggggggggggggggggggt

SEQ ID NO: 537 - DNA, 20 bp, Artificial Sequence
  Synthetic Oligonucleotide
aaaaaaaaaaaaaaaaaaaa

SEQ ID NO: 538 - DNA, 20 bp, Artificial Sequence
  Synthetic Oligonucleotide
cccccaaaaaaaaaaccccc

SEQ ID NO: 539 - DNA, 20 bp, Artificial Sequence
  Synthetic Oligonucleotide
aaaaaccccccccccaaaaa

SEQ ID NO: 540 - DNA, 27 bp, Artificial Sequence
  Synthetic Oligonucleotide
tttgaattcaggactggtgaggttgag

SEQ ID NO: 541 - DNA, 27 bp, Artificial Sequence
  Synthetic Oligonucleotide
tttgaatcctcagcggtctccagtggc

SEQ ID NO: 542 - DNA, 45 bp, Artificial Sequence
  Synthetic Oligonucleotide
aattctctatcggggcttctgtgtctgttgctggttccgctttat

SEQ ID NO: 543 - DNA, 45 bp, Artificial Sequence
  Synthetic Oligonucleotide
ctagataaagcggaaccagcaacagacacagaagccccgatagag

SEQ ID NO: 544 - DNA, 28 bp, Artificial Sequence
  Synthetic Oligonucleotide
ttttctagagaggtgcacaatgctctgg

SEQ ID NO: 545 - DNA, 29 bp, Artificial Sequence
  Synthetic Oligonucleotide
tttgaattccgtgtacagaagcgagaagc

SEQ ID NO: 546 - DNA, 31 bp, Artificial Sequence
  Synthetic Oligonucleotide
tttgcggccgctagacttaacctgagagata

SEQ ID NO: 547 - DNA, 29 bp, Artificial Sequence
  Synthetic Oligonucleotide
tttgggcccacgagagacagagacacttc

SEQ ID NO: 548 - DNA, 29 bp, Artificial Sequence
  Synthetic Oligonucleotide
tttgggcccgcttctcgcttctgtacacg

SEQ ID NO: 549 - DNA, 20 bp, Artificial Sequence
  Synthetic Oligonucleotide
gagaacgctggaccttccat

SEQ ID NO: 550 - DNA, 20 bp, Artificial Sequence
  Synthetic Oligonucleotide
tccatgtcggtcctgatgct

SEQ ID NO: 551 - DNA, 6 bp, Artificial Sequence
  Synthetic Oligonucleotide
ctgtcg

SEQ ID NO: 552 - DNA, 6 bp, Artificial Sequence
  Synthetic Oligonucleotide
tcgtga

SEQ ID NO: 553 - DNA, 6 bp, Artificial Sequence
  Synthetic Oligonucleotide
cgtcga

SEQ ID NO: 554 - DNA, 6 bp, Artificial Sequence
  Synthetic Oligonucleotide
agtgct

SEQ ID NO: 555 - DNA, 6 bp, Artificial Sequence
  Synthetic Oligonucleotide
ctgtcg

SEQ ID NO: 556 - DNA, 6 bp, Artificial Sequence
  Synthetic Oligonucleotide
agtgct

SEQ ID NO: 557 - DNA, 6 bp, Artificial Sequence
  Synthetic Oligonucleotide
cgtcga

SEQ ID NO: 558 - DNA, 6 bp, Artificial Sequence
  Synthetic Oligonucleotide
tcgtga

SEQ ID NO: 559 - DNA, 20 bp, Artificial Sequence
  Synthetic Oligonucleotide
gagaacgctccagcttcgat

SEQ ID NO: 560 - DNA, 17 bp, Artificial Sequence
  Synthetic Oligonucleotide
gctagacgtaagcgtga

SEQ ID NO: 561 - DNA, 20 bp, Artificial Sequence
  Synthetic Oligonucleotide
gagaacgctcgaccttccat

SEQ ID NO: 562 - DNA, 21 bp, Artificial Sequence
  Synthetic Oligonucleotide
gagaacgctggacctatccat

SEQ ID NO: 563 - DNA, 17 bp, Artificial Sequence
  Synthetic Oligonucleotide
gctagaggttagcgtga

SEQ ID NO: 564 - DNA, 19 bp, Artificial Sequence
  Synthetic Oligonucleotide
gagaacgctggacttccat

SEQ ID NO: 565 - DNA, 17 bp, Artificial Sequence
  Synthetic Oligonucleotide
tcacgctaacgtctagc

SEQ ID NO: 566 - DNA, 17 bp, Artificial Sequence
  Synthetic Oligonucleotide
  misc_feature (1)...(1): biotinylated
gctagacgttagcgtga

SEQ ID NO: 567 - DNA, 20 bp, Artificial Sequence
  Synthetic Oligonucleotide
atggaaggtcgagcgttctc

SEQ ID NO: 568 - DNA, 20 bp, Artificial Sequence
  Synthetic Oligonucleotide
gagaacgctggaccttcgat

SEQ ID NO: 569 - DNA, 20 bp, Artificial Sequence
  Synthetic Oligonucleotide
gagaacgatggaccttccat

SEQ ID NO: 570 - DNA, 17 bp, Artificial Sequence
  Synthetic Oligonucleotide
gagaacgctggatccat

SEQ ID NO: 571 - DNA, 20 bp, Artificial Sequence
  Synthetic Oligonucleotide
gagaacgctccagcactgat

SEQ ID NO: 572 - DNA, 20 bp, Artificial Sequence
  Synthetic Oligonucleotide
tccatgtcggtcctgctgat

SEQ ID NO: 573 - DNA, 20 bp, Artificial Sequence
  Synthetic Oligonucleotide
atgtcctcggtcctgatgct

SEQ ID NO: 574 - DNA, 20 bp, Artificial Sequence
  Synthetic Oligonucleotide
gagaacgctccaccttccat

SEQ ID NO: 575 - DNA, 20 bp, Artificial Sequence
  Synthetic Oligonucleotide
gagaacgctggaccttcgta

SEQ ID NO: 576 - DNA, 20 bp, Artificial Sequence
  Synthetic Oligonucleotide
  misc_feature (1)...(1): biotinylated
atggaaggtccagcgttctc

SEQ ID NO: 577 - DNA, 6 bp, Artificial Sequence
  Synthetic Oligonucleotide
tcctga

SEQ ID NO: 578 - DNA, 8 bp, Artificial Sequence
  Synthetic Oligonucleotide
tcaacgtt

SEQ ID NO: 579 - DNA, 6 bp, Artificial Sequence
  Synthetic Oligonucleotide
aacgtt

SEQ ID NO: 580 - DNA, 8 bp, Artificial Sequence
  Synthetic Oligonucleotide
aacgttga

SEQ ID NO: 581 - DNA, 17 bp, Artificial Sequence
  Synthetic Oligonucleotide
tcacgctaacctctagc

SEQ ID NO: 582 - DNA, 20 bp, Artificial Sequence
  Synthetic Oligonucleotide
gagaacgctggaccttgcat

SEQ ID NO: 583 - DNA, 14 bp, Artificial Sequence
  Synthetic Oligonucleotide
gctggaccttccat

SEQ ID NO: 584 - DNA, 22 bp, Artificial Sequence
  Synthetic Oligonucleotide
gagaacgctggacctcatccat

SEQ ID NO: 585 - DNA, 23 bp, Artificial Sequence
  Synthetic Oligonucleotide
gagaacgctggacgctcatccat

SEQ ID NO: 586 - DNA, 15 bp, Artificial Sequence
  Synthetic Oligonucleotide
aacgttgaggggcat

SEQ ID NO: 587 - DNA, 15 bp, Artificial Sequence
  Synthetic Oligonucleotide
atgcccctcaacgtt

SEQ ID NO: 588 - DNA, 10 bp, Artificial Sequence
  Synthetic Oligonucleotide
tcaacgttga

SEQ ID NO: 589 - DNA, 7 bp, Artificial Sequence
  Synthetic Oligonucleotide
caacgtt

SEQ ID NO: 590 - DNA, 10 bp, Artificial Sequence
  Synthetic Oligonucleotide
acaacgttga

SEQ ID NO: 591 - DNA, 6 bp, Artificial Sequence
  Synthetic Oligonucleotide
tcacgt

SEQ ID NO: 592 - DNA, 8 bp, Artificial Sequence
  Synthetic Oligonucleotide
tcaagctt

SEQ ID NO: 593 - DNA, 6 bp, Artificial Sequence
  Synthetic Oligonucleotide
tcgtca

SEQ ID NO: 594 - DNA, 8 bp, Artificial Sequence
  Synthetic Oligonucleotide
aggatatc

SEQ ID NO: 595 - DNA, 8 bp, Artificial Sequence
  Synthetic Oligonucleotide
tagacgtc

SEQ ID NO: 596 - DNA, 8 bp, Artificial Sequence
  Synthetic Oligonucleotide
gacgtcat

SEQ ID NO: 597 - DNA, 8 bp, Artificial Sequence
  Synthetic Oligonucleotide
ccatcgat

SEQ ID NO: 598 - DNA, 8 bp, Artificial Sequence
  Synthetic Oligonucleotide
atcgatgt

SEQ ID NO: 599 - DNA, 8 bp, Artificial Sequence
  Synthetic Oligonucleotide
atgcatgt

SEQ ID NO: 600 - DNA, 8 bp, Artificial Sequence
  Synthetic Oligonucleotide
ccatgcat

SEQ ID NO: 601 - DNA, 8 bp, Artificial Sequence
  Synthetic Oligonucleotide
agcgctga

SEQ ID NO: 602 - DNA, 8 bp, Artificial Sequence
  Synthetic Oligonucleotide
tcagcgct

SEQ ID NO: 603 - DNA, 8 bp, Artificial Sequence
  Synthetic Oligonucleotide
ccttcgat

SEQ ID NO: 604 - DNA, 18 bp, Artificial Sequence
  Synthetic Oligonucleotide
gtgccggggtctccgggc

SEQ ID NO: 605 - DNA, 18 bp, Artificial Sequence
  Synthetic Oligonucleotide
gctgtggggcggctcctg

SEQ ID NO: 606 - DNA, 8 bp, Artificial Sequence
  Synthetic Oligonucleotide
  misc_feature (1)...(1): biotinylated
tcaacgtt

SEQ ID NO: 607 - DNA, 8 bp, Artificial Sequence
  Synthetic Oligonucleotide
  misc_feature (1)...(1): fluorescein isothiocyanate conjugate
tcaacgtt

SEQ ID NO: 608 - DNA, 8 bp, Artificial Sequence
  Synthetic Oligonucleotide
  misc_feature (1)...(1): fluorescein isothiocyanate conjugate
aacgttga

SEQ ID NO: 609 - DNA, 7 bp, Artificial Sequence
  Synthetic Oligonucleotide
tcaacgt

SEQ ID NO: 610 - DNA, 7 bp, Artificial Sequence
  Synthetic Oligonucleotide
aacgttg

SEQ ID NO: 611 - DNA, 6 bp, Artificial Sequence
  Synthetic Oligonucleotide
cgacga

SEQ ID NO: 612 - DNA, 5 bp, Artificial Sequence
  Synthetic Oligonucleotide
tcgga

SEQ ID NO: 613 - DNA, 8 bp, Artificial Sequence
  Synthetic Oligonucleotide
agaacgtt

SEQ ID NO: 614 - DNA, 8 bp, Artificial Sequence
  Synthetic Oligonucleotide
tcatcgat

SEQ ID NO: 615 - DNA, 8 bp, Artificial Sequence
  Synthetic Oligonucleotide
taaacgtt

SEQ ID NO: 616 - DNA, 8 bp, Artificial Sequence
  Synthetic Oligonucleotide
ccaacgtt

SEQ ID NO: 617 - DNA, 6 bp, Artificial Sequence
  Synthetic Oligonucleotide
gctcga

SEQ ID NO: 618 - DNA, 6 bp, Artificial Sequence
  Synthetic Oligonucleotide
cgacgt

SEQ ID NO: 619 - DNA, 6 bp, Artificial Sequence
  Synthetic Oligonucleotide
acgtgt

SEQ ID NO: 620 - DNA, 6 bp, Artificial Sequence
  Synthetic Oligonucleotide
cgttcg

SEQ ID NO: 621 - DNA, 20 bp, Artificial Sequence
  Synthetic Oligonucleotide
gagcaagctggaccttccat

SEQ ID NO: 622 - DNA, 6 bp, Artificial Sequence
  Synthetic Oligonucleotide
cgcgta

SEQ ID NO: 623 - DNA, 6 bp, Artificial Sequence
  Synthetic Oligonucleotide
cgtacg

SEQ ID NO: 624 - DNA, 8 bp, Artificial Sequence
  Synthetic Oligonucleotide
tcaccggt

SEQ ID NO: 625 - DNA, 20 bp, Artificial Sequence
  Synthetic Oligonucleotide
caagagatgctaacaatgca

SEQ ID NO: 626 - DNA, 20 bp, Artificial Sequence
  Synthetic Oligonucleotide
acccatcaatagctctgtgc

SEQ ID NO: 627 - DNA, 8 bp, Artificial Sequence
  Synthetic Oligonucleotide
tcgacgtc

SEQ ID NO: 628 - DNA, 8 bp, Artificial Sequence
  Synthetic Oligonucleotide
ctagcgct

SEQ ID NO: 629 - DNA, 8 bp, Artificial Sequence
  Synthetic Oligonucleotide
taagcgct

SEQ ID NO: 630 - DNA, 13 bp, Artificial Sequence
  Synthetic Oligonucleotide
tcgcgaattcgcg

SEQ ID NO: 631 - DNA, 19 bp, Artificial Sequence
  Synthetic Oligonucleotide
atggaaggtccagcgttct

SEQ ID NO: 632 - DNA, 17 bp, Artificial Sequence
  Synthetic Oligonucleotide
actggacgttagcgtga

SEQ ID NO: 633 - DNA, 18 bp, Artificial Sequence
  Synthetic Oligonucleotide
cgcctggggctggtctgg

SEQ ID NO: 634 - DNA, 18 bp, Artificial Sequence
  Synthetic Oligonucleotide
gtgtcggggtctccgggc

SEQ ID NO: 635 - DNA, 18 bp, Artificial Sequence
  Synthetic Oligonucleotide
gtgccggggtctccgggc

SEQ ID NO: 636 - DNA, 18 bp, Artificial Sequence
  Synthetic Oligonucleotide
cgccgtcgcggcggttgg

SEQ ID NO: 637 - DNA, 21 bp, Artificial Sequence
  Synthetic Oligonucleotide
gaagttcacgttgaggggcat

SEQ ID NO: 638 - DNA, 21 bp, Artificial Sequence
  Synthetic Oligonucleotide
atctggtgagggcaagctatg

SEQ ID NO: 639 - DNA, 21 bp, Artificial Sequence
  Synthetic Oligonucleotide
gttgaaacccgagaacatcat

SEQ ID NO: 640 - DNA, 8 bp, Artificial Sequence
  Synthetic Oligonucleotide
gcaacgtt

SEQ ID NO: 641 - DNA, 8 bp, Artificial Sequence
  Synthetic Oligonucleotide
gtaacgtt

SEQ ID NO: 642 - DNA, 8 bp, Artificial Sequence
  Synthetic Oligonucleotide
cgaacgtt

SEQ ID NO: 643 - DNA, 8 bp, Artificial Sequence
  Synthetic Oligonucleotide
gaaacgtt

SEQ ID NO: 644 - DNA, 8 bp, Artificial Sequence
  Synthetic Oligonucleotide
caaacgtt

SEQ ID NO: 645 - DNA, 8 bp, Artificial Sequence
  Synthetic Oligonucleotide
ctaacgtt

SEQ ID NO: 646 - DNA, 8 bp, Artificial Sequence
  Synthetic Oligonucleotide
ggaacgtt

SEQ ID NO: 647 - DNA, 8 bp, Artificial Sequence
  Synthetic Oligonucleotide
tgaacgtt

SEQ ID NO: 648 - DNA, 8 bp, Artificial Sequence
  Synthetic Oligonucleotide
acaacgtt

SEQ ID NO: 649 - DNA, 8 bp, Artificial Sequence
  Synthetic Oligonucleotide
ttaacgtt

SEQ ID NO: 650 - DNA, 8 bp, Artificial Sequence
  Synthetic Oligonucleotide
aaaacgtt

SEQ ID NO: 651 - DNA, 8 bp, Artificial Sequence
  Synthetic Oligonucleotide
ataacgtt

SEQ ID NO: 652 - DNA, 8 bp, Artificial Sequence
  Synthetic Oligonucleotide
aacgttct

SEQ ID NO: 653 - DNA, 8 bp, Artificial Sequence
  Synthetic Oligonucleotide
tccgatcg

SEQ ID NO: 654 - DNA, 8 bp, Artificial Sequence
  Synthetic Oligonucleotide
tccgtacg

SEQ ID NO: 655 - DNA, 17 bp, Artificial Sequence
  Synthetic Oligonucleotide
gctagacgctagcgtga

SEQ ID NO: 656 - DNA, 25 bp, Artificial Sequence
  Synthetic Oligonucleotide
gagaacgctggacctcatcatccat

SEQ ID NO: 657 - DNA, 20 bp, Artificial Sequence
  Synthetic Oligonucleotide
gagaacgctagaccttctat

SEQ ID NO: 658 - DNA, 17 bp, Artificial Sequence
  Synthetic Oligonucleotide
actagacgttagtgtga

SEQ ID NO: 659 - DNA, 22 bp, Artificial Sequence
  Synthetic Oligonucleotide
cacaccttggtcaatgtcacgt

SEQ ID NO: 660 - DNA, 22 bp, Artificial Sequence
  Synthetic Oligonucleotide
tctccatcctatggttttatcg

SEQ ID NO: 661 - DNA, 15 bp, Artificial Sequence
  Synthetic Oligonucleotide
cgctggaccttccat

SEQ ID NO: 662 - DNA, 23 bp, Artificial Sequence
  Synthetic Oligonucleotide
caccaccttggtcaatgtcacgt

SEQ ID NO: 663 - DNA, 17 bp, Artificial Sequence
  Synthetic Oligonucleotide
gctagacgttagctgga

SEQ ID NO: 664 - DNA, 17 bp, Artificial Sequence
  Synthetic Oligonucleotide
agtgcgattgcagatcg

SEQ ID NO: 665 - DNA, 24 bp, Artificial Sequence
  Synthetic Oligonucleotide
ttttcgttttgtggttttgtggtt

SEQ ID NO: 666 - DNA, 23 bp, Artificial Sequence
  Synthetic Oligonucleotide
ttttcgtttgtcgttttgtcgtt

SEQ ID NO: 667 - DNA, 24 bp, Artificial Sequence
  Synthetic Oligonucleotide
tttttgttttgtggttttgtggtt

SEQ ID NO: 668 - DNA, 20 bp, Artificial Sequence
  Synthetic Oligonucleotide
accgcatggattctaggcca

SEQ ID NO: 669 - DNA, 15 bp, Artificial Sequence
  Synthetic Oligonucleotide
gctagacgttagcgt

SEQ ID NO: 670 - DNA, 17 bp, Artificial Sequence
  Synthetic Oligonucleotide
aacgctggaccttccat

SEQ ID NO: 671 - DNA, 8 bp, Artificial Sequence
  Synthetic Oligonucleotide
  modified_base (5)...(5): m5c
tcaacgtt

SEQ ID NO: 672 - DNA, 8 bp, Artificial Sequence
  Synthetic Oligonucleotide
ccttcgat

SEQ ID NO: 673 - DNA, 17 bp, Artificial Sequence
  Synthetic Oligonucleotide
actagacgttagtgtga

SEQ ID NO: 674 - DNA, 17 bp, Artificial Sequence
  Synthetic Oligonucleotide
gctagaggttagcgtga

SEQ ID NO: 675 - DNA, 20 bp, Artificial Sequence
  Synthetic Oligonucleotide
atggactctccagcgttctc

SEQ ID NO: 676 - DNA, 20 bp, Artificial Sequence
  Synthetic Oligonucleotide
atcgactctcgagcgttctc

SEQ ID NO: 677 - DNA, 13 bp, Artificial Sequence
  Synthetic Oligonucleotide
gctagacgttagc

SEQ ID NO: 678 - DNA, 9 bp, Artificial Sequence
  Synthetic Oligonucleotide
gctagacgt

SEQ ID NO: 679 - DNA, 17 bp, Artificial Sequence
  Synthetic Oligonucleotide
agtgcgattcgagatcg

SEQ ID NO: 680 - DNA, 8 bp, Artificial Sequence
  Synthetic Oligonucleotide
  modified_base (5)...(5): m5c
tcagcgct

SEQ ID NO: 681 - DNA, 18 bp, Artificial Sequence
  Synthetic Oligonucleotide
ctgattgctctctcgtga

SEQ ID NO: 682 - DNA, 8 bp, Artificial Sequence
  Synthetic Oligonucleotide
  modified_base (2)...(2): m5c
tcaacgtt

SEQ ID NO: 683 - DNA, 20 bp, Artificial Sequence
  Synthetic Oligonucleotide
  modified_base (6)...(6): m5c
gagaacgctggaccttccat

SEQ ID NO: 684 - DNA, 17 bp, Artificial Sequence
  Synthetic Oligonucleotide
gctagacgttaggctga

SEQ ID NO: 685 - DNA, 14 bp, Artificial Sequence
  Synthetic Oligonucleotide
gctacttagcgtga

SEQ ID NO: 686 - DNA, 15 bp, Artificial Sequence
  Synthetic Oligonucleotide
gctaccttagcgtga

SEQ ID NO: 687 - DNA, 19 bp, Artificial Sequence
  Synthetic Oligonucleotide
atcgacttcgagcgttctc

SEQ ID NO: 688 - DNA, 20 bp, Artificial Sequence
  Synthetic Oligonucleotide
atgcactctgcagcgttctc

SEQ ID NO: 689 - DNA, 20 bp, Artificial Sequence
  Synthetic Oligonucleotide
agtgactctccagcgttctc

SEQ ID NO: 690 - DNA, 17 bp, Artificial Sequence
  Synthetic Oligonucleotide
gccagatgttagctgga

SEQ ID NO: 691 - DNA, 18 bp, Artificial Sequence
  Synthetic Oligonucleotide
atcgactcgagcgttctc

SEQ ID NO: 692 - DNA, 17 bp, Artificial Sequence
  Synthetic Oligonucleotide
atcgatcgagcgttctc

SEQ ID NO: 693 - DNA, 20 bp, Artificial Sequence
  Synthetic Oligonucleotide
  misc_feature (1)...(1): biotinylated
gagaacgctcgaccttcgat

SEQ ID NO: 694 - DNA, 17 bp, Artificial Sequence
  Synthetic Oligonucleotide
gctagacgttagctgga

SEQ ID NO: 695 - DNA, 20 bp, Artificial Sequence
  Synthetic Oligonucleotide
atcgactctcgagcgttctc

SEQ ID NO: 696 - DNA, 15 bp, Artificial Sequence
  Synthetic Oligonucleotide
tagacgttagcgtga

SEQ ID NO: 697 - DNA, 18 bp, Artificial Sequence
  Synthetic Oligonucleotide
cgactctcgagcgttctc

SEQ ID NO: 698 - DNA, 21 bp, Artificial Sequence
  Synthetic Oligonucleotide
ggggtcgaccttggagggggg

SEQ ID NO: 699 - DNA, 16 bp, Artificial Sequence
  Synthetic Oligonucleotide
gctaacgttagcgtga

SEQ ID NO: 700 - DNA, 9 bp, Artificial Sequence
  Synthetic Oligonucleotide
cgtcgtcgt

SEQ ID NO: 701 - DNA, 20 bp, Artificial Sequence
  Synthetic Oligonucleotide
  modified_base (14)...(14): m5c
gagaacgctggaccttccat

SEQ ID NO: 702 - DNA, 20 bp, Artificial Sequence
  Synthetic Oligonucleotide
  modified_base (18)...(18): m5c
atcgacctacgtgcgttctc

SEQ ID NO: 703 - DNA, 20 bp, Artificial Sequence
  Synthetic Oligonucleotide
  modified_base (3)...(3): m5c
atcgacctacgtgcgttctc

SEQ ID NO: 704 - DNA, 15 bp, Artificial Sequence
  Synthetic Oligonucleotide
  modified_base (7)...(7): m5c
gctagacgttagcgt

SEQ ID NO: 705 - DNA, 20 bp, Artificial Sequence
  Synthetic Oligonucleotide
  modified_base (14)...(14): m5c
atcgactctcgagcgttctc

SEQ ID NO: 706 - DNA, 20 bp, Artificial Sequence
  Synthetic Oligonucleotide
ggggtaatgcatcagggggg

SEQ ID NO: 707 - DNA, 20 bp, Artificial Sequence
  Synthetic Oligonucleotide
ggctgtattcctgactgccc

SEQ ID NO: 708 - DNA, 17 bp, Artificial Sequence
  Synthetic Oligonucleotide
ccatgctaacctctagc

SEQ ID NO: 709 - DNA, 17 bp, Artificial Sequence
  Synthetic Oligonucleotide
gctagatgttagcgtga

SEQ ID NO: 710 - DNA, 15 bp, Artificial Sequence
  Synthetic Oligonucleotide
cgtaccttacggtga

SEQ ID NO: 711 - DNA, 20 bp, Artificial Sequence
  Synthetic Oligonucleotide
tccatgctggtcctgatgct

SEQ ID NO: 712 - DNA, 22 bp, Artificial Sequence
  Synthetic Oligonucleotide
atcgactctctcgagcgttctc

SEQ ID NO: 713 - DNA, 17 bp, Artificial Sequence
  Synthetic Oligonucleotide
gctagagcttagcgtga

SEQ ID NO: 714 - DNA, 20 bp, Artificial Sequence
  Synthetic Oligonucleotide
atcgactctcgagtgttctc

SEQ ID NO: 715 - DNA, 17 bp, Artificial Sequence
  Synthetic Oligonucleotide
aacgctcgaccttcgat

SEQ ID NO: 716 - DNA, 20 bp, Artificial Sequence
  Synthetic Oligonucleotide
ctcaacgctggaccttccat

SEQ ID NO: 717 - DNA, 20 bp, Artificial Sequence
  Synthetic Oligonucleotide
atcgacctacgtgcgttctc

SEQ ID NO: 718 - DNA, 20 bp, Artificial Sequence
  Synthetic Oligonucleotide
gagaatgctggaccttccat

SEQ ID NO: 719 - DNA, 17 bp, Artificial Sequence
  Synthetic Oligonucleotide
tcacgctaacctctgac

SEQ ID NO: 720 - DNA, 20 bp, Artificial Sequence
  Synthetic Oligonucleotide
  misc_feature (1)...(1): biotinylated
gagaacgctccagcactgat

SEQ ID NO: 721 - DNA, 20 bp, Artificial Sequence
  Synthetic Oligonucleotide
  misc_feature (1)...(1): biotinylated
gagcaagctggaccttccat

SEQ ID NO: 722 - DNA, 18 bp, Artificial Sequence
  Synthetic Oligonucleotide
cgctagaggttagcgtga

SEQ ID NO: 723 - DNA, 15 bp, Artificial Sequence
  Synthetic Oligonucleotide
gctagatgttaacgt

SEQ ID NO: 724 - DNA, 19 bp, Artificial Sequence
  Synthetic Oligonucleotide
atggaaggtccacgttctc

SEQ ID NO: 725 - DNA, 15 bp, Artificial Sequence
  Synthetic Oligonucleotide
gctagatgttagcgt

SEQ ID NO: 726 - DNA, 15 bp, Artificial Sequence
  Synthetic Oligonucleotide
gctagacgttagtgt

SEQ ID NO: 727 - DNA, 20 bp, Artificial Sequence
  Synthetic Oligonucleotide
tccatgacggtcctgatgct

SEQ ID NO: 728 - DNA, 20 bp, Artificial Sequence
  Synthetic Oligonucleotide
tccatggcggtcctgatgct

SEQ ID NO: 729 - DNA, 15 bp, Artificial Sequence
  Synthetic Oligonucleotide
gctagacgatagcgt

SEQ ID NO: 730 - DNA, 15 bp, Artificial Sequence
  Synthetic Oligonucleotide
gctagtcgatagcgt

SEQ ID NO: 731 - DNA, 20 bp, Artificial Sequence
  Synthetic Oligonucleotide
tccatgacgttcctgatgct

SEQ ID NO: 732 - DNA, 20 bp, Artificial Sequence
  Synthetic Oligonucleotide
tccatgtcgttcctgatgct

SEQ ID NO: 733 - DNA, 15 bp, Artificial Sequence
  Synthetic Oligonucleotide
  modified_base (13)...(13): m5c
gctagacgttagcgt

SEQ ID NO: 734 - DNA, 15 bp, Artificial Sequence
  Synthetic Oligonucleotide
gctaggcgttagcgt

SEQ ID NO: 735 - DNA, 20 bp, Artificial Sequence
  Synthetic Oligonucleotide
  modified_base (8)...(8): m5c
tccatgtcggtcctgatgct

SEQ ID NO: 736 - DNA, 20 bp, Artificial Sequence
  Synthetic Oligonucleotide
  modified_base (12)...(12): m5c
tccatgtcggtcctgatgct

SEQ ID NO: 737 - DNA, 20 bp, Artificial Sequence
  Synthetic Oligonucleotide
  modified_base (3)...(3): m5c
  modified_base (10)...(10): m5c
  modified_base (14)...(14): m5c
atcgactctcgagcgttctc

SEQ ID NO: 738 - DNA, 20 bp, Artificial Sequence
  Synthetic Oligonucleotide
atggaaggtccagtgttctc

SEQ ID NO: 739 - DNA, 15 bp, Artificial Sequence
  Synthetic Oligonucleotide
gcatgacgttgagct

SEQ ID NO: 740 - DNA, 20 bp, Artificial Sequence
  Synthetic Oligonucleotide
ggggtcaacgttgagggggg

SEQ ID NO: 741 - DNA, 20 bp, Artificial Sequence
  Synthetic Oligonucleotide
ggggtcaagtctgagggggg

SEQ ID NO: 742 - DNA, 20 bp, Artificial Sequence
  Synthetic Oligonucleotide
cgcgcgcgcgcgcgcgcgcg

SEQ ID NO: 743 - DNA, 28 bp, Artificial Sequence
  Synthetic Oligonucleotide
cccccccccccccccccccccccccccc

SEQ ID NO: 744 - DNA, 35 bp, Artificial Sequence
  Synthetic Oligonucleotide
ccccccccccccccccccccccccccccccccccc

SEQ ID NO: 745 - DNA, 20 bp, Artificial Sequence
  Synthetic Oligonucleotide
tccatgtcgctcctgatcct

SEQ ID NO: 746 - DNA, 15 bp, Artificial Sequence
  Synthetic Oligonucleotide
gctaaacgttagcgt

SEQ ID NO: 747 - DNA, 20 bp, Artificial Sequence
  Synthetic Oligonucleotide
tccatgtcgatcctgatgct

SEQ ID NO: 748 - DNA, 20 bp, Artificial Sequence
  Synthetic Oligonucleotide
tccatgccggtcctgatgct

SEQ ID NO: 749 - DNA, 20 bp, Artificial Sequence
  Synthetic Oligonucleotide
aaaatcaacgttgaaaaaaa

SEQ ID NO: 750 - DNA, 20 bp, Artificial Sequence
  Synthetic Oligonucleotide
tccataacgttcctgatgct

SEQ ID NO: 751 - DNA, 23 bp, Artificial Sequence
  Synthetic Oligonucleotide
tggaggtcccaccgagatcggag

SEQ ID NO: 752 - DNA, 21 bp, Artificial Sequence
  Synthetic Oligonucleotide
cgtcgtcgtcgtcgtcgtcgt

SEQ ID NO: 753 - DNA, 21 bp, Artificial Sequence
  Synthetic Oligonucleotide
ctgctgctgctgctgctgctg

SEQ ID NO: 754 - DNA, 21 bp, Artificial Sequence
  Synthetic Oligonucleotide
gagaacgctccgaccttcgat

SEQ ID NO: 755 - DNA, 15 bp, Artificial Sequence
  Synthetic Oligonucleotide
gctagatgttagcgt

SEQ ID NO: 756 - DNA, 15 bp, Artificial Sequence
  Synthetic Oligonucleotide
gcatgacgttgagct

SEQ ID NO: 757 - DNA, 10 bp, Artificial Sequence
  Synthetic Oligonucleotide
  misc_feature (10)...(10): fluorescein isothiocyanate conjugate
tcaatgctga

SEQ ID NO: 758 - DNA, 10 bp, Artificial Sequence
  Synthetic Oligonucleotide
  misc_feature (10)...(10): fluorescein isothiocyanate conjugate
tcaacgttga

SEQ ID NO: 759 - DNA, 10 bp, Artificial Sequence
  Synthetic Oligonucleotide
  misc_feature (10)...(10): biotinylated
tcaacgttga

SEQ ID NO: 760 - DNA, 10 bp, Artificial Sequence
  Synthetic Oligonucleotide
  misc_feature (10)...(10): biotinylated
gcaatattgc

SEQ ID NO: 761 - DNA, 10 bp, Artificial Sequence
  Synthetic Oligonucleotide
  misc_feature (10)...(10): fluorescein isothiocyanate conjugate
gcaatattgc

SEQ ID NO: 762 - DNA, 10 bp, Artificial Sequence
  Synthetic Oligonucleotide
agttgcaact

SEQ ID NO: 763 - DNA, 8 bp, Artificial Sequence
  Synthetic Oligonucleotide
tcttcgaa

SEQ ID NO: 764 - DNA, 8 bp, Artificial Sequence
  Synthetic Oligonucleotide
tcaacgtc

SEQ ID NO: 765 - DNA, 19 bp, Artificial Sequence
  Synthetic Oligonucleotide
ccatgtcggtcctgatgct

SEQ ID NO: 766 - DNA, 18 bp, Artificial Sequence
  Synthetic Oligonucleotide
gtttttatataatttggg

SEQ ID NO: 767 - DNA, 23 bp, Artificial Sequence
  Synthetic Oligonucleotide
tttttgtttgtcgttttgtcgtt

SEQ ID NO: 768 - DNA, 12 bp, Artificial Sequence
  Synthetic Oligonucleotide
ttggggggggtt

SEQ ID NO: 769 - DNA, 13 bp, Artificial Sequence
  Synthetic Oligonucleotide
ggggttgggggtt

SEQ ID NO: 770 - DNA, 17 bp, Artificial Sequence
  Synthetic Oligonucleotide
ggtggtgtaggttttgg

SEQ ID NO: 771 - DNA, 20 bp, Artificial Sequence
  Synthetic Oligonucleotide
  misc_feature (1)...(1): biotinylated
  modified_base (6)...(6): m5c
gagaacgctcgaccttcgat

SEQ ID NO: 772 - DNA, 20 bp, Artificial Sequence
  Synthetic Oligonucleotide
tcaacgttaacgttaacgtt

SEQ ID NO: 773 - DNA, 20 bp, Artificial Sequence
  Synthetic Oligonucleotide
  misc_feature (1)...(1): biotinylated
  modified_base (8)...(8): m5c
gagcaagctggaccttccat

SEQ ID NO: 774 - DNA, 20 bp, Artificial Sequence
  Synthetic Oligonucleotide
  misc_feature (1)...(1): biotinylated
  modified_base (6)...(6): m5c
gagaacgctccagcactgat

SEQ ID NO: 775 - DNA, 10 bp, Artificial Sequence
  Synthetic Oligonucleotide
  misc_feature (10)...(10): biotinylated
  modified_base (5)...(5): m5c
tcaacgttga

SEQ ID NO: 776 - DNA, 10 bp, Artificial Sequence
  Synthetic Oligonucleotide
  misc_feature (10)...(10): biotinylated
  modified_base (2)...(2): m5c
gcaatattgc

SEQ ID NO: 777 - DNA, 24 bp, Artificial Sequence
  Synthetic Oligonucleotide
tgctgcttttgtcgttttgtgctt

SEQ ID NO: 778 - DNA, 22 bp, Artificial Sequence
  Synthetic Oligonucleotide
ctgcgttagcaatttaactgtg

SEQ ID NO: 779 - DNA, 20 bp, Artificial Sequence
  Synthetic Oligonucleotide
tccatgacgttcctgatgct

SEQ ID NO: 780 - DNA, 28 bp, Artificial Sequence
  Synthetic Oligonucleotide
tgcatgccgtgcatccgtacacagctct

SEQ ID NO: 781 - DNA, 20 bp, Artificial Sequence
  Synthetic Oligonucleotide
tgcatgccgtacacagctct

SEQ ID NO: 782 - DNA, 12 bp, Artificial Sequence
  Synthetic Oligonucleotide
tgcatcagctct

SEQ ID NO: 783 - DNA, 8 bp, Artificial Sequence
  Synthetic Oligonucleotide
tgcgctct

SEQ ID NO: 784 - DNA, 12 bp, Artificial Sequence
  Synthetic Oligonucleotide
cccccccccccc

SEQ ID NO: 785 - DNA, 8 bp, Artificial Sequence
  Synthetic Oligonucleotide
cccccccc

SEQ ID NO: 786 - DNA, 12 bp, Artificial Sequence
  Synthetic Oligonucleotide
tgcatcagctct

SEQ ID NO: 787 - DNA, 20 bp, Artificial Sequence
  Synthetic Oligonucleotide
tgcatgccgtacacagctct

SEQ ID NO: 788 - DNA, 32 bp, Artificial Sequence
  Synthetic Oligonucleotide
tcaacgttaacgttaacgttaacgttaacgtt

SEQ ID NO: 789 - DNA, 20 bp, Artificial Sequence
  Synthetic Oligonucleotide
gagaacgctcgaccttcgat

SEQ ID NO: 790 - DNA, 25 bp, Artificial Sequence
  Synthetic Oligonucleotide
gtccccatttcccagaggaggaaat

SEQ ID NO: 791 - DNA, 25 bp, Artificial Sequence
  Synthetic Oligonucleotide
ctagcggctgacgtcatcaagctag

SEQ ID NO: 792 - DNA, 25 bp, Artificial Sequence
  Synthetic Oligonucleotide
ctagcttgatgacgtcagccgctag

SEQ ID NO: 793 - DNA, 16 bp, Artificial Sequence
  Synthetic Oligonucleotide
cggctgacgtcatcaa

SEQ ID NO: 794 - DNA, 8 bp, Artificial Sequence
  Synthetic Oligonucleotide
ctgacgtg

SEQ ID NO: 795 - DNA, 10 bp, Artificial Sequence
  Synthetic Oligonucleotide
ctgacgtcat

SEQ ID NO: 796 - DNA, 21 bp, Artificial Sequence
  Synthetic Oligonucleotide
attcgatcggggcggggcgag

SEQ ID NO: 797 - DNA, 21 bp, Artificial Sequence
  Synthetic Oligonucleotide
ctcgccccgccccgatcgaat

SEQ ID NO: 798 - DNA, 15 bp, Artificial Sequence
  Synthetic Oligonucleotide
gactgacgtcagcgt

SEQ ID NO: 799 - DNA, 26 bp, Artificial Sequence
  Synthetic Oligonucleotide
ctagcggctgacgtcataaagctagc

SEQ ID NO: 800 - DNA, 26 bp, Artificial Sequence
  Synthetic Oligonucleotide
ctagctttatgacgtcagccgctagc

SEQ ID NO: 801 - DNA, 26 bp, Artificial Sequence
  Synthetic Oligonucleotide
ctagcggctgagctcataaagctagc

SEQ ID NO: 802 - DNA, 25 bp, Artificial Sequence
  Synthetic Oligonucleotide
ctagtggctgacgtcatcaagctag

SEQ ID NO: 803 - DNA, 20 bp, Artificial Sequence
  Synthetic Oligonucleotide
tccaccacgtggtctatgct

SEQ ID NO: 804 - DNA, 24 bp, Artificial Sequence
  Synthetic Oligonucleotide
gggaatgaaagattttattataag

SEQ ID NO: 805 - DNA, 26 bp, Artificial Sequence
  Synthetic Oligonucleotide
tctaaaaaccatctattcttaaccct

SEQ ID NO: 806 - DNA, 15 bp, Artificial Sequence
  Synthetic Oligonucleotide
agctcaacgtcatgc

SEQ ID NO: 807 - DNA, 24 bp, Artificial Sequence
  Synthetic Oligonucleotide
ttaacggtggtagcggtattggtc

SEQ ID NO: 808 - DNA, 24 bp, Artificial Sequence
  Synthetic Oligonucleotide
ttaagaccaataccgctaccaccg

SEQ ID NO: 809 - DNA, 25 bp, Artificial Sequence
  Synthetic Oligonucleotide
gatctagtgatgagtcagccggatc

SEQ ID NO: 810 - DNA, 25 bp, Artificial Sequence
  Synthetic Oligonucleotide
gatccggctgactcatcactagatc

SEQ ID NO: 811 - DNA, 20 bp, Artificial Sequence
  Synthetic Oligonucleotide
tccaagacgttcctgatgct

SEQ ID NO: 812 - DNA, 20 bp, Artificial Sequence
  Synthetic Oligonucleotide
tccatgacgtccctgatgct

SEQ ID NO: 813 - DNA, 20 bp, Artificial Sequence
  Synthetic Oligonucleotide
tccaccacgtggctgatgct

SEQ ID NO: 814 - DNA, 17 bp, Artificial Sequence
  Synthetic Oligonucleotide
ccacgtggacctctagc

SEQ ID NO: 815 - DNA, 27 bp, Artificial Sequence
  Synthetic Oligonucleotide
tcagaccacgtggtcgggtgttcctga

SEQ ID NO: 816 - DNA, 27 bp, Artificial Sequence
  Synthetic Oligonucleotide
tcaggaacacccgaccacgtggtctga

SEQ ID NO: 817 - DNA, 18 bp, Artificial Sequence
  Synthetic Oligonucleotide
catttccacgatttccca

SEQ ID NO: 818 - DNA, 19 bp, Artificial Sequence
  Synthetic Oligonucleotide
ttcctctctgcaagagact

SEQ ID NO: 819 - DNA, 19 bp, Artificial Sequence
  Synthetic Oligonucleotide
tgtatctctctgaaggact

SEQ ID NO: 820 - DNA, 25 bp, Artificial Sequence
  Synthetic Oligonucleotide
ataaagcgaaactagcagcagtttc

SEQ ID NO: 821 - DNA, 25 bp, Artificial Sequence
  Synthetic Oligonucleotide
gaaactgctgctagtttcgctttat

SEQ ID NO: 822 - DNA, 30 bp, Artificial Sequence
  Synthetic Oligonucleotide
tgcccaaagaggaaaatttgtttcatacag

SEQ ID NO: 823 - DNA, 30 bp, Artificial Sequence
  Synthetic Oligonucleotide
ctgtatgaaacaaattttcctctttgggca

SEQ ID NO: 824 - DNA, 20 bp, Artificial Sequence
  Synthetic Oligonucleotide
ttagggttagggttagggtt

SEQ ID NO: 825 - DNA, 20 bp, Artificial Sequence
  Synthetic Oligonucleotide
tccatgagcttcctgatgct

SEQ ID NO: 826 - DNA, 20 bp, Artificial Sequence
  Synthetic Oligonucleotide
aaaacatgacgttcaaaaaa

SEQ ID NO: 827 - DNA, 20 bp, Artificial Sequence
  Synthetic Oligonucleotide
aaaacatgacgttcgggggg

SEQ ID NO: 828 - DNA, 20 bp, Artificial Sequence
  Synthetic Oligonucleotide
ggggcatgagcttcgggggg

SEQ ID NO: 829 - DNA, 24 bp, Artificial Sequence
  Synthetic Oligonucleotide
ctaggctgacgtcatcaagctagt

SEQ ID NO: 830 - DNA, 30 bp, Artificial Sequence
  Synthetic Oligonucleotide
tctgacgtcatctgacgttggctgacgtct

SEQ ID NO: 831 - DNA, 25 bp, Artificial Sequence
  Synthetic Oligonucleotide
ggaattagtaatagatatagaagtt

SEQ ID NO: 832 - DNA, 30 bp, Artificial Sequence
  Synthetic Oligonucleotide
tttaccttttataaacataactaaaacaaa

SEQ ID NO: 833 - DNA, 15 bp, Artificial Sequence
  Synthetic Oligonucleotide
gcgtttttttttgcg

SEQ ID NO: 834 - DNA, 24 bp, Artificial Sequence
  Synthetic Oligonucleotide
atatctaatcaaaacattaacaaa

SEQ ID NO: 835 - DNA, 24 bp, Artificial Sequence
  Synthetic Oligonucleotide
tctatcccaggtggttcctgttag

SEQ ID NO: 836 - DNA, 20 bp, Artificial Sequence
  Synthetic Oligonucleotide
  misc_feature (1)...(1): biotinylated
tccatgacgttcctgatgct

SEQ ID NO: 837 - DNA, 20 bp, Artificial Sequence
  Synthetic Oligonucleotide
  misc_feature (1)...(1): biotinylated
tccatgagcttcctgatgct

SEQ ID NO: 838 - DNA, 13 bp, Artificial Sequence
  Synthetic Oligonucleotide
  misc_feature (13)...(13): fluorescein isothiocyanate conjugate
ttttttttttttt

SEQ ID NO: 839 - DNA, 13 bp, Artificial Sequence
  Synthetic Oligonucleotide
  misc_feature (13)...(13): fluorescein isothiocyanate conjugate
ttttttttttttt

SEQ ID NO: 840 - DNA, 25 bp, Artificial Sequence
  Synthetic Oligonucleotide
ctagcttgatgagctcagccgctag

SEQ ID NO: 841 - DNA, 25 bp, Artificial Sequence
  Synthetic Oligonucleotide
ttcagttgtcttgctgcttagctaa

SEQ ID NO: 842 - DNA, 20 bp, Artificial Sequence
  Synthetic Oligonucleotide
tccatgagcttcctgagtct

SEQ ID NO: 843 - DNA, 25 bp, Artificial Sequence
  Synthetic Oligonucleotide
ctagcggctgacgtcatcaatctag

SEQ ID NO: 844 - DNA, 20 bp, Artificial Sequence
  Synthetic Oligonucleotide
tgctagctgtgcctgtacct

SEQ ID NO: 845 - DNA, 23 bp, Artificial Sequence
  Synthetic Oligonucleotide
atgctaaaggacgtcacattgca

SEQ ID NO: 846 - DNA, 23 bp, Artificial Sequence
  Synthetic Oligonucleotide
tgcaatgtgacgtcctttagcat

SEQ ID NO: 847 - DNA, 31 bp, Artificial Sequence
  Synthetic Oligonucleotide
gtaggggactttccgagctcgagatcctatg

SEQ ID NO: 848 - DNA, 31 bp, Artificial Sequence
  Synthetic Oligonucleotide
cataggatctcgagctcggaaagtcccctac

SEQ ID NO: 849 - DNA, 22 bp, Artificial Sequence
  Synthetic Oligonucleotide
ctgtcaggaactgcaggtaagg

SEQ ID NO: 850 - DNA, 27 bp, Artificial Sequence
  Synthetic Oligonucleotide
cataacataggaatatttactcctcgc

SEQ ID NO: 851 - DNA, 21 bp, Artificial Sequence
  Synthetic Oligonucleotide
ctccagctccaagaaaggacg

SEQ ID NO: 852 - DNA, 21 bp, Artificial Sequence
  Synthetic Oligonucleotide
gaagtttctggtaagtcttcg

SEQ ID NO: 853 - DNA, 24 bp, Artificial Sequence
  Synthetic Oligonucleotide
tgctgcttttgtgcttttgtgctt

SEQ ID NO: 854 - DNA, 24 bp, Artificial Sequence
  Synthetic Oligonucleotide
tcgtcgttttgtggttttgtggtt

SEQ ID NO: 855 - DNA, 23 bp, Artificial Sequence
  Synthetic Oligonucleotide
tcgtcgtttgtcgttttgtcgtt

SEQ ID NO: 856 - DNA, 22 bp, Artificial Sequence
  Synthetic Oligonucleotide
tcctgacgttcggcgcgcgccc

SEQ ID NO: 857 - DNA, 24 bp, Artificial Sequence
  Synthetic Oligonucleotide
tgctgcttttgtgcttttgtgctt

SEQ ID NO: 858 - DNA, 20 bp, Artificial Sequence
  Synthetic Oligonucleotide
tccatgagcttcctgagctt

SEQ ID NO: 859 - DNA, 24 bp, Artificial Sequence
  Synthetic Oligonucleotide
tcgtcgtttcgtcgttttgacgtt

SEQ ID NO: 860 - DNA, 26 bp, Artificial Sequence
  Synthetic Oligonucleotide
tcgtcgtttgcgtgcgtttcgtcgtt

SEQ ID NO: 861 - DNA, 27 bp, Artificial Sequence
  Synthetic Oligonucleotide
tcgcgtgcgttttgtcgttttgacgtt

SEQ ID NO: 862 - DNA, 25 bp, Artificial Sequence
  Synthetic Oligonucleotide
ttcgtcgttttgtcgttttgtcgtt

SEQ ID NO: 863 - DNA, 15 bp, Artificial Sequence
  Synthetic Oligonucleotide
tcctgacggggaagt

SEQ ID NO: 864 - DNA, 15 bp, Artificial Sequence
  Synthetic Oligonucleotide
tcctggcggtgaagt

SEQ ID NO: 865 - DNA, 15 bp, Artificial Sequence
  Synthetic Oligonucleotide
tcctggcgttgaagt

SEQ ID NO: 866 - DNA, 15 bp, Artificial Sequence
  Synthetic Oligonucleotide
tcctgacgtggaagt

SEQ ID NO: 867 - DNA, 20 bp, Artificial Sequence
  Synthetic Oligonucleotide
gcgacgttcggcgcgcgccc

SEQ ID NO: 868 - DNA, 20 bp, Artificial Sequence
  Synthetic Oligonucleotide
gcgacgggcggcgcgcgccc

SEQ ID NO: 869 - DNA, 20 bp, Artificial Sequence
  Synthetic Oligonucleotide
gcggcgtgcggcgcgcgccc

SEQ ID NO: 870 - DNA, 20 bp, Artificial Sequence
  Synthetic Oligonucleotide
gcggcggtcggcgcgcgccc

SEQ ID NO: 871 - DNA, 20 bp, Artificial Sequence
  Synthetic Oligonucleotide
gcgacggtcggcgcgcgccc

SEQ ID NO: 872 - DNA, 20 bp, Artificial Sequence
  Synthetic Oligonucleotide
gcggcgttcggcgcgcgccc

SEQ ID NO: 873 - DNA, 20 bp, Artificial Sequence
  Synthetic Oligonucleotide
gcgacgtgcggcgcgcgccc

SEQ ID NO: 874 - DNA, 20 bp, Artificial Sequence
  Synthetic Oligonucleotide
tgtgggggttttggttttgg

SEQ ID NO: 875 - DNA, 20 bp, Artificial Sequence
  Synthetic Oligonucleotide
aggggaggggaggggagggg

SEQ ID NO: 876 - DNA, 21 bp, Artificial Sequence
  Synthetic Oligonucleotide
tgtgtgtgtgtgtgtgtgtgt

SEQ ID NO: 877 - DNA, 22 bp, Artificial Sequence
  Synthetic Oligonucleotide
ctctctctctctctctctctct

SEQ ID NO: 878 - DNA, 20 bp, Artificial Sequence
  Synthetic Oligonucleotide
ggggtcgacgtcgagggggg

SEQ ID NO: 879 - DNA, 22 bp, Artificial Sequence
  Synthetic Oligonucleotide
atatatatatatatatatatat

SEQ ID NO: 880 - DNA, 27 bp, Artificial Sequence
  Synthetic Oligonucleotide
ttttttttttttttttttttttttttt

SEQ ID NO: 881 - DNA, 21 bp, Artificial Sequence
  Synthetic Oligonucleotide
ttttttttttttttttttttt

SEQ ID NO: 882 - DNA, 18 bp, Artificial Sequence
  Synthetic Oligonucleotide
tttttttttttttttttt

SEQ ID NO: 883 - DNA, 15 bp, Artificial Sequence
  Synthetic Oligonucleotide
gctagaggggagggt

SEQ ID NO: 884 - DNA, 15 bp, Artificial Sequence
  Synthetic Oligonucleotide
gctagatgttagggg

SEQ ID NO: 885 - DNA, 15 bp, Artificial Sequence
  Synthetic Oligonucleotide
gcatgagggggagct

SEQ ID NO: 886 - DNA, 20 bp, Artificial Sequence
  Synthetic Oligonucleotide
atggaaggtccagggggctc

SEQ ID NO: 887 - DNA, 20 bp, Artificial Sequence
  Synthetic Oligonucleotide
atggactctggagggggctc

SEQ ID NO: 888 - DNA, 20 bp, Artificial Sequence
  Synthetic Oligonucleotide
atggaaggtccaaggggctc

SEQ ID NO: 889 - DNA, 20 bp, Artificial Sequence
  Synthetic Oligonucleotide
gagaaggggggaccttggat

SEQ ID NO: 890 - DNA, 20 bp, Artificial Sequence
  Synthetic Oligonucleotide
gagaaggggggaccttccat

SEQ ID NO: 891 - DNA, 20 bp, Artificial Sequence
  Synthetic Oligonucleotide
gagaaggggccagcactgat

SEQ ID NO: 892 - DNA, 20 bp, Artificial Sequence
  Synthetic Oligonucleotide
tccatgtggggcctgatgct

SEQ ID NO: 893 - DNA, 20 bp, Artificial Sequence
  Synthetic Oligonucleotide
tccatgaggggcctgatgct

SEQ ID NO: 894 - DNA, 20 bp, Artificial Sequence
  Synthetic Oligonucleotide
tccatgtggggcctgctgat

SEQ ID NO: 895 - DNA, 20 bp, Artificial Sequence
  Synthetic Oligonucleotide
atggactctccggggttctc

SEQ ID NO: 896 - DNA, 20 bp, Artificial Sequence
  Synthetic Oligonucleotide
atggaaggtccggggttctc

SEQ ID NO: 897 - DNA, 20 bp, Artificial Sequence
  Synthetic Oligonucleotide
atggactctggaggggtctc

SEQ ID NO: 898 - DNA, 20 bp, Artificial Sequence
  Synthetic Oligonucleotide
atggaggctccatggggctc

SEQ ID NO: 899 - DNA, 20 bp, Artificial Sequence
  Synthetic Oligonucleotide
atggactctggggggttctc

SEQ ID NO: 900 - DNA, 20 bp, Artificial Sequence
  Synthetic Oligonucleotide
tccatgtgggtggggatgct

SEQ ID NO: 901 - DNA, 20 bp, Artificial Sequence
  Synthetic Oligonucleotide
tccatgcgggtggggatgct

SEQ ID NO: 902 - DNA, 20 bp, Artificial Sequence
  Synthetic Oligonucleotide
tccatgggggtcctgatgct

SEQ ID NO: 903 - DNA, 20 bp, Artificial Sequence
  Synthetic Oligonucleotide
tccatggggtccctgatgct

SEQ ID NO: 904 - DNA, 20 bp, Artificial Sequence
  Synthetic Oligonucleotide
tccatggggtgcctgatgct

SEQ ID NO: 905 - DNA, 20 bp, Artificial Sequence
  Synthetic Oligonucleotide
tccatggggttcctgatgct

SEQ ID NO: 906 - DNA, 20 bp, Artificial Sequence
  Synthetic Oligonucleotide
tccatcgggggcctgatgct

SEQ ID NO: 907 - DNA, 14 bp, Artificial Sequence
  Synthetic Oligonucleotide
gctagagggagtgt

SEQ ID NO: 908 - DNA, 21 bp, Artificial Sequence
  Synthetic Oligonucleotide
gmggtcaacgttgagggmggg

SEQ ID NO: 909 - DNA, 21 bp, Artificial Sequence
  Synthetic Oligonucleotide
ggggagttcgttgaggggggg

SEQ ID NO: 910 - DNA, 20 bp, Artificial Sequence
  Synthetic Oligonucleotide
tcgtcgtttccccccccccc

SEQ ID NO: 911 - DNA, 25 bp, Artificial Sequence
  Synthetic Oligonucleotide
ttggggggttttttttttttttttt

SEQ ID NO: 912 - DNA, 23 bp, Artificial Sequence
  Synthetic Oligonucleotide
tttaaattttaaaatttaaaata

SEQ ID NO: 913 - DNA, 24 bp, Artificial Sequence
  Synthetic Oligonucleotide
ttggtttttttggtttttttttgg

SEQ ID NO: 914 - DNA, 24 bp, Artificial Sequence
  Synthetic Oligonucleotide
tttcccttttccccttttcccctc

SEQ ID NO: 915 - DNA, 21 bp, Artificial Sequence
  Synthetic Oligonucleotide
ggggtcatcgatgaggggggs

SEQ ID NO: 916 - DNA, 20 bp, Artificial Sequence
  Synthetic Oligonucleotide
tccatgacgttcctgacgtt

SEQ ID NO: 917 - DNA, 19 bp, Artificial Sequence
  Synthetic Oligonucleotide
gggggacgatcgtcggggg

SEQ ID NO: 918 - DNA, 20 bp, Artificial Sequence
  Synthetic Oligonucleotide
gggggtcgtacgacgggggg

SEQ ID NO: 919 - DNA, 24 bp, Artificial Sequence
  Synthetic Oligonucleotide
tttttttttttttttttttttttt

SEQ ID NO: 920 - DNA, 24 bp, Artificial Sequence
  Synthetic Oligonucleotide
aaaaaaaaaaaaaaaaaaaaaaaa

SEQ ID NO: 921 - DNA, 24 bp, Artificial Sequence
  Synthetic Oligonucleotide
cccccccccccccccccccccccc

SEQ ID NO: 922 - DNA, 24 bp, Artificial Sequence
  Synthetic Oligonucleotide
tcgtcgttttgtcgttttgtcgtt

SEQ ID NO: 923 - DNA, 20 bp, Artificial Sequence
  Synthetic Oligonucleotide
ggggtcaacgttgagggggg

SEQ ID NO: 924 - DNA, 20 bp, Artificial Sequence
  Synthetic Oligonucleotide
ggggtcaagcttgagggggg

SEQ ID NO: 925 - DNA, 20 bp, Artificial Sequence
  Synthetic Oligonucleotide
tgctgcttcccccccccccc

SEQ ID NO: 926 - DNA, 20 bp, Artificial Sequence
  Synthetic Oligonucleotide
ggggacgtcgacgtgggggg

SEQ ID NO: 927 - DNA, 20 bp, Artificial Sequence
  Synthetic Oligonucleotide
ggggtcgtcgacgagggggg

SEQ ID NO: 928 - DNA, 24 bp, Artificial Sequence
  Synthetic Oligonucleotide
ggggtcgacgtacgtcgagggggg

SEQ ID NO: 929 - DNA, 22 bp, Artificial Sequence
  Synthetic Oligonucleotide
ggggaccggtaccggtgggggg

SEQ ID NO: 930 - DNA, 19 bp, Artificial Sequence
  Synthetic Oligonucleotide
gggtcgacgtcgagggggg

SEQ ID NO: 931 - DNA, 19 bp, Artificial Sequence
  Synthetic Oligonucleotide
ggggtcgacgtcgaggggg

SEQ ID NO: 932 - DNA, 22 bp, Artificial Sequence
  Synthetic Oligonucleotide
ggggaacgttaacgttgggggg

SEQ ID NO: 933 - DNA, 20 bp, Artificial Sequence
  Synthetic Oligonucleotide
ggggtcaccggtgagggggg

SEQ ID NO: 934 - DNA, 22 bp, Artificial Sequence
  Synthetic Oligonucleotide
ggggtcgttcgaacgagggggg

SEQ ID NO: 935 - DNA, 22 bp, Artificial Sequence
  Synthetic Oligonucleotide
ggggacgttcgaacgtgggggg

SEQ ID NO: 936 - DNA, 10 bp, Artificial Sequence
  Synthetic Oligonucleotide
tcaactttga

SEQ ID NO: 937 - DNA, 10 bp, Artificial Sequence
  Synthetic Oligonucleotide
tcaagcttga

SEQ ID NO: 938 - DNA, 12 bp, Artificial Sequence
  Synthetic Oligonucleotide
tcacgatcgtga

SEQ ID NO: 939 - DNA, 12 bp, Artificial Sequence
  Synthetic Oligonucleotide
tcagcatgctga

SEQ ID NO: 940 - DNA, 20 bp, Artificial Sequence
  Synthetic Oligonucleotide
gggggagcatgctggggggg

SEQ ID NO: 941 - DNA, 22 bp, Artificial Sequence
  Synthetic Oligonucleotide
gggggacgatatcgtcgggggg

SEQ ID NO: 942 - DNA, 22 bp, Artificial Sequence
  Synthetic Oligonucleotide
gggggacgacgtcgtcgggggg

SEQ ID NO: 943 - DNA, 22 bp, Artificial Sequence
  Synthetic Oligonucleotide
gggggacgagctcgtcgggggg

SEQ ID NO: 944 - DNA, 20 bp, Artificial Sequence
  Synthetic Oligonucleotide
gggggacgtacgtcgggggg

SEQ ID NO: 945 - DNA, 8 bp, Artificial Sequence
  Synthetic Oligonucleotide
tcaacgtt

SEQ ID NO: 946 - DNA, 20 bp, Artificial Sequence
  Synthetic Oligonucleotide
tccataccggtcctgatgct

SEQ ID NO: 947 - DNA, 20 bp, Artificial Sequence
  Synthetic Oligonucleotide
tccataccggtcctaccggt

SEQ ID NO: 948 - DNA, 20 bp, Artificial Sequence
  Synthetic Oligonucleotide
gggggacgatcgttgggggg

SEQ ID NO: 949 - DNA, 20 bp, Artificial Sequence
  Synthetic Oligonucleotide
ggggaacgatcgtcgggggg

SEQ ID NO: 950 - DNA, 21 bp, Artificial Sequence
  Synthetic Oligonucleotide
ggggggacgatcgtcgggggg

SEQ ID NO: 951 - DNA, 21 bp, Artificial Sequence
  Synthetic Oligonucleotide
gggggacgatcgtcggggggg

SEQ ID NO: 952 - DNA, 12 bp, Artificial Sequence
  Synthetic Oligonucleotide
aaagacgttaaa

SEQ ID NO: 953 - DNA, 12 bp, Artificial Sequence
  Synthetic Oligonucleotide
aaagagcttaaa

SEQ ID NO: 954 - DNA, 12 bp, Artificial Sequence
  Synthetic Oligonucleotide
  modified_base (6)...(6): m5c
aaagacgttaaa

SEQ ID NO: 955 - DNA, 12 bp, Artificial Sequence
  Synthetic Oligonucleotide
aaattcggaaaa

SEQ ID NO: 956 - DNA, 21 bp, Artificial Sequence
  Synthetic Oligonucleotide
gggggtcatcgatgagggggg

SEQ ID NO: 957 - DNA, 21 bp, Artificial Sequence
  Synthetic Oligonucleotide
gggggtcaacgttgagggggg

SEQ ID NO: 958 - DNA, 20 bp, Artificial Sequence
  Synthetic Oligonucleotide
atgtagcttaataacaaagc

SEQ ID NO: 959 - DNA, 20 bp, Artificial Sequence
  Synthetic Oligonucleotide
ggatcccttgagttacttct

SEQ ID NO: 960 - DNA, 20 bp, Artificial Sequence
  Synthetic Oligonucleotide
ccattccacttctgattacc

SEQ ID NO: 961 - DNA, 20 bp, Artificial Sequence
  Synthetic Oligonucleotide
tatgtattatcatgtagata

SEQ ID NO: 962 - DNA, 20 bp, Artificial Sequence
  Synthetic Oligonucleotide
agcctacgtattcaccctcc

SEQ ID NO: 963 - DNA, 20 bp, Artificial Sequence
  Synthetic Oligonucleotide
ttcctgcaactactattgta

SEQ ID NO: 964 - DNA, 20 bp, Artificial Sequence
  Synthetic Oligonucleotide
atagaaggccctacaccagt

SEQ ID NO: 965 - DNA, 20 bp, Artificial Sequence
  Synthetic Oligonucleotide
ttacaccggtctatggaggt

SEQ ID NO: 966 - DNA, 20 bp, Artificial Sequence
  Synthetic Oligonucleotide
ctaaccagatcaagtctagg

SEQ ID NO: 967 - DNA, 20 bp, Artificial Sequence
  Synthetic Oligonucleotide
cctagacttgatctggttag

SEQ ID NO: 968 - DNA, 20 bp, Artificial Sequence
  Synthetic Oligonucleotide
tataagcctcgtccgacatg

SEQ ID NO: 969 - DNA, 20 bp, Artificial Sequence
  Synthetic Oligonucleotide
catgtcggacgaggcttata

SEQ ID NO: 970 - DNA, 20 bp, Artificial Sequence
  Synthetic Oligonucleotide
tggtggtggggagtaagctc

SEQ ID NO: 971 - DNA, 20 bp, Artificial Sequence
  Synthetic Oligonucleotide
gagctactcccccaccacca

SEQ ID NO: 972 - DNA, 20 bp, Artificial Sequence
  Synthetic Oligonucleotide
gccttcgatcttcgttggga

SEQ ID NO: 973 - DNA, 20 bp, Artificial Sequence
  Synthetic Oligonucleotide
tggacttctctttgccgtct

SEQ ID NO: 974 - DNA, 20 bp, Artificial Sequence
  Synthetic Oligonucleotide
atgctgtagcccagcgataa

SEQ ID NO: 975 - DNA, 20 bp, Artificial Sequence
  Synthetic Oligonucleotide
accgaatcagcggaaagtga

SEQ ID NO: 976 - DNA, 24 bp, Artificial Sequence
  Synthetic Oligonucleotide
ggagaaacccatgagctcatctgg

SEQ ID NO: 977 - DNA, 20 bp, Artificial Sequence
  Synthetic Oligonucleotide
accacagaccagcaggcaga

SEQ ID NO: 978 - DNA, 20 bp, Artificial Sequence
  Synthetic Oligonucleotide
gagcgtgaactgcgcgaaga

SEQ ID NO: 979 - DNA, 20 bp, Artificial Sequence
  Synthetic Oligonucleotide
tcggtacccttgcagcggtt

SEQ ID NO: 980 - DNA, 20 bp, Artificial Sequence
  Synthetic Oligonucleotide
ctggagccctagccaaggat

SEQ ID NO: 981 - DNA, 20 bp, Artificial Sequence
  Synthetic Oligonucleotide
gcgactccatcaccagcgat

SEQ ID NO: 982 - DNA, 21 bp, Artificial Sequence
  Synthetic Oligonucleotide
cctgaagtaagaaccagatgt

SEQ ID NO: 983 - DNA, 21 bp, Artificial Sequence
  Synthetic Oligonucleotide
ctgtgttatctgacatacacc

SEQ ID NO: 984 - DNA, 21 bp, Artificial Sequence
  Synthetic Oligonucleotide
aattagccttaggtgattggg

SEQ ID NO: 985 - DNA, 21 bp, Artificial Sequence
  Synthetic Oligonucleotide
acatctggttcttacttcagg

SEQ ID NO: 986 - DNA, 23 bp, Artificial Sequence
  Synthetic Oligonucleotide
ataagtcatattttgggaactac

SEQ ID NO: 987 - DNA, 21 bp, Artificial Sequence
  Synthetic Oligonucleotide
cccaatcacctaaggctaatt

SEQ ID NO: 988 - DNA, 15 bp, Artificial Sequence
  Synthetic Oligonucleotide
  modified_base (9)...(9): m5c
tcctggcgcggaagt

SEQ ID NO: 989 - DNA, 22 bp, Artificial Sequence
  Synthetic Oligonucleotide
ggggaacgacgtcgttgggggg

SEQ ID NO: 990 - DNA, 20 bp, Artificial Sequence
  Synthetic Oligonucleotide
ggggaacgtacgtcgggggg

SEQ ID NO: 991 - DNA, 24 bp, Artificial Sequence
  Synthetic Oligonucleotide
ggggaacgtacgtacgttgggggg

SEQ ID NO: 992 - DNA, 18 bp, Artificial Sequence
  Synthetic Oligonucleotide
ggggtcgacgtcgagggg

SEQ ID NO: 993 - DNA, 19 bp, Artificial Sequence
  Synthetic Oligonucleotide
ggggacgtcgacgtggggg

SEQ ID NO: 994 - DNA, 34 bp, Artificial Sequence
  Synthetic Oligonucleotide
gcactcttcgaagctacagccggcagcctctgat

SEQ ID NO: 995 - DNA, 32 bp, Artificial Sequence
  Synthetic Oligonucleotide
cggctcttccatgaggtctttgctaatcttgg

SEQ ID NO: 996 - DNA, 35 bp, Artificial Sequence
  Synthetic Oligonucleotide
cggctcttccatgaaagtctttggacgatgtgagc

SEQ ID NO: 997 - DNA, 15 bp, Artificial Sequence
  Synthetic Oligonucleotide
tcctgcaggttaagt

SEQ ID NO: 998 - DNA, 20 bp, Artificial Sequence
  Synthetic Oligonucleotide
gggggtcgttcgttgggggg

SEQ ID NO: 999 - DNA, 20 bp, Artificial Sequence
  Synthetic Oligonucleotide
gggggatgattgttgggggg

SEQ ID NO: 1000 - DNA, 20 bp, Artificial Sequence
  Synthetic Oligonucleotide
  modified_base (7)...(7): m5c
  modified_base (11)...(11): m5c
gggggacgatcgttgggggg

SEQ ID NO: 1001 - DNA, 20 bp, Artificial Sequence
  Synthetic Oligonucleotide
gggggagctagcttgggggg

SEQ ID NO: 1002 - DNA, 20 bp, Artificial Sequence
  Synthetic Oligonucleotide
ggttcttttggtccttgtct

SEQ ID NO: 1003 - DNA, 20 bp, Artificial Sequence
  Synthetic Oligonucleotide
ggttcttttggtcctcgtct

SEQ ID NO: 1004 - DNA, 20 bp, Artificial Sequence
  Synthetic Oligonucleotide
ggttcttttggtccttatct

SEQ ID NO: 1005 - DNA, 20 bp, Artificial Sequence
  Synthetic Oligonucleotide
ggttcttggtttccttgtct

SEQ ID NO: 1006 - DNA, 20 bp, Artificial Sequence
  Synthetic Oligonucleotide
tggtcttttggtccttgtct

SEQ ID NO: 1007 - DNA, 20 bp, Artificial Sequence
  Synthetic Oligonucleotide
ggttcaaatggtccttgtct

SEQ ID NO: 1008 - DNA, 20 bp, Artificial Sequence
  Synthetic Oligonucleotide
gggtcttttgggccttgtct

SEQ ID NO: 1009 - DNA, 24 bp, Artificial Sequence
  Synthetic Oligonucleotide
tccaggacttctctcaggtttttt

SEQ ID NO: 1010 - DNA, 20 bp, Artificial Sequence
  Synthetic Oligonucleotide
tccaaaacttctctcaaatt

SEQ ID NO: 1011 - DNA, 24 bp, Artificial Sequence
  Synthetic Oligonucleotide
tactacttttatacttttatactt

SEQ ID NO: 1012 - DNA, 24 bp, Artificial Sequence
  Synthetic Oligonucleotide
tgtgtgtgtgtgtgtgtgtgtgtg

SEQ ID NO: 1013 - DNA, 25 bp, Artificial Sequence
  Synthetic Oligonucleotide
ttgttgttgttgtttgttgttgttg

SEQ ID NO: 1014 - DNA, 27 bp, Artificial Sequence
  Synthetic Oligonucleotide
ggctccggggagggaatttttgtctat

SEQ ID NO: 1015 - DNA, 19 bp, Artificial Sequence
  Synthetic Oligonucleotide
gggacgatcgtcggggggg

SEQ ID NO: 1016 - DNA, 20 bp, Artificial Sequence
  Synthetic Oligonucleotide
gggtcgtcgacgaggggggg

SEQ ID NO: 1017 - DNA, 19 bp, Artificial Sequence
  Synthetic Oligonucleotide
ggtcgtcgacgaggggggg

SEQ ID NO: 1018 - DNA, 20 bp, Artificial Sequence
  Synthetic Oligonucleotide
gggtcgtcgtcgtggggggg

SEQ ID NO: 1019 - DNA, 20 bp, Artificial Sequence
  Synthetic Oligonucleotide
ggggacgatcgtcggggggg

SEQ ID NO: 1020 - DNA, 20 bp, Artificial Sequence
  Synthetic Oligonucleotide
ggggacgtcgtcgtgggggg

SEQ ID NO: 1021 - DNA, 27 bp, Artificial Sequence
  Synthetic Oligonucleotide
ggggtcgacgtcgacgtcgaggggggg

SEQ ID NO: 1022 - DNA, 21 bp, Artificial Sequence
  Synthetic Oligonucleotide
ggggaaccgcggttggggggg

SEQ ID NO: 1023 - DNA, 21 bp, Artificial Sequence
  Synthetic Oligonucleotide
ggggacgacgtcgtggggggg

SEQ ID NO: 1024 - DNA, 23 bp, Artificial Sequence
  Synthetic Oligonucleotide
tcgtcgtcgtcgtcgtggggggg

SEQ ID NO: 1025 - DNA, 15 bp, Artificial Sequence
  Synthetic Oligonucleotide
tcctgccggggaagt

SEQ ID NO: 1026 - DNA, 15 bp, Artificial Sequence
  Synthetic Oligonucleotide
tcctgcaggggaagt

SEQ ID NO: 1027 - DNA, 15 bp, Artificial Sequence
  Synthetic Oligonucleotide
tcctgaaggggaagt

SEQ ID NO: 1028 - DNA, 15 bp, Artificial Sequence
  Synthetic Oligonucleotide
tcctggcgggcaagt

SEQ ID NO: 1029 - DNA, 15 bp, Artificial Sequence
  Synthetic Oligonucleotide
tcctggcgggtaagt

SEQ ID NO: 1030 - DNA, 15 bp, Artificial Sequence
  Synthetic Oligonucleotide
tcctggcgggaaagt

SEQ ID NO: 1031 - DNA, 15 bp, Artificial Sequence
  Synthetic Oligonucleotide
tccgggcggggaagt

SEQ ID NO: 1032 - DNA, 15 bp, Artificial Sequence
  Synthetic Oligonucleotide
tcggggcggggaagt

SEQ ID NO: 1033 - DNA, 15 bp, Artificial Sequence
  Synthetic Oligonucleotide
tcccggcggggaagt

SEQ ID NO: 1034 - DNA, 15 bp, Artificial Sequence
  Synthetic Oligonucleotide
gggggacgttggggg

SEQ ID NO: 1035 - DNA, 20 bp, Artificial Sequence
  Synthetic Oligonucleotide
ggggttttttttttgggggg

SEQ ID NO: 1036 - DNA, 20 bp, Artificial Sequence
  Synthetic Oligonucleotide
ggggccccccccccgggggg

SEQ ID NO: 1037 - DNA, 21 bp, Artificial Sequence
  Synthetic Oligonucleotide
ggggttgttgttgttgggggg

SEQ ID NO: 1038 - DNA, 20 bp, Artificial Sequence
  Synthetic Oligonucleotide
ggggtcatcgatgagggggg

SEQ ID NO: 1039 - DNA, 20 bp, Artificial Sequence
  Synthetic Oligonucleotide
gggggacgatcgtcgggggg

SEQ ID NO: 1040 - DNA, 20 bp, Artificial Sequence
  Synthetic Oligonucleotide
ggggtcgacgtcgagggggg